SEQUENCE LISTING (55 sequences)

SEQ ID NO: 1 - DNA, 3768 bp, SARS coronavirus strain Tor2
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 2 - protein, 1255 aa, SARS coronavirus strain Tor2
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 3 - DNA, 3600 bp, Artificial Sequence
  S nucleotide fragment
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctc

SEQ ID NO: 4 - protein, 1200 aa, Artificial Sequence
  S peptide fragment
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWL

SEQ ID NO: 5 - DNA, 729 bp, Artificial Sequence
  S nucleotide fragment
agtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgtt

SEQ ID NO: 6 - protein, 243 aa, Artificial Sequence
  S peptide fragment
SGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFV

SEQ ID NO: 7 - DNA, 1740 bp, Artificial Sequence
  S nucleotide fragment
ggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcg

SEQ ID NO: 8 - protein, 580 aa, Artificial Sequence
  S peptide fragment
GYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICA

SEQ ID NO: 9 - DNA, 1266 bp, Artificial Sequence
  S nucleotide fragment
cagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 10 - protein, 421 aa, Artificial Sequence
  S peptide fragment
QKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 11 - DNA, 753 bp, Artificial Sequence
  S nucleotide fragment
taccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaa

SEQ ID NO: 12 - protein, 251 aa, Artificial Sequence
  S peptide fragment
YQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQ

SEQ ID NO: 13 - DNA, 603 bp, Artificial Sequence
  S nucleotide fragment
tccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggt

SEQ ID NO: 14 - protein, 201 aa, Artificial Sequence
  S peptide fragment
SNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGG

SEQ ID NO: 15 - DNA, 303 bp, Artificial Sequence
  S nucleotide fragment
tccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgat

SEQ ID NO: 16 - protein, 101 aa, Artificial Sequence
  S peptide fragment
SNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLND

SEQ ID NO: 17 - DNA, 300 bp, Artificial Sequence
  S nucleotide fragment
tatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggt

SEQ ID NO: 18 - protein, 100 aa, Artificial Sequence
  S peptide fragment
YGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGG

SEQ ID NO: 19 - DNA, 1983 bp, Artificial Sequence
  S nucleotide fragment
agtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaa

SEQ ID NO: 20 - protein, 661 aa, Artificial Sequence
  S peptide fragment
SGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQK

SEQ ID NO: 21 - DNA, 1569 bp, Artificial Sequence
  S nucleotide fragment
tctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggcct

SEQ ID NO: 22 - protein, 523 aa, Artificial Sequence
  S peptide fragment
SIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWP

SEQ ID NO: 23 - DNA, 864 bp, Artificial Sequence
  S nucleotide fragment
tctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaat

SEQ ID NO: 24 - protein, 288 aa, Artificial Sequence
  S peptide fragment
SIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLN

SEQ ID NO: 25 - DNA, 720 bp, Artificial Sequence
  S nucleotide fragment
gatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctc

SEQ ID NO: 26 - protein, 240 aa, Artificial Sequence
  S peptide fragment
DILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWL

SEQ ID NO: 27 - DNA, 1269 bp, SARS coronavirus strain Urbani
atgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataa

SEQ ID NO: 28 - protein, 422 aa, SARS coronavirus strain Urbani
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 29 - DNA, 633 bp, Artificial Sequence
  N nucleotide fragment
atgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatg

SEQ ID NO: 30 - protein, 211 aa, Artificial Sequence
  N peptide fragment
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARM

SEQ ID NO: 31 - DNA, 636 bp, Artificial Sequence
  N nucleotide fragment
gctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataa

SEQ ID NO: 32 - protein, 211 aa, Artificial Sequence
  N peptide fragment
ASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 33 - DNA, 603 bp, Artificial Sequence
  N nucleotide fragment
ggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaa

SEQ ID NO: 34 - protein, 201 aa, Artificial Sequence
  N peptide fragment
GKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYK

SEQ ID NO: 35 - DNA, 603 bp, Artificial Sequence
  N nucleotide fragment
actgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaa

SEQ ID NO: 36 - protein, 201 aa, Artificial Sequence
  N peptide fragment
TASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKK

SEQ ID NO: 37 - DNA, 753 bp, Artificial Sequence
  N nucleotide fragment
cgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgac

SEQ ID NO: 38 - protein, 251 aa, Artificial Sequence
  N peptide fragment
RNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAAD

SEQ ID NO: 39 - protein, 221 aa, SARS coronavirus GD322
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 40 - protein, 8 aa, Artificial Sequence
  Epitope tag
DYKDDDDK

SEQ ID NO: 41 - protein, 8 aa, Artificial Sequence
  Epitope tag
DLYDDDDK

SEQ ID NO: 42 - DNA, 3768 bp, SARS coronavirus Tor2; identical to SEQ ID NO: 1

SEQ ID NO: 43 - protein, 1255 aa, SARS coronavirus Tor2; identical to SEQ ID NO: 2

SEQ ID NO: 44 - DNA, 3768 bp, SARS coronavirus Frankfurt 1
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgattttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 45 - protein, 1255 aa, SARS coronavirus Frankfurt 1
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDFGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 46 - DNA, 3768 bp, SARS coronavirus TW5
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 47 - protein, 1255 aa, SARS coronavirus TW5
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 48 - DNA, 3768 bp, SARS coronavirus GD03T0013
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttgacgaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgttgtttctaaacccatgggtacacggacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacacttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagaggaaaagaatttctaattgtgttgctgattactctgtgctctacaactcaacatctttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctctcctgatggcaaaccttgcaccccacctgctcctaattgttattggccattaaatggttatggtttttacaccactagtggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacattaattcatgcagaacaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttcttcattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcagacaactaaatcgtgcactctcaggtattgctgctgaacaggatcgcaacacacgtgaagtgttcgttcaagtcaaacaaatgtacaaaaccccaactttgaaagattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgcggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaagaagaaattgaccgcctcaatgaggtcgctaaaaatttaaatgaatcactcatcgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 49 - protein, 1255 aa, SARS coronavirus GD03T0013
MFIFLLFLTLTSGSDLDRCTTFDDVQAPNYTQHTSSMRGVYYPDEIFRSDTLYLTQDLFLPFYSNVTGFHTINHTFDDPVIPFKDGIYFAATEKSNVVRGWVFGSTMNNKSQSVIIINNSTNVVIRACNFELCDNPFFVVSKPMGTRTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDTWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKRISNCVADYSVLYNSTSFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPAPNCYWPLNGYGFYTTSGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTLIHAEQLTPAWRIYSTGNNVFQTQAGCLIGAEHVDTSYECDIPIGAGICASYHTVSSLRSTSQKSIVAYTMSLGADSSIAYSNNTIAIPTNFSISITTEVMPVSMAKTSVDCNMYICGDSTECANLLLQYGSFCRQLNRALSGIAAEQDRNTREVFVQVKQMYKTPTLKDFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQEEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 50 - DNA, 3768 bp, SARS coronavirus BJ01
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttgacaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacacttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 51 - protein, 1255 aa, SARS coronavirus BJ01
MFIFLLFLTLTSGSDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDTWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 52 - DNA, 1269 bp, SARS coronavirus Urbani; identical to SEQ ID NO: 27

SEQ ID NO: 53 - protein, 422 aa, SARS coronavirus Urbani; identical to SEQ ID NO: 28

SEQ ID NO: 54 - DNA, 1269 bp, SARS coronavirus HB
atgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagctcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccggcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataa

SEQ ID NO: 55 - protein, 422 aa, SARS coronavirus HB
MSDNGPQSNQRSAPRITFGGPTDSTDNNQNGGRNGARPKQRRPQGLPNNTASWFTALTQHGKEELRFPRGQGVPINTNSGPDDQIGYYRRATRRARGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNRLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

(19) United States
(12) Patent Application Publication
(10) Pub. No.: US 2006/0286124 A1
(43) Pub. Date: Dec. 21, 2006

(54) Vaccine compositions and methods of treating coronavirus infection

(75) Inventors: David S. Burt, Dollard des Ormeaux (CA); Mark A. Reddish, Woodinville, WA (US); Mary ChaoHong Hu, Bothell, WA (US); George H. Lowell, Hampstead (CA); David Hugh Jones, Baie d'Urfe (CA)

Correspondence Address: SEED INTELLECTUAL PROPERTY LAW GROUP PLLC, 701 FIFTH AVE, SUITE 5400, SEATTLE, WA, 98104, US

(73) Assignee: ID Biomedical Corporation of Quebec, Laval (CA)

(21) Appl. No.: 11/173,793

(22) Filed: Jun. 30, 2005

Related U.S. Application Data
(60) Provisional application No. 60/584,704, filed on Jun. 30, 2004.

(51) Int. Cl. A61K 39/215; C12Q 1/70; C07H 21/04; C12P 21/06; C12N 15/86; C07K 14/165

(52) U.S. Cl. 424/221.1; 435/5; 435/69.1; 435/325; 530/350; 536/23.72; 435/456

(57) ABSTRACT

The present disclosure relates to compositions and methods for treating or preventing coronavirus infections. For example, compositions are provided that comprise a coronavirus S protein or N protein, fragment, or variant thereof, capable of eliciting a protective humoral and/or cell-mediated immune response, which compositions are useful for treating or preventing infection by coronavirus, such as the causative agent of SARS. Also, coronavirus S protein and N protein immunogen compositions are provided that include an adjuvant, such as Proteosome or Protollin, which may be used for treating or preventing infection caused by a coronavirus, such as a SARS coronavirus.

### CROSS-REFERENCE TO RELATED APPLICATION

This application claims the benefit of U.S. Provisional Patent Application No. 60/584,704 filed Jun. 30, 2004, which is herein incorporated by reference in its entirety.

### STATEMENT OF GOVERNMENT INTEREST

This invention was made in part with research funds from the National Institutes of Health under Grant No. UC1 AI062600-01. The government may have certain rights in this invention.

### STATEMENT REGARDING SEQUENCE LISTING SUBMITTED ON CD-ROM

The Sequence Listing associated with this application is provided on CD-ROM in lieu of a paper copy, and is hereby incorporated by reference into the application. Three CD-ROMs are provided, each containing identical copies of the sequence listing: CD-ROM No. 1 is labeled COPY 1 and contains the file 404.app.txt which is 177 KB and created on Jun. 30, 2005; CD-ROM No. 2 is labeled COPY 2 and contains the file 404.app.txt which is 177 KB and created on Jun. 30, 2005; CD-ROM No. 3 is labeled CRF (Computer Readable Form) and contains the file 404.app.txt which is 177 KB and created on Jun. 30, 2005.

### FIELD OF THE INVENTION

The present disclosure relates generally to vaccine compositions of coronavirus antigens and, more specifically, to compositions comprising one or more coronavirus immunogens (including S protein, N protein, M protein, and the like) and variants thereof, and uses of such compositions for eliciting a protective immune response to treat or prevent a coronavirus infection.

### DESCRIPTION OF THE RELATED ART

Since 1979, thirty new human viral diseases have emerged and, notably, most have been transmitted from animals to humans. One of the latest examples is the recent outbreak of Severe Acute Respiratory Syndrome (SARS). SARS is an emergent disease that appeared suddenly in November 2002 in the Guangdong Province of the People's Republic of China. In a short amount of time, the disease spread to other Asian countries and then spread in rapid succession to North America and Europe (WHO. Severe Acute Respiratory Syndrome (SARS). Wkly. Epidemiol. Rec. 78: 81, 2003). Within nine months of the initial appearance of SARS, nearly 8,500 cases were reported, with a mortality rate of about 10%. Clinically, the disease is characterized by fever, dyspnoea, lymphopenia, and pulmonary lesions, indicating diffuse alveolar damage (Nicholls et al., Lancet 361: 1773, 2003). Several candidate agents were suggested as the causal agent of the disease, but the search narrowed down to a previously unknown coronavirus (a group 2 Coronavirus; SARS-CoV or SCV), which, alone or in combination with human metapneumovirus, is now accepted as the primary cause of SARS (Ksiazek et al., N. Engl. J. Med. 348: 1953, 2003; Drosten et al., N. Engl. J. Med. 348: 1967, 2003; Kuiken et al., Lancet 362: 263, 2003; Fouchier et al., Nature 423: 240, 2003).

Coronaviruses are plus-strand RNA viruses that cause disease in animals and humans. A coronavirus infection can be systemic or localized. When localized, the coronavirus will infect only a few cell types, such as epithelial cells of the respiratory or enteric tract, and macrophages.

With such a new disease, many unresolved issues remain, even including the mode of transmission of the causal agent of SARS. Some clues can be gleaned from the 2003 epidemic: (a) nosocomial spread accounted for some 20-60% of the reported cases in various locations worldwide; (b) health care workers accounted for about 50% of cases in Toronto, Canada; and (c) spread was also common within households. The implication of this epidemiological data is profound—for example, if hospital closures were to be deemed necessary, most hospitals in the U.S. would face financial ruin (it has been estimated that a closure of just 2 weeks would leave most hospitals facing bankruptcy), while the high rates of infection within the health care worker population would stretch resources to the limit and would take a heavy toll on the workforce. During the 2003 epidemic entire workforces were sent home, and factories, companies, offices, and other businesses were temporarily closed. Furthermore, areas of perceived SARS hotspots were shunned, conferences were cancelled, and tourism industries suffered. Thus, a future epidemic could have far-reaching economic repercussions.

The major risk for transmission of the SARS virus is apparently by droplet exposure and close personal contact; therefore, strategies to reduce transmission of the SARS virus should parallel or mimic those used to limit other respiratory tract infections, i.e., reduce immediate contact and use barrier precautions against exposure to droplets. However, because the incubation period lasts from 2-10 days and non-specific initial symptoms are similar to those of other respiratory tract infections, such as influenza, the greatest risk for spread of SARS is undetected cases.

Thus, a need exists for alternative prophylactic strategies or therapies to treat or prevent coronavirus infections, such as those found in humans (e.g., infections resulting in SARS). For example, a need exists for identifying and developing vaccine compositions against coronavirus infections that can elicit a protective immune response. Furthermore, vaccine formulations are needed that can be delivered directly to or in close proximity to the site of infection to maximize therapeutic effectiveness. The present invention meets such needs and further provides other related advantages.

### BRIEF SUMMARY OF THE INVENTION

Briefly, the present invention relates to compositions and methods useful for treating or preventing a coronavirus infection, such as a SARS coronavirus infection. The compositions comprise, for example, a coronavirus S protein immunogen described herein and an adjuvant such as a Proteosome or Protollin™, that are capable of eliciting a protective immune response in a subject or host. In one embodiment, the invention provides a method for treating or preventing a coronavirus infection, comprising administering to a subject in need thereof a composition comprising (a) an adjuvant; (b) a pharmaceutically acceptable excipient; and (c) at least one coronavirus S protein immunogen comprising an amino acid sequence set forth in SEQ ID NO:4, SEQ ID NO:6, SEQ ID NO:8, SEQ ID NO:10, SEQ ID NO:12, SEQ ID NO:14, SEQ ID NO:16, or SEQ ID NO:18, wherein said at least one S protein immunogen is capable of eliciting a protective immune response against coronavirus. In certain embodiments, the at least one coronavirus S protein immunogen is at least 90% identical or at least 80% identical to an amino acid sequence set forth in SEQ ID NO:4, SEQ ID NO:6, SEQ ID NO:8, SEQ ID NO:10, SEQ ID NO:12, SEQ ID NO:14, SEQ ID NO:16, or SEQ ID NO:18. In a particular embodiment, the at least one coronavirus S protein immunogen further comprises a hydrophobic moiety, and in certain particular embodiments, the hydrophobic moiety is a hydrophobic polypeptide or a lipid. In another embodiment, the excipient is a liposome. In other particular embodiments, the adjuvant is alum, Freund's adjuvant, a Proteosome, or Protollin. In one embodiment, at least two S protein immunogens are administered. In another embodiment, the at least one coronavirus S protein immunogen is linked to a second amino acid sequence, and in certain embodiments the at least one coronavirus S protein immunogen is fused to the second amino acid sequence to form a fusion protein. In one embodiment, the second amino acid sequence is a tag or an enzyme. In a certain embodiment, the tag is a histidine tag. In certain embodiments, the coronavirus infection is caused by at least one or at least two coronaviruses selected from a group 1 coronavirus, a group 2 coronavirus, a group 3 coronavirus, and a SARS group coronavirus. In a particular embodiment, the coronavirus infection is caused by a human coronavirus, wherein the human coronavirus is SARS-CoV. In other embodiments, the composition is administered by a route selected from enteral, parenteral, transdermal, transmucosal, nasal, and inhalation, and in a particular embodiment, the composition is administered nasally. In one embodiment of the invention, the immune response elicited comprises at least one antibody that specifically binds to the at least one coronavirus S protein immunogen.

The invention also provides a composition comprising (a) at least one coronavirus S protein immunogen that comprises an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26; and (b) a Proteosome or Protollin, wherein said S protein immunogen is capable of eliciting a protective immune response. In a certain embodiment, the at least one coronavirus S protein immunogen comprises an amino acid sequence set forth in SEQ ID NO: 2 or SEQ ID NO:4. In certain embodiments, the at least one coronavirus S protein immunogen is at least 90% identical or at least 80% identical to an amino acid sequence set forth in SEQ ID NO:4, SEQ ID NO:6, SEQ ID NO:8, SEQ ID NO:10, SEQ ID NO:12, SEQ ID NO:14, SEQ ID NO:16, or SEQ ID NO:18. In a particular embodiment, the at least one coronavirus S protein immunogen further comprises a hydrophobic moiety, and in certain particular embodiments, the hydrophobic moiety is a hydrophobic polypeptide or a lipid. In one embodiment, at least two S protein immunogens are administered. In another embodiment, the at least one coronavirus S protein immunogen is linked to a second amino acid sequence, and in certain embodiments the at least one coronavirus S protein immunogen is fused to the second amino acid sequence to form a fusion protein. In one embodiment, the second amino acid sequence is a tag or an enzyme. In a certain embodiment, the tag is a histidine tag. In one embodiment, the composition further comprises a pharmaceutically acceptable excipient. In another embodiment, the at least one S protein immunogen is fused in frame to at least one second S protein immunogen comprising an amino acid sequence selected from an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26 to form a fusion protein.

In another embodiment, a composition is provided that comprises (a) a Proteosome or Protollin; and (b) a multivalent fusion coronavirus immunogen polypeptide. In still another embodiment, a method is provided for treating or preventing a coronavirus infection, comprising administering to a subject in need thereof the composition comprising a Proteosome or Protollin and a multivalent fusion coronavirus immunogen polypeptide.

Also provided herein are methods for treating or preventing a coronavirus infection that comprise a composition comprising (a) at least one coronavirus S protein immunogen that comprises an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26; and (b) a Proteosome or Protollin, wherein said S protein immunogen is capable of eliciting a protective immune response. In a certain embodiment, the at least one coronavirus S protein immunogen comprises an amino acid sequence set forth in SEQ ID NO: 2 or SEQ ID NO:4. In certain embodiments, the at least one coronavirus S protein immunogen is at least 90% identical or at least 80% identical to an amino acid sequence set forth in SEQ ID NO:4, SEQ ID NO:6, SEQ ID NO:8, SEQ ID NO:10, SEQ ID NO:12, SEQ ID NO:14, SEQ ID NO:16, or SEQ ID NO:18. In a particular embodiment, the at least one coronavirus S protein immunogen further comprises a hydrophobic moiety, and in certain particular embodiments, the hydrophobic moiety is a hydrophobic polypeptide or a lipid. In one embodiment, at least two S protein immunogens are administered. In another embodiment, the at least one coronavirus S protein immunogen is linked to a second amino acid sequence, and in certain embodiments the at least one coronavirus S protein immunogen is fused to the second amino acid sequence to form a fusion protein. In one embodiment, the second amino acid sequence is a tag or an enzyme. In a certain embodiment, the tag is a histidine tag. In one embodiment, the composition further comprises a pharmaceutically acceptable excipient. In another embodiment, the at least one S protein immunogen is fused in frame to at least one second S protein immunogen comprising an amino acid sequence selected from an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26 to form a fusion protein. In a particular embodiment, a method is provided for treating or preventing a coronavirus infection wherein the composition comprises Protollin and at least one coronavirus S protein immunogen, wherein the at least one coronavirus S protein immunogen comprises the amino acid sequence set forth in either SEQ ID NO:2 or SEQ ID NO:4.

In another embodiment, the invention provides a composition comprising (a) at least one coronavirus S protein immunogen that comprises an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26; and (b) a Proteosome or Protollin, wherein the S protein immunogen is capable of eliciting a protective immune response. In a particular embodiment, the at least one coronavirus S protein immunogen comprises an amino acid sequence at least 90% identical to the amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26, which S protein immunogen is capable of eliciting a protective immune response. In certain other embodiments, the at least one coronavirus S protein immunogen comprises an amino acid sequence at least 80% identical to the amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26, which S protein immunogen is capable of eliciting a protective immune response (that is, has at least one epitope that elicits or is capable of eliciting a protective immune response). In particular embodiments, the coronavirus S protein immunogen comprises an amino acid sequence that is identical to, that is 90% identical to, or that is 80% identical to the amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, or SEQ ID NO:18, wherein the S protein immunogen is capable of eliciting a protective immune response. In other particular embodiments, the at least one coronavirus S protein immunogen comprises an amino acid sequence set forth in SEQ ID NO: 2 or SEQ ID NO:4, an amino acid sequence that is at least 90% identical to SEQ ID NO:2 or 4, or an amino acid sequence that is at least 80% identical to SEQ ID NO:2 or 4, wherein the S protein immunogen is capable of eliciting a protective immune response. In particular embodiments, the composition comprises (1) a Proteosome or Protollin and (2) at least one coronavirus S protein immunogen, wherein the S protein immunogen comprises an amino acid sequence set forth in SEQ ID NO: 2 or SEQ ID NO:4. In a particular embodiment, the S protein immunogen further comprises a hydrophobic moiety, and in other particular embodiments, the hydrophobic moiety is a hydrophobic polypeptide or a lipid. In other embodiments, the at least one S protein immunogen is linked to a second amino acid sequence, and in a particular embodiment, the at least one coronavirus S protein immunogen is fused to the second amino acid sequence to form a fusion protein. In certain embodiments, the second amino acid sequence is a tag or an enzyme, and in a specific embodiment, the second amino acid sequence is a histidine tag. The present invention also provides the aforementioned compositions that further comprise at least one coronavirus N protein immunogen, wherein the N protein immunogen comprises an amino acid sequence selected from SEQ ID NO:28, SEQ ID NO:30, SEQ ID NO:32, SEQ ID NO:34, SEQ ID NO:36, and SEQ ID NO:38. In another embodiment, the aforementioned compositions further comprise at least one M protein immunogen, wherein the at least one M protein immunogen is capable of eliciting an immune response. In a specific embodiment, the M protein immunogen comprises the sequence set forth in GenBank Accession No. AAU07933 (SEQ ID NO:39).

In other embodiments, a composition comprising a Proteosome or Protollin and at least one S protein immunogen (as described herein) is provided wherein the at least one S protein immunogen is fused in frame to at least one second S protein immunogen comprising an amino acid sequence selected from the amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26 to form a fusion protein. In still another embodiment, the at least one S protein immunogen is fused in frame to a coronavirus N protein immunogen comprising an amino acid sequence selected from the amino acid sequence set forth in SEQ ID NO:28; SEQ ID NO:30, SEQ ID NO:32, SEQ ID NO:34, SEQ ID NO:36, and SEQ ID NO:38.

The invention also provides a composition comprising (a) at least one N protein immunogen that comprises an amino acid sequence set forth in SEQ ID NO:28, SEQ ID NO:30, SEQ ID NO:32, SEQ ID NO:34, SEQ ID NO:36, or SEQ ID NO:38; and (b) a Proteosome or Protollin, wherein the N protein immunogen is capable of eliciting a protective immune response. In certain embodiments, the N protein immunogen comprises an amino acid sequence that is at least 90% identical to an amino acid sequence selected from the amino acid sequence set forth in SEQ ID NO:28, SEQ ID NO:30, SEQ ID NO:32, SEQ ID NO:34, SEQ ID NO:36, and SEQ ID NO:38, and in certain other embodiments, the N protein immunogen comprises an amino acid sequence that is at least 80% identical to an amino acid sequence selected from the amino acid sequence set forth in SEQ ID NO:28, SEQ ID NO:30, SEQ ID NO:32, SEQ ID NO:34, SEQ ID NO:36, and SEQ ID NO:38. In certain embodiments, the N protein immunogen further comprises a hydrophobic moiety, and in other certain embodiments, the hydrophobic moiety is a hydrophobic polypeptide or a lipid.

In another embodiment, the invention provides a composition comprising (a) a Proteosome or Protollin; and (b) a multivalent fusion coronavirus immunogen polypeptide. In certain embodiments, the multivalent fusion coronavirus immunogen comprises at least two S protein immunogens or fragments thereof. In certain embodiments, the multivalent fusion coronavirus immunogen comprises at least two S protein immunogens that are selected from an S protein immunogen comprising an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26. In certain other embodiments, the multivalent fusion coronavirus immunogen comprises at least one S protein immunogen comprising an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:26 and at least one coronavirus N protein immunogen comprising an amino acid sequence set forth in SEQ ID NO:28, SEQ ID NO:30, SEQ ID NO:32, SEQ ID NO:34, SEQ ID NO:36, or SEQ ID NO:38.

In specific embodiments, any one of the compositions described herein (including those described above) further comprises a pharmaceutically acceptable excipient. In other specific embodiments, the invention provides any one of the compositions described herein (including those described above) for use in treating or preventing a coronavirus infection. Also provided herein, is the use of any one of the compositions described herein (including those described above) for the manufacture of a medicament for treating or preventing a coronavirus infection. In particular embodiments, the coronavirus infection is caused by at least one of a group 1 coronavirus, group 2 coronavirus, a group 3 coronavirus, and a SARS group coronavirus, and in other embodiments, the coronavirus infection is caused by at least two of a group 1, group 2, group 3, and SARS group coronavirus. In a particular embodiment, the coronavirus infection is caused by a human coronavirus, and in another particular embodiment the human coronavirus is SARS-CoV.

In one embodiment, the present invention provides a method for treating or preventing a coronavirus infection, comprising administering to a subject in need thereof any one of the compositions described herein. In a particular embodiment, a method for treating or preventing a coronavirus infection, comprises administering to a subject in need thereof a composition comprising (a) a Proteosome or Protollin; (b) at least one coronavirus S protein immunogen that comprises an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26; and (c) at least one N protein immunogen that comprises an amino acid sequence that is selected from the amino acid sequence set forth in SEQ ID NO:28, SEQ ID NO:30, SEQ ID NO:32, SEQ ID NO:34, SEQ ID NO:36, and SEQ ID NO:38. In a particular embodiment, the at least one coronavirus N protein immunogen is at least 90% identical to the amino acid sequence set forth in SEQ ID NO:28, SEQ ID NO:30, SEQ ID NO:32, SEQ ID NO:34, SEQ ID NO:36, or SEQ ID NO:38, and in another particular embodiment, the at least one coronavirus N protein immunogen is at least 80% identical to the amino acid sequence set forth in SEQ ID NO:28, SEQ ID NO:30, SEQ ID NO:32, SEQ ID NO:34, SEQ ID NO:36, or SEQ ID NO:38. In another embodiment, the invention provides a method for treating or preventing a coronavirus infection, comprising administering to a subject in need thereof a composition comprising: (a) a Proteosome or Protollin; (b) at least one coronavirus S protein immunogen that comprises an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26. In a particular embodiment, the S protein immunogen comprises an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, or SEQ ID NO:18. In certain embodiments, the methods comprise at least one coronavirus S protein immunogen wherein the S protein immunogen is at least 90% identical to the amino acid sequence set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26. In another certain embodiment, the at least one coronavirus S protein immunogen is at least 80% identical to the amino acid sequence set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26. In another particular embodiment, the S protein immunogen comprises an amino acid sequence set forth in SEQ ID NO: 2 or SEQ ID NO:4, or comprises an amino acid sequence at least 90% identical to the amino acid sequence set forth in SEQ ID NO:2 or SEQ ID NO:4, or comprises an amino acid sequence at least 80% identical to the amino acid sequence set forth in SEQ ID NO:2 or SEQ ID NO:4, wherein the S protein immunogen is capable of elicting a protective immune response. In certain embodiments, the at least one coronavirus S protein immunogen further comprises a hydrophobic moiety, wherein the hydrophobic moiety is a hydrophobic polypeptide or a lipid. In other embodiments, wherein the method comprises administering at least one S protein immunogen and at least one N protein immunogen, the at least one coronavirus N protein immunogen further comprises a hydrophobic moiety, or the at least one coronavirus S protein immunogen further comprises a hydrophobic moiety, or the at least one N protein immunogen and the at least one S protein immunogen comprises a hydrophobic moiety, wherein the hydrophobic moiety is a hydrophobic polypeptide or a lipid. In other embodiments of these methods, the composition further comprises at least one M protein immunogen, wherein the M protein immunogen is capable of eliciting an immune response. In a specific embodiment, the M protein immunogen comprises the sequence set forth in GenBank Accession No. AAU07933 (SEQ ID NO:39). In certain other embodiments, the at least one coronavirus S protein immunogen is linked to a second amino acid sequence, and in specific embodiments, the S protein immunogen is fused to the second amino acid sequence to form a fusion protein. In a specific embodiment, the second amino acid sequence is a tag or an enzyme, and in other specific embodiments, the tag is a histidine tag. In other particular embodiments, wherein the method comprises the at least one N protein immunogen, the at least one coronavirus N protein immunogen is linked to a second amino acid sequence, and in specific embodiments, the N protein immunogen is fused to the second amino acid sequence to form a fusion protein. In a specific embodiment, the second amino acid sequence is a tag or an enzyme, and in other specific embodiments, the tag is a histidine tag. In another embodiment, the coronavirus infection is caused by at least one of a group 1 coronavirus, group 2 coronavirus, a group 3 coronavirus, and a SARS group coronavirus, and in other embodiments, the coronavirus infection is caused by at least two of a group 1, group 2, group 3, and SARS group coronavirus. In a particular embodiment, the coronavirus infection is caused by a human coronavirus, and in another particular embodiment the human coronavirus is SARS-CoV. In certain embodiments of the methods, the composition is administered by a route selected from enteral, parenteral, transdermal, transmucosal, nasal, and inhalation. In a particular embodiment, the composition is administered nasally. In particular embodiments, the at least one coronavirus S protein immunogen comprises the amino acid sequence set forth in SEQ ID NO:2 or SEQ ID NO:4. In another particular embodiment, the invention provides a method for treating or preventing a coronavirus infection, comprising administering to a subject in need thereof a composition that comprises Protollin and at least one coronavirus S protein immunogen, wherein the at least one coronavirus S protein immunogen comprises the amino acid sequence set forth in either SEQ ID NO:2 or SEQ ID NO:4.

Also provided by the present invention is a method for treating or preventing a coronavirus infection, comprising administering to a subject in need thereof a composition comprising a pharmaceutically acceptable excipient and at least one coronavirus S protein immunogen comprising an amino acid sequence set forth in SEQ ID NO:4, SEQ ID NO:6, SEQ ID NO:8, SEQ ID NO:10, SEQ ID NO:12, SEQ ID NO:14, SEQ ID NO:16, or SEQ ID NO:18, wherein said at least one S protein immunogen is capable of eliciting a protective immune response against coronavirus. In certain embodiments, the at least one coronavirus S protein immunogen is at least 90% identical to the amino acid sequence set forth in SEQ ID NO:4, SEQ ID NO:6, SEQ ID NO:8, SEQ ID NO:10, SEQ ID NO:12, SEQ ID NO:14, SEQ ID NO:16, or SEQ ID NO:18, and in other certain embodiments, the at least one coronavirus S protein immunogen is at least 80% identical to the amino acid sequence set forth in SEQ ID NO:4, SEQ ID NO:6, SEQ ID NO:8, SEQ ID NO:10, SEQ ID NO:12, SEQ ID NO:14, SEQ ID NO:16, or SEQ ID NO:18. In a particular embodiment, the coronavirus S protein immunogen further comprises a hydrophobic moiety, and in other particular embodiments, the hydrophobic moiety is a hydrophobic polypeptide or a lipid. In another embodiment, the composition further comprises an adjuvant, and in a particular embodiment, the adjuvant is alum, Freund's adjuvant, a Proteosome, or Protollin. In another embodiment, the composition further comprises at least one M protein immunogen, wherein the M protein immunogen is capable of eliciting an immune response, and in a particular embodiment, the M protein immunogen comprises the amino acid sequence set forth in GenBank Accession No. AAU07933. In certain embodiments, the method comprises administering at least two S protein immunogens. In another embodiment, the at least one coronavirus S protein immunogen is linked to a second amino acid sequence, and in another particular embodiment, the at least one coronavirus S protein immunogen is fused to the second amino acid sequence to form a fusion protein. In a certain embodiment, the second amino acid sequence is a tag or an enzyme, and in a certain particular embodiment, the tag is a histidine tag. In particular embodiments, the coronavirus infection is caused by a group 1 coronavirus, a group 2 coronavirus, a group 3 coronavirus, or a a SARS group coronavirus. In other embodiments, the coronavirus infection is caused by at least two of a group 1, group 2, group 3, and SARS group coronavirus. In a specific embodiment, the coronavirus infection is caused by a human coronavirus, and in another specific embodiment, the human coronavirus is SARS-CoV. In still another embodiment, the composition is administered by a route selected from enteral, parenteral, transdermal, transmucosal, nasal, and inhalation. In a particular embodiment, the composition is administered nasally. In particular embodiments, the immune response comprises eliciting at least one antibody that specifically binds to the at least one coronavirus S protein immunogen.

In still another embodiment, the invention provides a method for treating or preventing a coronavirus infection, comprising administering to a subject in need thereof a composition comprising (a) a pharmaceutically acceptable excipient; (b) at least one coronavirus S protein immunogen; and (c) at least one coronavirus N protein immunogen, wherein the at least one S protein immunogen is selected from an amino acid sequence set forth in SEQ ID NO:4 SEQ ID NO:4, SEQ ID NO:6, SEQ ID NO:8, SEQ ID NO:10, SEQ ID NO:12, SEQ ID NO:14, SEQ ID NO:16, and SEQ ID NO:18, and wherein the at least one N protein immunogen is selected from an amino acid sequence set forth in SEQ ID NO:30, SEQ ID NO:32, SEQ ID NO:34, SEQ ID NO:36, or SEQ ID NO:38, wherein said at least one coronavirus S protein immunogen and at least one coronavirus N immunogen are capable of eliciting a protective immune response against coronavirus. In certain embodiments, the at least one coronavirus S protein immunogen is at least 90% identical to the amino acid sequence set forth in SEQ ID NO:4, SEQ ID NO:6, SEQ ID NO:8, SEQ ID NO:10, SEQ ID NO:12, SEQ ID NO:14, SEQ ID NO:16, or SEQ ID NO:18, and in other certain embodiments, the at least one coronavirus S protein immunogen is at least 80% identical to the amino acid sequence set forth in SEQ ID NO:4, SEQ ID NO:6, SEQ ID NO:8, SEQ ID NO:10, SEQ ID NO:12, SEQ ID NO:14, SEQ ID NO:16, or SEQ ID NO:18. In certain embodiments, the at least one coronavirus N protein immunogen is at least 90% identical to the amino acid sequence set forth in SEQ ID NO:30, SEQ ID NO:32, SEQ ID NO:34, SEQ ID NO:36, or SEQ ID NO:38, and in certain other embodiments, the at least one coronavirus N protein immunogen is at least 80% identical to the amino acid sequence set forth in SEQ ID NO:30, SEQ ID NO:32, SEQ ID NO:34, SEQ ID NO:36, or SEQ ID NO:38. In a particular embodiment, the coronavirus S protein immunogen further comprises a hydrophobic moiety, and in other particular embodiments, the hydrophobic moiety is a hydrophobic polypeptide or a lipid. In a particular embodiment, the coronavirus N protein immunogen further comprises a hydrophobic moiety, and in other particular embodiments, the hydrophobic moiety is a hydrophobic polypeptide or a lipid. In another embodiment, the composition further comprises an adjuvant, and in a particular embodiment, the adjuvant is alum, Freund's adjuvant, a Proteosome, or Protollin. In another embodiment, the composition further comprises at least one M protein immunogen, wherein the M protein immunogen is capable of eliciting an immune response, and in a particular embodiment, the M protein immunogen comprises the amino acid sequence set forth in GenBank Accession No. AAU07933. In certain embodiments, the method comprises administering at least two S protein immunogens. In another embodiment, the at least one coronavirus S protein immunogen is linked to a second amino acid sequence, and in another particular embodiment, the at least one coronavirus S protein immunogen is fused to the second amino acid sequence to form a fusion protein. In a certain embodiment, the second amino acid sequence is a tag or an enzyme, and in a certain particular embodiment, the tag is a histidine tag. In certain embodiments, the method comprises administering at least two N protein immunogens. In another embodiment, the at least one coronavirus N protein immunogen is linked to a second amino acid sequence, and in another particular embodiment, the at least one coronavirus N protein immunogen is fused to the second amino acid sequence to form a fusion protein. In a certain embodiment, the second amino acid sequence is a tag or an enzyme, and in a certain particular embodiment, the tag is a histidine tag. In particular embodiments, the coronavirus infection is caused by a group 1 coronavirus, a group 2 coronavirus, a group 3 coronavirus, or a SARS group coronavirus. In other embodiments, the coronavirus infection is caused by at least two of a group 1, group 2, group 3, and SARS group coronavirus. In a specific embodiment, the coronavirus infection is caused by a human coronavirus, and in another specific embodiment, the human coronavirus is SARS-CoV. In still another embodiment, the composition is administered by a route selected from enteral, parenteral, transdermal, transmucosal, nasal, and inhalation. In a particular embodiment, the composition is administered nasally. In particular embodiments, the immune response comprises eliciting at least one antibody that specifically binds to the at least one coronavirus S protein immunogen, and in other particular embodiments, the immune response comprises eliciting at least one antibody that specifically binds to the at least one coronavirus N protein immunogen

In another embodiment, a method is provided for treating or preventing a coronavirus infection, comprising administering to a subject in need thereof a composition comprising a pharmaceutically acceptable excipient and at least one coronavirus N protein immunogen comprising an amino acid sequence set forth in SEQ ID NO:30, SEQ ID NO:32, SEQ ID NO:34, SEQ ID NO:36, or SEQ ID NO:38, wherein said at least one coronavirus N protein immunogen is capable of eliciting a protective immune response against coronavirus. In certain embodiments, the at least one coronavirus N protein immunogen is at least 90% identical to the amino acid sequence set forth in SEQ ID NO:30, SEQ ID NO:32, SEQ ID NO:34, SEQ ID NO:36, or SEQ ID NO:38, and in certain other embodiments, the at least one coronavirus N protein immunogen is at least 80% identical to the amino acid sequence set forth in SEQ ID NO:30, SEQ ID NO:32, SEQ ID NO:34, SEQ ID NO:36, or SEQ ID NO:38. In a particular embodiment, the coronavirus N protein immunogen further comprises a hydrophobic moiety, and in other particular embodiments, the hydrophobic moiety is a hydrophobic polypeptide or a lipid. In a particular embodiment, the excipient is a liposome. In another embodiment, the composition further comprises an adjuvant, and in a particular embodiment, the adjuvant is alum, Freund's adjuvant, a Proteosome, or Protollin. In another embodiment, the composition further comprises at least one M protein immunogen, wherein the M protein immunogen is capable of eliciting an immune response, and in a particular embodiment, the M protein immunogen comprises the amino acid sequence set forth in GenBank Accession No. AAU07933. In certain embodiments, the method comprises administering at least two N protein immunogens. In another embodiment, the at least one coronavirus N protein immunogen is linked to a second amino acid sequence, and in another particular embodiment, the at least one coronavirus N protein immunogen is fused to the second amino acid sequence to form a fusion protein. In a certain embodiment, the second amino acid sequence is a tag or an enzyme, and in a certain particular embodiment, the tag is a histidine tag. In particular embodiments, the coronavirus infection is caused by a group 1 coronavirus, a group 2 coronavirus, a group 3 coronavirus, or a a SARS group coronavirus. In other embodiments, the coronavirus infection is caused by at least two of a group 1, group 2, group 3, and SARS group coronavirus. In a specific embodiment, the coronavirus infection is caused by a human coronavirus, and in another specific embodiment, the human coronavirus is SARS-CoV. In still another embodiment, the composition is administered by a route selected from enteral, parenteral, transdermal, transmucosal, nasal, and inhalation. In a particular embodiment, the composition is administered nasally. In particular embodiments, the immune response comprises eliciting at least one antibody that specifically binds to the at least one coronavirus N protein immunogen.

The invention also provides a plurality of isolated antibodies produced by a method comprising administering to a subject a composition comprising a pharmaceutically acceptable excipient and at least one coronavirus S protein immunogen comprising an amino acid sequence set forth in SEQ ID NO:4, SEQ ID NO:6, SEQ ID NO:8, SEQ ID NO:10, SEQ ID NO:12, SEQ ID NO:14, SEQ ID NO:16, or SEQ ID NO:18, wherein said at least one S protein immunogen is capable of eliciting a protective immune response against coronavirus. In another embodiment, the invention provides a plurality of isolated antibodies produced by a method comprising administering to a subject a composition comprising (a) a pharmaceutically acceptable excipient; (b) at least one coronavirus S protein immunogen; and (c) at least one coronavirus N protein immunogen, wherein the at least one S protein immunogen is selected from an amino acid sequence set forth in SEQ ID NO:4 SEQ ID NO:4, SEQ ID NO:6, SEQ ID NO:8, SEQ ID NO:10, SEQ ID NO:12, SEQ ID NO:14, SEQ ID NO:16, and SEQ ID NO:18, and wherein the at least one N protein immunogen is selected from an amino acid sequence set forth in SEQ ID NO:30, SEQ ID NO:32, SEQ ID NO:34, SEQ ID NO:36, or SEQ ID NO:38, wherein said at least one coronavirus S protein immunogen and at least one coronavirus N immunogen are capable of eliciting a protective immune response against coronavirus. In still another embodiment, the invention provides a plurality of isolated antibodies produced by a method comprising administering to a subject a composition comprising a pharmaceutically acceptable excipient and at least one coronavirus N protein immunogen comprising an amino acid sequence set forth in SEQ ID NO:30, SEQ ID NO:32, SEQ ID NO:34, SEQ ID NO:36, or SEQ ID NO:38, wherein said at least one coronavirus N protein immunogen is capable of eliciting a protective immune response against coronavirus.

In another embodiment, is provided a composition comprising (a) at least one isolated antibody or antigen-binding fragment thereof that specifically-binds to at least one coronavirus S polypeptide comprising an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26; and (b) at least one isolated antibody or antigen-binding fragment thereof that specifically binds to at least one coronavirus N polypeptide that comprises an amino acid sequence set forth in SEQ ID NO:28, SEQ ID NO:30, SEQ ID NO:32, SEQ ID NO:34, SEQ ID NO:36, or SEQ ID NO:38, wherein the composition inhibits infection by a coronavirus. In particular embodiments, the composition further comprises a pharmaceutically acceptable excipient. In another embodiment, a method is provided for treating or preventing a coronavirus infection, comprising administering to a subject in need thereof a composition comprising (a) at least one isolated antibody or antigen-binding fragment thereof that specifically binds to at least one coronavirus S polypeptide comprising an amino acid sequence set forth in SEQ ID NO: 2, SEQ ID NO:4, SEQ ID NO:6, SEQ ID NO:8, SEQ ID NO:10, SEQ ID NO:12, SEQ ID NO: 14, SEQ ID NO: 16, SEQ ID NO:18, SEQ ID NO:20, SEQ ID NO:22, SEQ ID NO:24, or SEQ ID NO:26; and (b) at least one isolated antibody or antigen-binding fragment thereof that specifically binds to at least one coronavirus N polypeptide that comprises an amino acid sequence set forth in SEQ ID NO:28, SEQ ID NO:30, SEQ ID NO:32, SEQ ID NO:34, SEQ ID NO:36, or SEQ ID NO:38

These and other embodiments of the present invention will become evident upon reference to the following detailed description and attached drawings. In addition, all U.S. patents, U.S. patent application publications, U.S. patent applications, foreign patents, foreign patent applications, foreign patent application publications, and non-patent publications referred to in this application and/or listed in the Application Data Sheet, are incorporated herein by reference, in their entirety.

### BRIEF DESCRIPTION OF THE DRAWINGS

FIG. 1 shows a schematic of full-length S protein (S_{TM}) and an S protein variant (S_{TM-del}). The top of the schematic shows the nucleotide sequences that correspond to the front, middle, and back portions of the S protein. The hatched box represents the transmembrane (TM) domain.

FIG. 2 shows the serum (IgG) dose response in mice that were administered various amounts of S_{TM-del} protein alone (S_{TM-del} protein i.n.) or adjuvanted with Protollin™ (S_{TM-del} protein+Protollin™ i.n.).

FIG. 3A illustrates the titer of serum IgG in mice that were immunized intranasally (i.n.) with S_{TM-del} protein adjuvanted with Protollin™ or S_{TM-del} protein alone, and in mice that were immunized intramuscularly with S_{TM-del} protein adjuvanted with alum and in mice that received only PBS. FIG. 3B shows the titer of lung IgA from mice immunized intranasally (i.n.) with S_{TM-del} protein adjuvanted with Protollin or S_{TM-del} protein alone, and in mice that were immunized intramuscularly with S_{TM-del} protein adjuvanted with alum and in mice that received only PBS.

FIGS. 4A-4F presents the nucleotide sequence (SEQ ID NO:1) and amino acid sequence (SEQ ID NO:2) of S protein from SARS coronavirus strain Tor2.

FIGS. 5A-5B presents the nucleotide sequence (SEQ ID NO:27) and amino acid sequence (SEQ ID NO:28) of N protein from SARS coronavirus strain Urbani.

FIG. 6 illustrates serum IgG titers of mice (either anesthetized (with) or non-anesthetized (without)) that received intranasally 10 μg SARS S-protein (full-length) or S_{TM-del} protein (transmembrane deleted (ΔTM Deleted)) combined with various concentrations of Protollin™. Additional groups of mice received intramuscular injections of 10 μg SARS S-protein adsorbed to Alhydrogel® (F.L. i.m.) or 10 μg S_{TM-del} protein (Del i.m.).

FIG. 7 presents IgA titers in lung lavage and nasal washes from the mice immunized as described in the Brief Description of FIG. 6.

FIG. 8 illustrates release of cytokines from in vitro re-stimulated splenocytes from mice immunized with full-length S-protein and Protollin (S(FL)+Protollin™); full-length S-protein and Alhydrogel (S(FL)+Alum); and PBS alone.

### DETAILED DESCRIPTION OF THE INVENTION

As set forth above, described herein are compositions comprising at least one coronavirus S protein immunogen, at least one N protein immunogen, or at least one S protein immunogen and at least one N protein immunogen, fragments or variants thereof, which are capable of eliciting an immune response that is protective against an infection caused by a coronavirus. Also described herein are methods for making and using these S and N protein immunogens to treat or prevent coronavirus infections. Coronavirus immunogens (antigens) of the invention comprise at least one coronavirus virus-encoded polypeptide, such as a coronavirus virus S protein immunogen or N protein immunogen, or variants and fragments thereof, capable of eliciting an immune response, which includes a neutralizing antibody response and/or cell-mediated immunity. Coronavirus antigens (immunogens) may comprise one or more recombinantly or synthetically produced coronavirus polypeptides or may comprise one or more coronavirus polypeptides isolated from coronavirus viral particles or from coronavirus-infected host cells. Discussed in more detail below are coronavirus S and N protein immunogens, fragments, derivatives, and variants thereof, as well as representative compositions and therapeutic uses.

In certain embodiments, adjuvanted coronavirus virus S or N protein immunogens, fragments, derivatives, or variants thereof are provided. For example, the coronavirus virus S or N protein immunogens may be combined or admixed with Proteosomes or Protollin™. Proteosome (also referred to as Projuvant) combinations or mixtures are comprised of outer membrane proteins obtained from Gram-negative bacteria. Alternatively, Proteosomes can be combined with endogenous or exogenous liposaccharides (i.e., OMP:LPS, also referred to as Protollin). Therefore, these immunogenic compositions (vaccine compositions or formulations) are advantageous over other more typical adjuvanted vaccines in that proteosome technology-based adjuvants are capable of aiding in eliciting an innate immune response, an enhanced serological and mucosal response, and a specific immune response when “loaded” with one or more immunogens (or antigens) of interest (such as coronavirus virus S or N protein immunogens, and fragments, derivatives, or variants thereof, or other immunogens).

“Proteosome or Projuvant,” as used herein, refers to preparations of outer membrane proteins (OMPs, also known as porins) from Gram-negative bacteria, such as Neisseria species (see, e.g., Lowell et al., J. Exp. Med. 167:658, 1988; Lowell et al., Science 240:800, 1988; Lynch et al., Biophys. J. 45:104, 1984; Lowell, in “New Generation Vaccines” 2nd ed., Marcel Dekker, Inc., New York, Basil, Hong Kong, page 193, 1997; U.S. Pat. No. 5,726,292; U.S. Pat. No. 4,707,543), which are useful as a carrier or an adjuvant for immunogens, such as bacterial or viral antigens. Proteosomes are hydrophobic and safe for human use, and comparable in size to certain viruses. Proteosomes have the capability to auto-assemble into vesicle or vesicle-like OMP clusters of about 20 nm to about 800 nm, and to noncovalently incorporate, coordinate, associate (e.g., electrostatically or hydrophobically), or otherwise cooperate with protein antigens (Ags), particularly antigens that have a hydrophobic moiety. Any preparation method that results in the outer membrane protein component in vesicular or vesicle-like form, including multi-molecular membranous structures or molten globular-like OMP compositions of one or more OMPs, is included within the definition of Proteosome. Proteosomes may be prepared, for example, as described in the art (see, e.g., U.S. Pat. No. 5,726,292 or U.S. Pat. No. 5,985,284). Proteosomes prepared according to procedures set forth herein may also contain an endogenous lipopolysaccharide or lipooligosaccharide (LPS or LOS, respectively) originating from the bacteria used to produce the OMP porins (e.g., Neisseria species), which generally will be less than 2% of the total OMP preparation.

“Liposaccharide”, as used herein, refers to native (isolated or prepared synthetically with a native structure) or modified lipopolysaccharide or lipooligosaccharide (collectively, also referred to as “LPS”) derived from Gram-negative bacteria, such as Shigella flexneri or Plesiomonas shigelloides, or other Gram-negative bacteria (including Alcaligenes, Bacteroides, Bordetella, Borrellia, Brucella, Campylobacter, Chlamydia, Citrobacter, Edwardsiella, Ehrlicha, Enterobacter, Escherichia, Francisella, Fusobacterium, Gardnerella, Hemophilus, Helicobacter, Klebsiella, Legionella, Leptospira (including Leptospira interrogans), Moraxella, Morganella, Neiserria, Pasteurella, Proteus, Providencia, other Plesiomonas, Porphyromonas (including Porphyromonas gingivalis), Prevotella, Pseudomonas, Rickettsia, Salmonella, Serratia, other Shigella, Spirllum, Veillonella, Vibrio, or Yersinia species). The liposaccharide may be in a detoxified form (i.e., having the Lipid A core removed) or may be in a form that has not been detoxified. For example, an LPS that contains multiple lipid A species such as P. gingivalis LPS may be used in the compositions described herein (see, e.g., Darveau et al., Infect. Immun. 72:5041-51 (2004)). In the instant disclosure, liposaccharide need not be, and preferably is not, detoxified. The liposaccharide may be prepared, for example, as described in U.S. Patent Application Publication No. 2003/0044425.

“Proteosome: LPS or Protollin or IVX or IVX-908” as used herein refers to preparations of projuvant admixed as described herein (e.g., by the exogenous addition) with at least one kind of liposaccharide to provide an OMP-LPS composition (which can function as an immunostimulatory composition). Thus, the OMP-LPS adjuvant can be comprised of two of the basic components of Protollin, which include (1) an outer membrane protein preparation of Proteosomes (i.e., Projuvant) prepared from Gram-negative bacteria, such as Neisseria meningitidis, and (2) a preparation of one or more liposaccharides. A liposaccharide may be endogenous (i.e., naturally contained in the OMP Proteosome preparation), may be admixed or combined with an OMP preparation from an exogenously prepared liposaccharide (i.e., prepared from a different culture or microorganism than the OMP preparation), or may be a combination thereof. Such exogenously added LPS may be from the same Gram-negative bacterium from which the OMP preparation was made or from a different Gram-negative bacterium. Protollin should also be understood to optionally include lipids, glycolipids, glycoproteins, small molecules, or the like, and combinations thereof. The Protollin may be prepared, for example, as described in U.S. Patent Application Publication No. 2003/0044425.

Projuvant is generally used in conjunction with antigens (naturally-occurring or modified) that possess a naturally occurring, modified, or supplementary hydrophobic moiety (also referred to as a “foot” or “anchor”). Protollin (containing exogenously added LPS) can be used with an antigen that does not contain a hydrophobic foot domain and that can be largely hydrophilic in nature. Protollin can be admixed or combined with an antigen containing a hydrophobic foot, an antigen lacking a hydrophobic foot, or with a combination of antigens having and not having a hydrophobic portion or foot.

An immunogenic composition as used herein refers to any one or more compounds or agents or immunogens capable of priming, potentiating, activating, eliciting, stimulating, augmenting, boosting, amplifying, or enhancing an adaptive (specific) immune response, which may be cellular (T cell) or humoral (B cell), or a combination thereof. Preferably, the adaptive immune response is protective, which may include neutralization of a virus (decreasing or eliminating virus infectivity). A representative example of an immunogen is a microbial antigen (such as one or more coronavirus antigens).

In the present description, any concentration range, percentage range, ratio range, or integer range is understood to include the value of any integer within the recited range and, when appropriate, fractions thereof (such as one tenth and one hundredth of an integer, etc.), unless otherwise indicated. As used herein, “about” or “comprising essentially of” means±15%. As used herein, the use of an indefinite article, such as “a” or “an,” should be understood to refer to the singular and the plural of a noun or noun phrase (i.e., meaning “one or more” or “at least one” of the enumerated elements or components). The use of the alternative (e.g., “or”) should be understood to mean either one, both or any combination thereof of the alternatives. In addition, it should be understood that the individual compounds, or groups of compounds, derived from the various combinations of the sequences, structures, and substituents described herein, are disclosed by the present application to the same extent as if each compound or group of compounds was set forth individually. Thus, selection of particular sequences, structures, or substituents is within the scope of the present invention.

Coronavirus Immunogens

Compositions as described herein useful for treating and/or preventing a coronavirus infection comprises immunogenic coronavirus polypeptides, such as S protein, fragments, and variants thereof, and also includes a fusion of a coronavirus immunogen to other peptides or polypeptides (e.g., a hydrophobic amino acid sequence or a histidine tag or a non-S protein coronavirus polypeptide or fragment thereof) or other modifications (e.g. glycosylation). In certain embodiments, the immunogenic S polypeptides may comprise any portion of an S protein that has an epitope capable of eliciting a protective immune response (e.g., eliciting production of a neutralizing antibody and/or stimulating a cell-mediated immune response) against a coronavirus infection. Immunogenic polypeptides as described herein may be arranged, combined, or fused in a linear form, and each immunogen may or may not be reiterated, wherein the reiteration may occur once or multiple times, and may be located at the N-terminus, C-terminus, or internal to a linear sequence of immunogenic S or other coronavirus polypeptide immunogens. In addition, a plurality of different coronavirus immunogenic polypeptides (e.g., other S proteins, N proteins, M proteins, or other coronavirus polypeptides, and variants or fragments thereof) can be selected and mixed or combined into a cocktail composition to provide a multivalent vaccine for use in eliciting a protective immune response without a harmful or otherwise unwanted associated immune responses or side effects. Also provided herein are methods for producing synthetic or recombinant multivalent coronavirus polypeptide immunogeris, including fusion proteins. For example, host cells containing an S protein immunogen-encoding nucleic acid expression construct may be cultured to produce the recombinant S protein immunogen, or variants thereof (e.g., deletion mutants or S polypeptide fragments lacking a C-terminal transmembrane domain). Also contemplated are methods for treating or preventing coronavirus infections or eliciting an immune response using an S protein immunogen or variant thereof, or a combination of polypeptides (including fusion proteins).

By way of background and not wishing to be bound by theory, coronavirus has a positive-sense, non-segmented, single-stranded RNA genome, which encodes at least 18 viral proteins (such as non-structural proteins (NSP) 1-13, structural proteins E, M, N, S), and an RNA-dependent RNA polymerase). Coronavirus has three major surface glycoproteins (designated S, E, and M), and some coronaviruses have another surface glycoprotein referred to as hemagglutinin esterase (HE), which is not found in the SARS virus. In addition, the N (nucleocapsid) protein is a basic phosphoprotein, which is generally associated with the genome and has been reported to be antigenic (Holmes and Lai, Fields Virology, Chapter 34, 1996). The S (spike) protein, a major antigen of coronavirus, has two domains: S1, which is believed to be involved in receptor binding and S2, believed to mediate membrane fusion between the virus and target cell (Holmes and Lai, 1996, supra).

The S (spike) protein may form non-covalently linked homotrimers (oligomers), which may mediate receptor binding and virus infectivity. Homotrimers of S proteins are likely necessary for presenting the correct native conformation of receptor binding domains and for eliciting a neutralizing antibody response. In addition, intracellular processing of S protein is associated with significant posttranslation oligosaccharide modification. The posttranslation oligosaccharide modification (glycosylation) expected by N-glycan motif analysis indicates that the S protein has as many as 23 sites for such modification. In addition, C-terminal cysteine residues may also participate in protein folding and preserving the native (functional) S protein conformation. The S protein of some coronaviruses (e.g., some strains of group II and III viruses) can be proteolytically processed near the center of the S protein by a trypsin-like protease in the Golgi apparatus or by extracellularly localized enzymes into to a linked polypeptide, containing an N-terminal S1 and a C-terminal S2 polypeptide. Some members of the type II group of coronaviruses and group I viruses may not be so processed. Until the characterization of the SARS-associated viral agent as a coronavirus, the coronaviruses were divided into three groups on the basis of serological and genetic properties, which groups were referred to as Group 1, Group 2, and Group 3, which are also referred to in the art and herein as Group I, Group II, and Group III (see, e.g., Holmes et al., Fields Virology, supra; Stadler et al., Nat. Rev. Microbiol. 209-18 (2003); Holmes, J. Clin. Invest. 111:1605-609 (2003)). Presently, the coronaviruses are subdivided into Group 1, Group 2, Group 3, and SARS-CoV (SARS-associated coronavirus) (see, e.g., Stadler et al., supra; Holmes, J. Clin. Invest., supra).

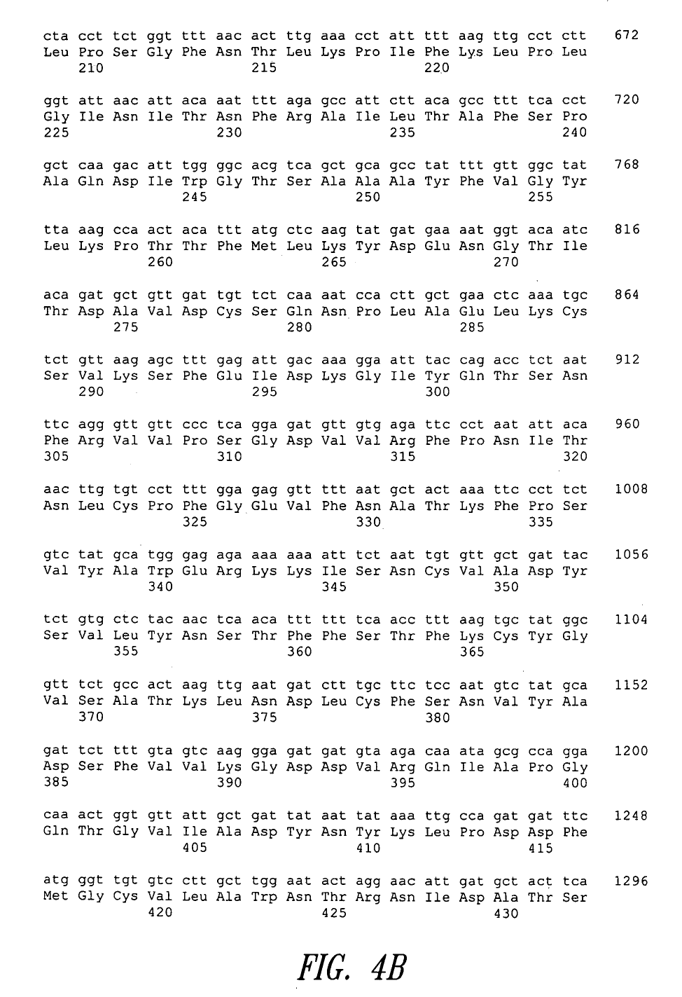

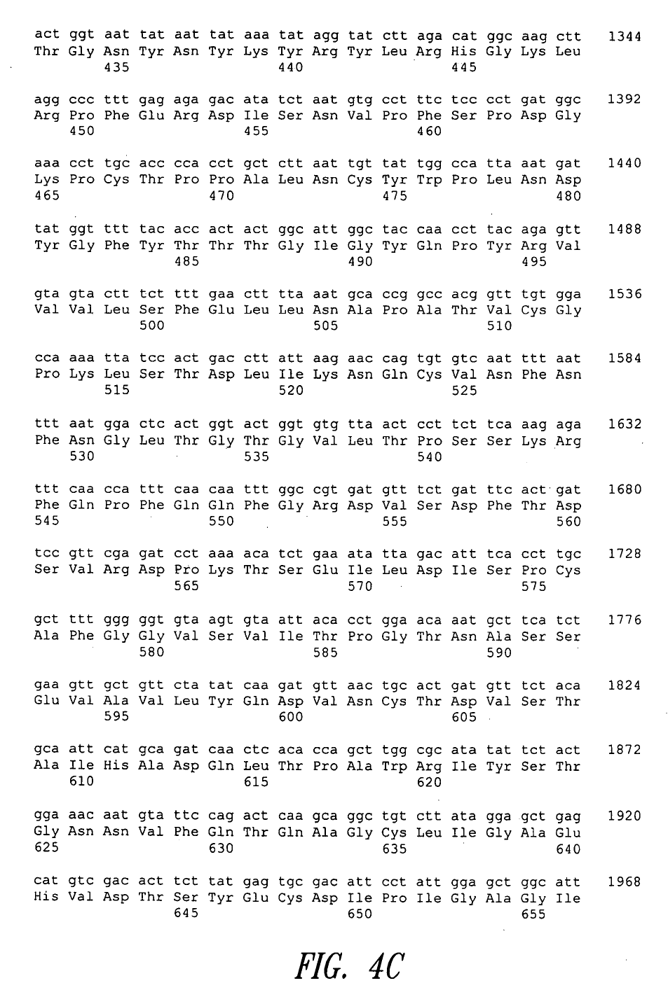

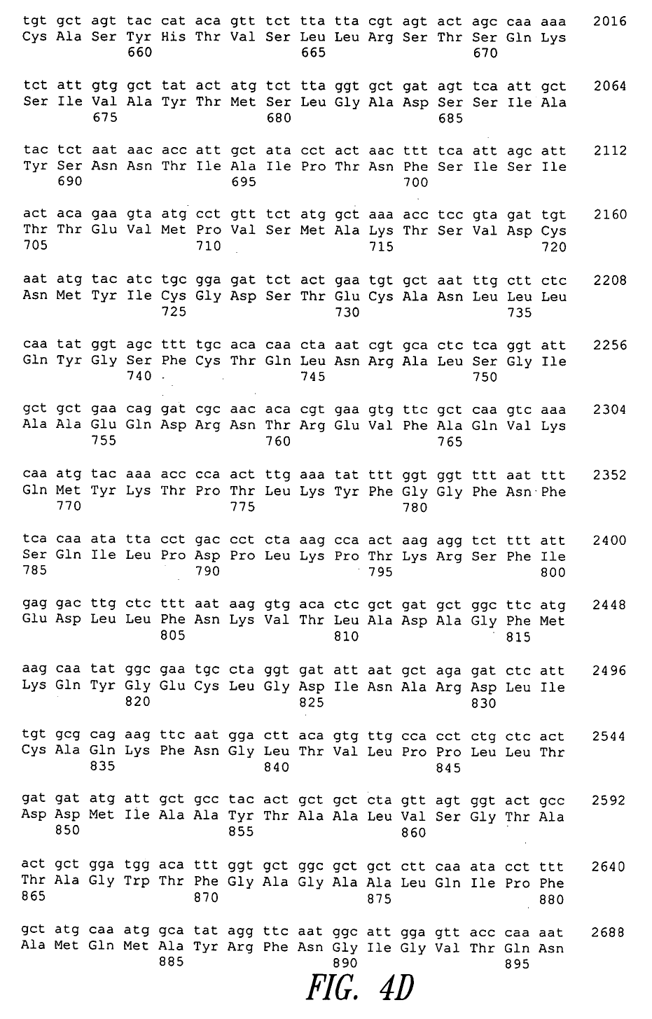

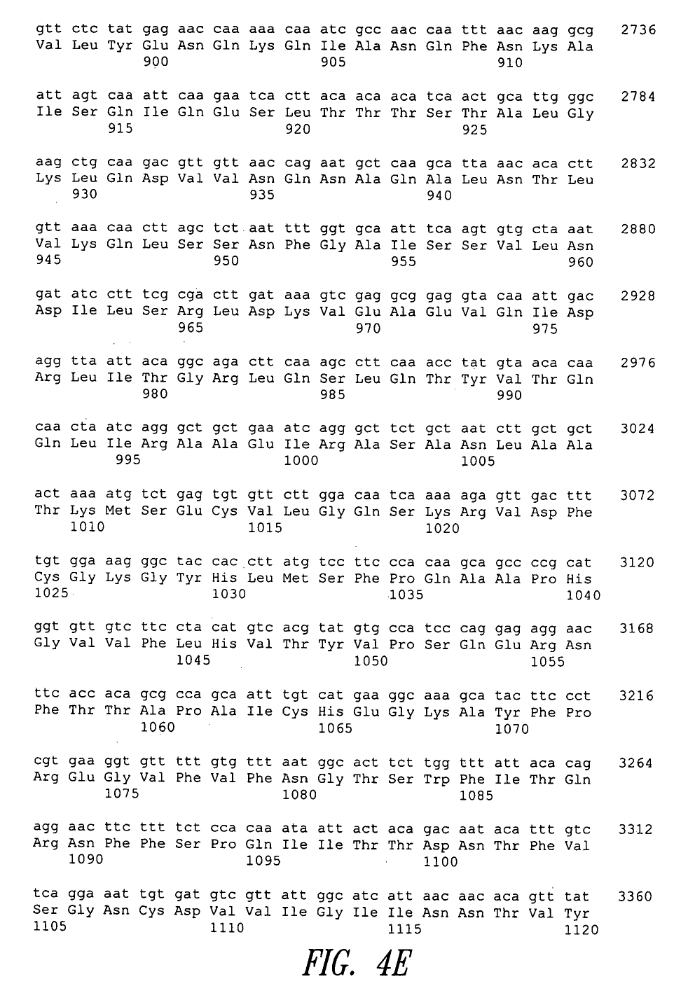

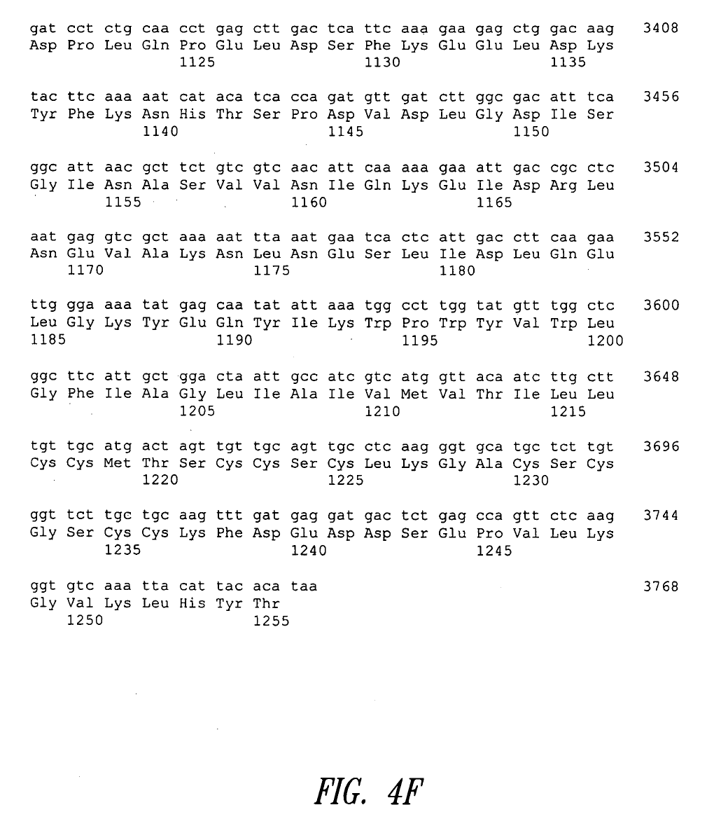

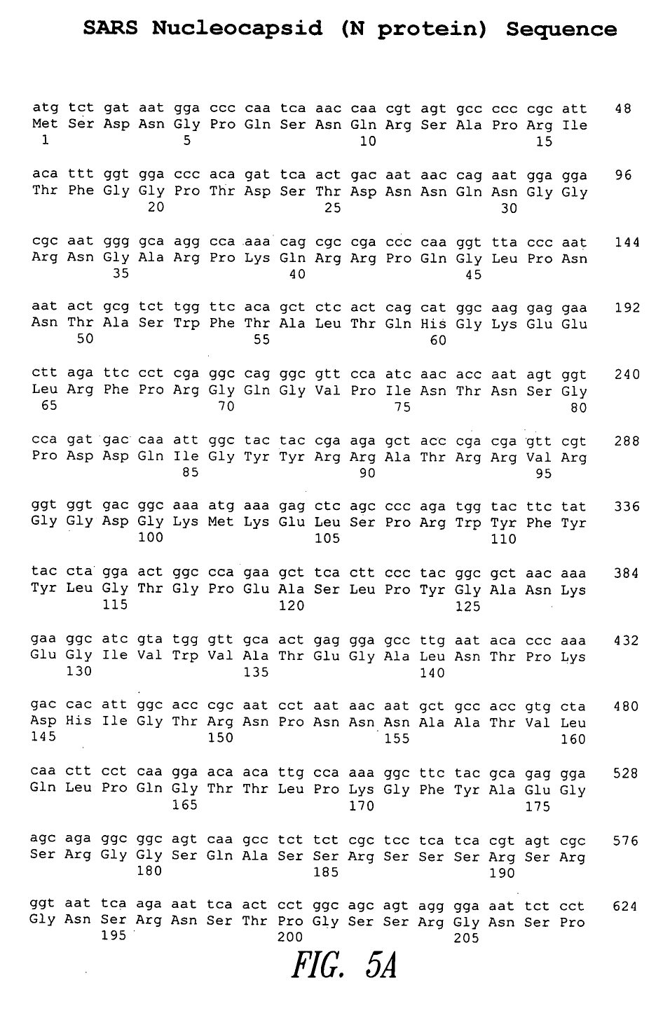

An exemplary SARS-CoV S protein has 1,255 amino acids (see, e.g., SEQ ID NO:2 and FIG. 4), with a 12 amino acid signal sequence, the S1 domain between amino acids 12-672 (see, e.g., SEQ ID NO:20), and the S2 domain between amino acids 673-1192 (see, e.g., SEQ ID NO:22). In certain embodiments, coronavirus S or N polypeptides and variants thereof that have one or more epitopes (i.e., are immunogens) and that are capable of eliciting a neutralizing (e.g., IgA or IgG antibody) or cell-mediated immune response, are included in compositions for use in treating or preventing coronavirus infections. Also described herein is the identification of S protein immunogens (containing one or more immunogenic epitopes) that are not glycosylated and that are capable of eliciting a neutralizing immune response. In one embodiment, the S protein immunogen is a portion or fragment of the full-length S protein. For example, a portion of the S protein immunogen that includes amino acids at positions 417-560 of SEQ ID NO:2 does not contain an N-glycan substitution site and is a hydrophilic region. This region also corresponds to the region of the S1 domain that is believed to be involved with cell receptor binding. Accordingly, a fragment comprising amino acids at positions 417-560 of SEQ ID NO:2, or a portion thereof (e.g., SEQ ID NO:12 and SEQ ID NO:14), may be immunogenic and an immune response specific for one or more epitopes within this sequence may prevent entry of the coronavirus into a target cell. In addition, identification of such immunogenic fragments of the S protein that do not contain glycosylation sites provides the advantage that the fragments may be made and produced in cells, such as bacteria, that are not capable of glycosylating a protein in the same manner as a mammalian cell. It is also important to note that vaccinia virus expressed S-protein from feline infectious peritonitis virus (FIPV) has been implicated in antibody-induced enhancement (ADE) of the virus infection (Vennema et al., Adv. Exp. Med. Biol. 276:217 (1990); Klepfer et al., Adv. Exp. Med. Biol. 380:235 (1995)). Therefore, in view of this description in the art, the capability of an S protein immunogen to elicit an immune response in a host as described herein, and thus provide advantages as a vaccine, may not be expected.

As described herein, an S protein immunogen includes a fragment of S protein or a S protein variant (which may be a variant of a full-length S protein or S fragment as described herein) that retains or that has at least one epitope contained within the full-length S protein or wildtype S protein, respectively, that elicits a protective immune response against coronavirus, preferably against SARS coronavirus. An S protein fragment or an S protein variant has at least one biological activity or function of a full-length or wildtype (natural) S protein (such as receptor binding or cell fusion activity), or has multiple S protein-specific biological activities or functions. For example, an S protein variant may contain an epitope that induces an immune response (for example, induces production of an antibody that specifically binds to a wildtype or full-length S polypeptide) or may have S protein receptor binding activity. In one embodiment, an S-protein fragment is a truncated S-protein that comprises an amino acid set forth at positions 1-1200 of SEQ ID NO:2 (SEQ ID NO:4). The portion of the S-protein that is deleted is the transmembrane region; the remaining fragment is also referred to herein as S_{TM-del} or ΔTM S-protein. In certain other embodiments, exemplary S protein fragments include an amino acid sequence set forth at positions 12-254 of SEQ ID NO:2 (SEQ ID NO:6); or at positions 255-834 of SEQ ID NO:2 (SEQ ID NO:8); or at positions 835-1255 of SEQ ID NO:2 (SEQ ID NO:10); or at positions 12-672 of SEQ ID NO:2 (SEQ ID NO:20; S1 domain); or at positions 673-1195 of SEQ ID NO:2 (SEQ ID NO:22; S2 domain). In certain other embodiments, an S polypeptide fragment includes an amino acid sequence set forth at positions 300-550 of SEQ ID NO:2 (SEQ ID NO:12); or at positions set forth at positions 380-580 of SEQ ID NO:2 (SEQ ID NO:14); or at positions 380-480 of SEQ ID NO:2 (SEQ ID NO:16); or at positions 481-580 of SEQ ID NO:2 (SEQ ID NO:18); or at positions 673-960 of SEQ ID NO:2 (SEQ ID NO:24); or at positions 961-1200 of SEQ ID NO:2 (SEQ ID NO:26). S protein immunogenic fragments also include smaller portions or fragments of the aforementioned amino acid fragments of an S protein. An S protein fragment that comprises an epitope that stimulates, induces, or elicits an immune response may comprise a sequence of consecutive amino acids ranging from any number of amino acids between 8 amino acids and 150 amino acids (e.g., 8, 10, 12, 15, 18, 20, 25, 30, 35, 40, 50, etc. amino acids) of any one of SEQ ID NOS:2, 4, 6, 8, 10, 12, 14, 16, 18, 20, 22, 24, or 26.

In related embodiments, a coronavirus S polypeptide variant has at least 50% to 100% amino acid identity (that is, at least 50%, 55%, 60%, 65%, 70%, 75%, 80%, 85%, 90%, 95%, or 99% identity) to the amino acid sequence of the full length S protein as set forth in SEQ ID NO:2 (which is from SARS-CoV Tor2 strain; SEQ ID NO:1 is the nucleic acid sequence that encodes the amino acid sequence of SEQ ID NO:2), or 50% to 100% amino acid identity (that is, at least 50%, 55%, 60%, 65%, 70%, 75%, 80%, 85%, 90%, 95%, or 99% identity) to an S protein fragment as set forth in any one of SEQ ID NOS:4, 6, 8, 10, 12, 14, 16, 18, 20, 22, 24, and 26. Such S polypeptide variants and fragments retain at least one S protein-specific biological activity or function, such as (1) the capability to elicit a protective immune response (that is, the S polypeptide variant contains an epitope that induces or elicits a protective immune response), for example, a neutralizing response and/or a cell-mediated immune response against coronavirus, such as SARS-CoV; (2) the capability to mediate viral infection via receptor binding; and (3) the capability to mediate membrane fusion between a virion and the host cell.

Additional examples of full-length SARS coronavirus S (spike) polypeptide sequences are provided herein and available in the art. For example, a full-length S protein of the SARS coronavirus Frankfurt 1 strain is provided in SEQ ID NO:45, which is encoded by the polynucleotide sequence set forth in SEQ ID NO:44. A full-length S protein of the SARS coronavirus TW5 strain is provided in SEQ ID NO:47, which is encoded by the polynucleotide sequence set forth in SEQ ID NO:46. A full-length S protein of the SARS coronavirus GD03T0013 strain is provided in SEQ ID NO:49, which is encoded by the polynucleotide sequence set forth in SEQ ID NO:48. A full-length S protein of the SARS coronavirus BJ01 strain is provided in SEQ ID NO:51, which is encoded by the polynucleotide sequence set forth in SEQ ID NO:50. In certain embodiments, fragments (such as truncated S protein that has a deletion of the transmembrane domain) and variants of any one of these full-length S polypeptides may be used as immunogens for eliciting a protective immune response against a coronavirus, particularly a SARS coronavirus.

In other embodiments, a fragment of N protein, or a variant of a fragment or a variant of the full-length N protein, includes an immunogen that retains or has at least one N protein related biological activity or function, such as (1) the capability to induce an immune response (that is, the N polypeptide variant contains an epitope that induces or elicits an immune response), which may be, for example, a humoral response (i.e., eliciting production of an antibody that specifically binds to a wildtype or full-length N polypeptide) and/or a cell-mediated immune response against coronavirus, such as SARS-CoV; (2) the capability to bind to a nucleic acid, such as RNA; and (3) the capability to promote pathogenesis of the coronavirus (see, e.g., Chen et al. Clin. Chem. 50:988-95 (2004) (describing an amino acid sequence located in the N protein that contained a motif related to a nuclear localization signal)).

Also described herein are other N proteins and variants thereof having at least one N protein related biological activity (such as nucleic acid binding activity or the ability to specifically bind to an antibody that specifically binds to N protein). In certain embodiments, exemplary N protein immunogen fragments and variants thereof include a fragment having an amino acid sequence at positions 1-211 of SEQ ID NO:28 (SEQ ID NO:30); or at positions 212-422 of SEQ ID NO:28 (SEQ ID NO:32); or at positions 100-300 of SEQ ID NO:28 (SEQ ID NO:34). In other embodiments, an N polypeptide fragment includes an amino acid sequence at positions 50-250 of SEQ ID NO:28 (SEQ ID NO:36); or at positions 150-400 of SEQ ID NO:28 (SEQ ID NO:38). N protein immunogenic fragments also include smaller portions or fragments of the aforementioned amino acid fragments of an N protein. An N protein fragment that comprises an epitope that stimulates or elicits an immune response may comprise a sequence of consecutive amino acids ranging from any number of amino acids between 8 amino acids and 150 amino acids (e.g., 8, 10, 12, 15, 18, 20, 25, 30, 35, 40, 50, etc. amino acids) of any one of SEQ ID NO: 28, 30, 32, 34, 36, and 38.

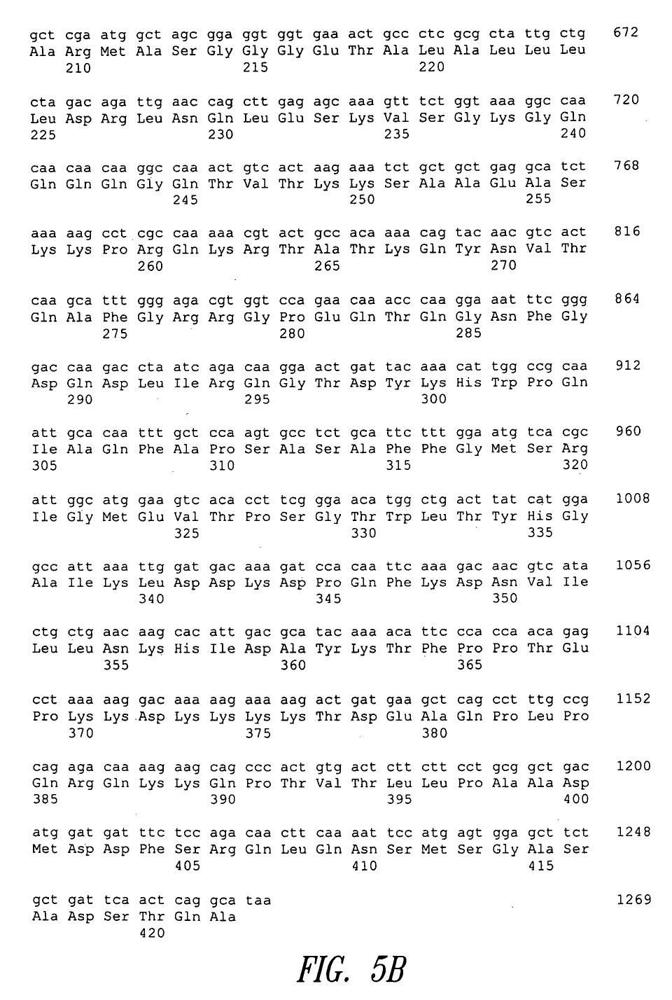

Variants of the N polypeptide or fragments of the full-length N protein or variants thereof have at least 50%, 55%, 60%, 65%, 70%, 75%, 80%, 85%, 90%, 95%, or 99% identity to the amino acid sequences as set forth in any one of SEQ ID NOS:28, 30, 32, 34, 36, and 38. As described herein, an N polypeptide variant retains at least one N protein-specific activity, such as the capability to elicit a protective humoral or cell-mediated immune response against coronavirus, such as SARS-CoV, or at least one other N protein related biological activity, such as nucleic acid binding activity. In a related embodiment, the coronavirus N polypeptides have at least %, 55%, 60%, 65%, 70%, 75%, 80%, 85%, 90%, 95%, or 99% amino acid identity to an amino acid sequence of the full length N protein as set forth in SEQ ID NO:28 (from SARS-CoV Urbani strain, see FIG. 5; see also SEQ ID NO:27 that sets forth the nucleotide sequence that encodes the amino acid sequence of SEQ ID NO:28).

Additional examples of full-length SARS coronavirus N (nucleocapsid) polypeptide sequences are provided herein and available in the art. For example, a full-length N protein of the SARS coronavirus HB strain is provided in SEQ ID NO:55, which is encoded by the polynucleotide sequence set forth in SEQ ID NO:54.

Nucleotide sequences and amino acid sequences of two or more coronavirus polynucleotides and polypeptides and variants thereof, respectively, can be compared using any standard software program, such as BLAST, tBLAST, pBLAST, or MegAlign. Still others include those provided in the Lasergene bioinformatics computing suite, which is produced by DNASTAR® (Madison, Wis.). References for algorithms such as ALIGN or BLAST may be found in, for example, Altschul, J. Mol. Biol. 219:555-565, 1991; or Henikoff and Henikoff, Proc. Natl. Acad. Sci. USA 89:10915-10919, 1992. BLAST is available at the NCBI website. Other methods for comparing multiple nucleotide or amino acid sequences by determining optimal alignment are well known to those of skill in the art (see, e.g., Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology, pages 123-151 (CRC Press, Inc. 1997); and Bishop (ed.), Guide to Human Genome Computing, 2nd edition, Academic Press, Inc., 1998).

As used herein, “percent identity” or “% identity” is the percentage value returned by comparing the whole of the subject polypeptide, peptide, or variant thereof sequence to a test sequence using a computer implemented algorithm, typically with default parameters. The variant polypeptides and immunogens described herein could be made to include one or more of a variety of mutations, such as point mutations, frameshift mutations, missense mutations, additions, deletions, and the like, or the variants can be a result of modifications, such as by certain chemical substituents, including glycosylation, alkylation, etc. As used herein, “similarity” between two peptides or polypeptides is generally determined by comparing the amino acid sequence of one peptide or polypeptide to the amino acid sequence and conserved amino acid substitutes thereto of a second peptide or polypeptide.

As described herein, S or N protein immunogens, fragments, and variants thereof described herein contain an epitope that elicits or induces an immune response, preferably a protective immune response, which may be a humoral response and/or a cell-mediated immune response. A protective immune response may be manifested by at least one of the following: preventing infection of a host by a coronavirus; modifying or limiting the infection; aiding, improving, enhancing, or stimulating recovery of the host from infection; and generating immunological memory that will prevent or limit a subsequent infection by a coronavirus. A humoral response may include production of antibodies that neutralize infectivity, lyse the virus and/or infected cell, facilitate removal of the virus by host cells (for example, facilitate phagocytosis), and/or bind to and facilitate removal of viral antigenic material. A humoral response may also include a mucosal response, which comprises eliciting or inducing a specific mucosal IgA response.

Induction of an immune response in a subject or host (human or non-human animal) by a coronavirus polypeptide, fragment, or variant described herein, may be determined and characterized by methods described herein and routinely practiced in the art. These methods include in vivo assays, such as animal immunization studies (e.g., using a rabbit, mouse, ferret, civet cat, African green monkey, or rhesus macaque model), and any one of a number of in vitro assays, such as immunochemistry methods for detection and analysis of antibodies, including Western immunoblot analysis, ELISA, immunoprecipitation, radioimmunoassay, and the like, and combinations thereof. By way of example, animal models may be used for determining the capability of a coronavirus antigen to elicit and induce an immune response that is protective in animals, which may be determined by endpoints relevant to the particular model. An example of an animal model to study SARS in cynomologous macaques is described in Kuiken et al., Lancet 362:263-70 (2003). Cat and ferret animal models may also be useful for studying SARS (see, e.g., Martina et al., Nature 425:915 (2003)).

Other methods and techniques that may be used to analyze and characterize an immune response include neutralization assays (such as a plaque reduction assay or an assay that measures cytopathic effect (CPE) or any other neutralization assay practiced by persons skilled in the art) to assess whether an S or N protein immunogen or variant thereof is capable of eliciting an immune response, particularly a neutralizing immune response (see, e.g., Schmidt et al., Diagnostic Procedures for Viral, Rickettsial and Chlamydial Infections, 6^{th} ed., American Public Health Association (Washington 1989); Marra et al., Science 300:1399-404 (2003); Guo et al., Virology 324:251-56 (2004)). Briefly, an animal is immunized with an S or N protein immunogen or a composition containing at least one S protein immunogen or at least one N protein immunogen, or a cocktail composition comprising at least one S protein immunogen and at least one N protein immunogen, by subcutaneous, intraperitoneal, intranasal, intravenus or other appropriate administration described herein and practiced by persons skilled in the art. Sera are collected from immunized animals and tested for the capability of antibodies present in the sera to inhibit coronavirus infection of a cell culture monolayer (for example, infection may be measured by the number of plaques (i.e., “holes”) in the monolayer arising from coronavirus causing cells to lyse (plaque reduction assay) or by determining microscopically the cytopathic effect in a CPE assay). In addition, an immune response that is elicited or induced (i.e., has changed or altered compared with the immune response prior to immunization) may be identified and characterized by determining cytokine expression patterns in animals challenged with coronavirus after immunization with one or more S or N protein immunogens described herein according to methods described herein and known to persons skilled in the art. For example, specific cytokine levels can be measured in tissues of interest by using a ribonuclease protection assay (RPA) to determine whether a type 1 or type 2 response is prevalent after immunization with an S or N protein immunogen and subsequent challenge with coronavirus. Another exemplary assay is an ELISA using OptEIA kits (BD Biosciences, San Jose, Calif.) to measure the level of one or more cytokines.

In vitro assays useful for detecting and characterizing coronavirus polypeptides and variants thereof (e.g., S protein variants or N protein variants) that retain a biological activity include, for example, competitive ELISA techniques or competitive receptor binding techniques, which may be used to identify the presence of and/or determine the function (biological activity) of the coronavirus polypeptides and variants. A coronavirus antigen (such as at least one S protein immunogen, one N protein immunogen, or at least one of each of an S and N protein immunogen that may be combined (mixed, admixed, or formulated) in a physiological excipient and/or a Proteosome-based composition as described herein, including Protollin) may evoke (induce the production of or elicit) a neutralizing antibody response that is dependent upon the presentation of an epitope present in the native coronavirus polypeptide. The presence of a conformational or sequential epitope may be determined, for example, by protein binding assays, which may include using a monoclonal antibody, or a competitive binding assay format using an antibody known to specifically bind the antigen or using a ligand (e.g., coronavirus protein, S or N protein or fragment thereof), or receptor.

A native polypeptide (or protein) herein refers to a coronavirus protein in its native conformation as it is found in an assembled virus or during assembly of the virus, that is, the protein has adopted its native topographical structure. The native conformation may also be adopted by a recombinantly expressed coronavirus polypeptide. An epitope (also referred to herein and in the art as an antigenic determinant) that induces a humoral response, that is, an antibody response, may be conformational, and thus, as found in a native coronavirus protein. Alternatively, the epitope or antigenic determinant may be sequential, that is the epitope comprises consecutive amino acids of one or more of the coronavirus protein sequences described herein. A humoral immune response may be induced by a conformational or a sequential epitope or by a combination of epitopes. A cell-mediated response that includes T cell recognition may depend on presentation of a processed coronavirus protein fragment (or fragments) that retains only the primary and secondary structure (i.e., sequential epitope).

These and other assays and methods known in the art can be used to identify and characterize S or N protein immunogens and variants thereof that have at least one epitope that elicits a protective humoral or cell-mediated immune response against coronavirus. The statistical significance of the results obtained in the various assays may be calculated and understood according to methods routinely practiced by persons skilled in the relevant art.

The coronavirus S or N protein immunogens (full-length proteins, variants, fragments, and fusion proteins thereof), as well as corresponding nucleic acids encoding such immunogens, are provided in an isolated form, and in certain embodiments, are purified to homogeneity. As used herein, the term “isolated” means that the nucleic acid or polypeptide is removed from its original or natural environment. For example, a naturally occurring nucleic acid molecule or polypeptide encoded by the nucleic acid present in a living animal or cell is not isolated, but the same nucleic acid molecule or polypeptide is isolated when separated from some or all of the co-existing materials in the natural system. The nucleic acid molecules, for example, could be part of a vector, and/or such nucleic acids or polypeptides could be part of a composition and still be isolated in that such vector or composition is not part of the natural environment of the nucleic acid molecule or the polypeptide.

A coronavirus S or N protein immunogen (and corresponding immunogenic epitopes) and fragments, and variants thereof may be produced synthetically or recombinantly. A coronavirus protein fragment that contains an epitope that induces an immune response against coronavirus may be synthesized by standard chemical methods, including synthesis by automated procedure. In general, immunogenic peptides are synthesized based on the standard solid-phase Fmoc protection strategy with HATU as the coupling agent. The immunogenic peptide is cleaved from the solid-phase resin with trifluoroacetic acid containing appropriate scavengers, which also deprotects side chain functional groups. The crude immunogenic peptide may be further purified using preparative reverse phase chromatography. Other purification methods, such as partition chromatography, gel filtration, gel electrophoresis, or ion-exchange chromatography may be used. Other synthesis techniques known in the art may be employed to produce similar immunogenic peptides, such as the tBoc protection strategy, use of different coupling reagents, and the like. In addition, any naturally occurring amino acid or derivative thereof may be used, including D-amino acids or L-amino acids, and combinations thereof. In certain embodiments, a synthetic S protein immunogen has an amino acid sequence that is identical to, or at least 80% identical (which includes at least 85%, 90%, or 95% or any percent in between 80% and 100%) to SEQ ID NOS:2, 4, 6, 8, 10, 12, 14, 16, 18, 20, 22, 24, or 26. In other embodiments, a synthetic N protein immunogen of the invention will have an amino acid sequence that is identical to or at least 80% identical (which includes at least 85%, 90%, or 95% or any percent in between 80% and 100%) to SEQ ID NOS:28, 30, 32, 34, 36 or 38.

As described herein, the S or N protein immunogens may be recombinant, wherein desired S or N protein immunogens are individually or in combination expressed from a polynucleotide thatis operably linked to an expression control sequence (e.g., a promoter) in a nucleic acid expression construct. In certain embodiments, a recombinant S protein antigen will comprise an amino acid sequence that is identical to, or at least 80% identical (which includes at least 85%, 90%, or 95% or any percent in between 80% and 100%) to SEQ ID NO:2. In another embodiment, a recombinant S protein immunogen consists of an amino acid sequence as set forth in SEQ ID NO:2. In other embodiments, recombinant S protein immunogens and variants thereof are fragments of SEQ ID NO:2, which can comprise an amino acid sequence of SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26 or sequences that are identical to, or at least 80% identical (which includes at least 85%, 90%, or 95% or any percent in between 80% and 100%) to each of the aforementioned amino acid sequences. In certain other embodiments, a recombinant N protein immunogen and variant thereof comprises an amino acid sequence set forth in SEQ ID NO:28, or is a variant thereof, or comprises a fragment of SEQ ID NO:28, which can comprise an amino acid sequence of SEQ ID NO:30, SEQ ID NO:32, SEQ ID NO:34, SEQ ID NO:36, and SEQ ID NO:38, or that are identical to, or at least 80% identical (which includes at least 85%, 90%, or 95% or any percent in between 80% and 100%) to each of these amino acid sequences.

A polynucleotide, nucleic acid, or nucleic acid molecule refers to any of single-stranded or double-stranded deoxyribonucleic acid (DNA) or ribonucleic acid (RNA) polynucleotide, oligonucleotide, or fragment thereof. Polynucleotides may be isolated from a biological source and/or may be amplified and generated by the polymerase chain reaction (PCR). Polynucleotide fragments may be obtained from a PCR product or from an isolated polynucleotide by any of ligation, scission, endonuclease, and/or exonuclease activity. Nucleic acids may be composed of monomers that are naturally occurring nucleotides (such as deoxyribonucleotides and ribonucleotides), analogs of naturally occurring nucleotides (e.g., α-enantiorheric forms of naturally-occurring nucleotides), or a combination of both. Modified nucleotides can have modifications in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety may be replaced with sterically and electronically similar structures, such as aza-sugars and carbocyclic sugar analogs. Examples of modifications of a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid” also includes “peptide nucleic acids,” which comprise naturally occurring or modified nucleic acid bases attached to a polyamide backbone.

Further, an isolated nucleic acid molecule refers to a nucleic acid molecule (polynucleotide or nucleic acid) in the form of a separate fragment, or as a component of a larger nucleic acid construct, which has been separated from its source cell (including the chromosome it normally resides in if applicable) or virus in a substantially pure forth. For example, a DNA molecule that encodes a coronavirus polypeptide, peptide, or variant thereof, which has been separated from a coronavirus particle or from a host cell infected with or harboring coronavirus, is an isolated DNA molecule. Another example of an isolated nucleic acid molecule is a chemically synthesized nucleic acid molecule. Nucleic acid molecules may be comprised of a wide variety of nucleotides, including DNA, cDNA, RNA, nucleotide analogues, or some combination thereof.

In one embodiment, an isolated nucleic acid molecule comprises a sequence encoding an S protein immunogen comprising an amino acid sequence that is identical to or at least 80% identical (which includes at least 85%, 90%, or 95% or any percent in between 80% and 100%) to SEQ ID NO:2. In certain embodiments, the nucleic acid molecule encodes an S protein immunogen that has an antigenic epitope that elicits a protective immune response, which includes a humoral response (e.g., elicitation and production of mucosal IgA and/or systemic IgG or IgM or IgA) and/or a cell-mediated immune response against coronavirus. In another embodiment, an isolated nucleic acid molecule comprises a sequence encoding an S protein immunogen that has an amino acid sequence consisting of SEQ ID NO:2. In still other embodiments, an isolated nucleic acid molecule encodes an S protein immunogen fragment of SEQ ID NO:2, which fragment may comprise an amino acid sequence that is identical to or at least 80% identical to (which includes at least 85%, 90%, or 95% or any percent in between 80% and 100%) an amino acid sequence selected from SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26. In other embodiments, an isolated nucleic acid molecule comprises a sequence that encodes an N protein immunogen that has an amino acid sequence comprising or consisting of SEQ ID NO:28, or a variant thereof. In another embodiment, an isolated nucleic acid molecule encodes an N protein immunogen fragment of SEQ ID NO:28, which fragment can comprise an amino acid sequence that is identical to or at least 80% identical to (which includes at least 85%, 90%, or 95% or any percent in between 80% and 100%) an amino acid sequence set forth in any one of SEQ ID NO:30, SEQ ID NO:32, SEQ ID NO:34, SEQ ID NO:36, and SEQ ID NO:38.

Also provided herein are nucleic acid vectors and constructs that include nucleotide sequences that encode coronavirus immunogens, and in particular to nucleic acid expression constructs (also called recombinant expression constructs) that include any polynucleotide encoding a coronavirus polypeptide or fragment, or variant thereof, as described herein, and regulatory nucleotide sequences. Host cells may be genetically engineered to comprise such vectors or constructs, which host cells may be produced and used in methods for treating or preventing a coronavirus infection or eliciting an immune response against a coronavirus infection. The coronavirus polypeptides and fragments or variants thereof may be expressed in mammalian cells, yeast, bacteria, or other cells (e.g., insect cells) under the control of appropriate expression control sequences, including a promoter sequence. Cell-free translation systems may also be employed to produce such coronavirus proteins using nucleic acids, including RNAs, and expression constructs. Appropriate cloning and expression vectors for use with prokaryotic and eukaryotic hosts are routinely used by persons skilled in the art and are described, for example, by Sambrook et al., Molecular Cloning: A Laboratory Manual, Second Edition, Cold Spring Harbor, N.Y., (1989) and Third Edition (2001), and may include plasmids, cosmids, shuttle vectors, viral vectors, and vectors comprising a chromosomal origin of replication as disclosed therein.

In one embodiment, a nucleic acid expression construct comprises an expression control sequence, such as a promoter, operably linked to a polynucleotide encoding an S protein immunogen or variant thereof comprising an amino acid sequence that is identical to or at least 80% identical to (which includes at least 85%, 90%, or 95% or any percent in between 80% and 100%) SEQ ID NO:2, wherein the S protein immunogen has at least one epitope that elicits a humoral response (e.g., including a neutralizing antibody) and/or cell-mediated immune response against coronavirus infection, such as a SARS coronavirus infection. In certain embodiments, a nucleic acid expression construct comprises an expression control sequence operably linked to a polynucleotide encoding an S protein immunogen that has an amino acid sequence consisting of SEQ ID NO:2. In other embodiments, a nucleic acid expression construct comprises an expression control sequence such as a promoter sequence operably linked to at least one polynucleotide encoding at least one S protein immunogen or variant thereof that is a fragment of SEQ ID NO:2, which comprises an amino acid sequence that is identical to or at least 80% identical to (which includes at least 85%, 90%, or 95% or any percent in between 80% and 100%) an amino acid sequence selected from SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26.

In still other embodiments, a nucleic acid expression construct comprises an expression control sequence, such as a promoter, operably linked to at least one polynucleotide encoding at least one N protein immunogen or variant thereof or that is a fragment of an N protein immunogen or variant thereof and has an amino acid sequence that is identical to or at least 80% identical to (which includes at least 85%, 90%, or 95% or any percent in between 80% and 100%) SEQ ID NO:28, SEQ ID NO:30, SEQ ID NO:32, SEQ ID NO:34, SEQ ID NO:36, or SEQ ID NO:38. In a related embodiment, a nucleic acid expression construct comprises an expression control sequence, such as a promoter, operably linked to a polynucleotide encoding such an N protein immunogen or variant thereof, wherein the N protein immunogen has an epitope that elicits a humoral response (e.g., including a neutralizing antibody) and/or cell-mediated immune response against a coronavirus infection, for example, a SARS coronavirus infection.

As will be appreciated by those of ordinary skill in the art, a nucleotide sequence encoding a coronavirus polypeptide or variant thereof may differ from the sequences presented herein due to, for example, the degeneracy of the genetic code. A nucleotide sequence that encodes a coronavirus polypeptide variant includes a sequence that encodes a homolog or strain variant or other variant. Variants may result from natural polymorphisms or may be synthesized by recombinant methodology (e.g., to obtain codon optimization for expression in a particular host or to introduce an amino acid mutation) or chemical synthesis, and may differ from wild-type polypeptides by one or more amino acid substitutions, insertions, deletions, and the like. A polynucleotide variant that encodes a coronavirus polypeptide variant encompasses a polynucleotide preferably encodes conservative amino acid substitutions. Examples of conservative substitutions include substituting one aliphatic amino acid for another, such as Ile, Val, Leu, or Ala, or substituting one polar residue for another, such as between Lys and Arg, Glu and Asp, or Gln and Asn. A similar amino acid or a conservative amino acid substitution is also one in which an amino acid residue is replaced with an amino acid residue having a similar side chain, which include amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, histidine); nonpolar side chains (e.g., alanine, valine, leucine, isoleucine, proline, phenylalanine, methionine, tryptophan); beta-branched side chains (e.g., threonine, valine, isoleucine), and aromatic side chains (e.g., tyrosine, phenylalanine, tryptophan). Proline, which is considered more difficult to classify, shares properties with amino acids that have aliphatic side chains (e.g., Leu, Val, Ile, and Ala). In certain circumstances, substitution of glutamine for glutamic acid or asparagine for aspartic acid may be considered a similar substitution in that glutamine and asparagine are amide derivatives of glutamic acid and aspartic acid, respectively.

Conservative and similar substitutions of amino acids in the coronavirus immunogen sequences disclosed herein may be readily prepared according to methods described herein and practiced in the art and which provide variants retaining similar physical properties and functional or biological activities, such as, for example, the capability to induce or elicit an immune response, which may include a humoral response (that is, eliciting antibodies that bind to and have the same biological activity as an antibody that specifically binds to the wildtype (or nonvariant) immunogen and/or that binds to antibodies that specifically bind to the wildtype or nonvariant immunogen). An S protein immunogen variant thereof preferably retains the capability to bind to cellular receptors and to mediate infectivity. An N protein immunogen and variant thereof retains, for example, the capability to complex with or bind to nucleic acids.

Certain variants include nucleic acid sequences that encode an S protein immunogen having at least 50% to 100% or greater than 90% or 95% identity or that is identical to or at least 80% identical to (which includes at least 85%, 90%, or 95% or any percent in between 80% and 100%) the amino acid sequence set forth in one or more of SEQ ID NOS:2, 4, 6, 8, 10, 12, 14, 16, 18, 20, 22, 24, or 26. Certain other variants include nucleic acid sequences that encode an N protein immunogen having at least 50% to 100% or greater than 90% or 95% identity or that is identical to or at least 80% identical to (which includes at least 85%, 90%, or 95% or any percent in between 80% and 100%) the amino acid sequence set forth in one or more of SEQ ID NOS:28, 30, 32, 34, 36, or 38. Polynucleotide variants also include all degenerate nucleic acid molecules that encode an S protein immunogen comprising an amino acid sequence set forth in SEQ ID NOS:2, 4, 6, 8, 10, 12, 14, 16, 18, 20, 22, 24, or 26, or a variant thereof. In another embodiment, polynucleotide variants include all degenerate nucleic acid molecules that encode an N protein immunogen comprising an amino acid sequence set forth in SEQ ID NOS:28, 30, 32, 34, 36, or 38 or a variant thereof.

As described herein a variant of an S protein immunogen retains at least one biological or functional activity such as having the capability to elicit or induce a protective immune response that may include a humoral (including a mucosal IgA response) and/or or cell-mediated immune response. Also as described herein a coronavirus S polypeptide variant may also have a biological activity substantially similar to that of the native or wildtype S protein such as the capability to specifically bind to an S protein antibody that is a neutralizing antibody (i.e., neutralizes viral infectivity); the capability to elicit or induce the production of an antibody that specifically binds to S protein; the capability to elicit or induce the production of an antibody that has the capability to neutralize virus infection; the capability to elicit or induce a cell-mediated immune response; and/or the capability to bind to an S protein cellular receptor. As described herein an N protein immunogen variant retains at least one biological or functional activity such as having the capability to elicit or induce a protective immune response that may include a humoral (including a mucosal IgA response) and/or cell-mediated immune response. Also as described herein a coronavirus N protein variant retains at least one biological activity substantially similar to that of the native N protein such as the capability to bind to an N protein specific antibody that is a protective antibody, for example, a neutralizing antibody, the capability to elicit or induce the production of an antibody that specifically binds to an N protein immunogen, the capability to elicit or induce an immune response (humoral and/or cell-mediated), or the capability to bind to a nucleic acid molecule such as the coronavirus genomic RNA. As described herein nucleic acid molecule variants encode S or N polypeptide derivatives or variants that have conservative amino acid substitutions such that the coronavirus polypeptide variants retain or have at least one epitope (from wild-type S or N polypeptide, respectively) capable of eliciting antibodies specific for one or more coronavirus strains, and/or that retain at least one biological activity of an S or N protein, respectively.

In certain embodiments, a nucleic acid sequence may be modified to encode a coronavirus S or N protein fragment or functional variant thereof wherein specific codons of the nucleic acid sequence have been changed to codons that are favored by a particular host and can result in enhanced levels of expression (see, e.g., Haas et al., Curr. Biol. 6:315, 1996; Yang et al., Nucleic Acids Res. 24:4592, 1996). For example, certain codons of the immunogenic peptides or polypeptides can be optimized for improved expression in Escherichia coli without changing the primary sequence of the peptides. By way of illustration and not limitation, arginine (Arg) codons of AGG/AGA can be changed to the Arg codons of CGT/CGC. Similarly, AGG/AGA Arg codons can be changed to CGT/CGC codons. As understood in the art, codons may be optimized for the particular host in which the hybrid polypeptide is to be expressed, including bacteria, fungi, insect cells, plant cells, and mammalian cells. Additionally, codons encoding different amino acids may be changed as well, wherein one or more codons encoding different amino acids may be altered simultaneously as would best suit a particular host (e.g., codons for arginine, glycine, leucine, and serine may all be optimized, and any combination thereof). Alternatively, codon optimization may result in one or more changes in the primary amino acid sequence, such as a conservative amino acid substitution, addition, deletion, and combinations thereof.

As described herein, a polynucleotide that encodes a coronavirus S protein immunogen includes any one of the nucleotide sequences set forth in SEQ ID NOS:1, 3, 5, 7, 9, 11, 13, 15, 17, 19, 21, 23, or 25, which encode the S protein immunogens having the amino acid sequences set forth in SEQ ID NOS:2, 4, 6, 8, 10, 12, 14, 16, 18, 20, 22, 24, and 26, respectively. A variant polynucleotide that encodes a coronavirus S protein immunogen includes a polynucleotide that is at least 50%, 55%, 60%, 65%, 70%, 75%, 80%, 85%, 90%, 95%, or 99% identical to a nucleotide sequence set forth in SEQ ID NOS:1, 3, 5, 7, 9, 11, 13, 15, 17, 19, 21, 23, or 25. Such a variant polynucleotide may, because of the degeneracy of the genetic code, encode an S protein immunogen comprising an amino acid sequence set forth in SEQ ID NOS:2, 4, 6, 8, 10, 12, 14, 16, 18, 20, 22, 24 or 26, respectively, or may encode an S protein variant immunogen as described herein (a S protein variant immunogen retains at least one biological or functional activity such as the capability to specifically bind to an S protein antibody that is a neutralizing antibody (i.e., that neutralizes viral infectivity), to elicit or induce the production of an antibody that specifically binds to S protein, to elicit or induce the production of an antibody that has the capability to neutralize virus infection, to elicit or induce a cell-mediated immune response, and/or the capability to bind to an S protein cellular receptor). Thus in certain embodiments, isolated nucleic acids (or polynucleotides) includes variants of SEQ ID NOS:1, 3, 5, 7, 9, 11, 13, 15, 17, 19, 21, 23, or 25 that are substantially similar to these sequences in that the variant nucleotide sequences encode native or non-native coronavirus S polypeptides with similar structure and ability to elicit specific antibodies to at least one S protein epitope contained in the coronavirus S protein polypeptides of SEQ ID NOS:2, 4, 6, 8, 10, 12, 14, 16, 18, 20, 22, 24, or 26.

Also described herein are isolated nucleic acids that encode coronavirus N protein immunogens and examples of these nucleotide sequences are set forth in SEQ ID NOS: 27, 29, 31, 33, 35, and 37, which encode the polypeptides having the amino acids sequences set forth in SEQ ID NOS: 28, 30, 32, 34, 36, and 38, respectively. A variant polynucleotide that encodes a coronavirus N protein immunogen includes a polynucleotide that is at least 50%, 55%, 60%, 65%, 70%, 75%, 80%, 85%, 90%, 95%, or 99% identical to a nucleotide sequence set forth in SEQ ID NOS: 27, 29, 31, 33, 35, and 37. Such a variant polynucleotide may, because of the degeneracy of the genetic code, encode an N protein immunogen comprising an amino acid sequence set forth in SEQ ID NOS: 28, 30, 32, 34, 36, or 38 or may encode a coronavirus N protein immunogen variant as described herein (which retains at least one biological or functional activity such as, for example, capability to bind to an N protein specific antibody that is a protective antibody, such as a neutralizing antibody; the capability to elicit or induce the production of an antibody that specifically binds to an N protein immunogen; the capability to elicit or induce an immune response (humoral and/or cell-mediated); or the capability to bind to a nucleic acid molecule such as the coronavirus genomic RNA). Reference to one or more isolated nucleic acids includes variants of these sequences that are substantially similar in that they encode native or non-native coronavirus N polypeptides with similar structure and have the capability to elicit specific antibodies to at least one N protein epitope contained in the coronavirus N protein-derived polypeptides of SEQ ID NOS:28, 30, 32, 34, 36, or 38. Thus in certain embodiments, isolated nucleic acids (or polynucleotides) include variants of SEQ ID NOS: 27, 29, 31, 33, 35, and 37 that are substantially similar to these sequences in that the variant nucleotide sequences encode native or non-native coronavirus N polypeptides with similar structure and ability to elicit specific antibodies to at least one N protein epitope contained in the coronavirus N protein polypeptide set forth in SEQ ID NOS:28, 30, 32, 34, 36, or 38.

As used herein, a nucleotide sequence is deemed to be “substantially similar” to a nucleotide sequence that encodes a coronavirus S protein or N protein, variant, or fragment thereof if (a) the nucleotide sequence is derived from the coding region of a coronavirus S or N protein gene (including, for example, nucleotide sequences provided herein and known in the art (such as may be found in GenBank and other sequence databases); sequences derived from different strains of a coronavirus, such as strains of SARS coronavirus; or portions of such sequences) and contains an S or N protein epitope with substantially the same capability to elicit an immune response (humoral or cell-mediated immune response), preferably a protective immune response; (b) a polynucleotide comprising the substantially similar nucleotide sequence is capable of hybridizing to a nucleotide sequence or its complement as described herein that encodes an S or N protein immunogen under moderate or high stringency conditions; and/or (c) the nucleotide sequence is degenerate (i.e., the sequences comprise codon sequences that differ but code for the same amino acid); or (d) the sequence is a complement of any of the sequences described in (a), (b) or (c).

As used herein, two nucleotide sequences are said to “hybridize” under conditions of a specified stringency when stable hybrids are formed between substantially complementary nucleic acid sequences. Stringency of hybridization refers to a description of the environment or conditions under which hybrids are annealed and washed, which typically include ionic strength and temperature. Other factors that might affect hybridization include the probe size and the length of time the hybrids are allowed to form. For example, “high,” “medium,” and “low” stringency encompass the following exemplary conditions or equivalent conditions thereto: high stringency is 0.1×SSPE or SSC, 0.1% SDS, at about 65° C.; medium stringency is 0.2×SSPE or SSC, 0.1% SDS, at about 50° C.; and low stringency is 1.0×SSPE or SSC, 0.1% SDS, at about 42° C. As used herein, the term “high stringency conditions” means that one or more sequences will remain hybridized only if the hybridizing nucleotide sequences share at least 95% or at least 97% identity. Suitable moderately stringent conditions include, for example, pre-washing in a solution of 5×SSC, 0.5% SDS, 1.0 mM EDTA (pH 8.0); hybridizing at 50° C.-70° C., 5×SSC for 1-16 hours; followed by washing once or twice at 22-65° C. for 20-40 minutes with one or more each of 2×, 0.5× and 0.2×SSC containing 0.05-0.1% SDS. For additional stringency, conditions may include a wash in 0.1×SSC and 0.1% SDS at 50-60° C. for 15 minutes.

As known to those having ordinary skill in the art, variations in stringency of hybridization conditions may be achieved by altering the time, temperature, and/or concentration of the solutions used for pre-hybridization, hybridization, and wash steps. In addition, conditions for hybridization can be altered according to methods known in the art, for example, by adding formamide to hybridization solutions and concomitantly decreasing the temperature for hybridization.

In certain embodiments, the nucleic acid sequences that remain hybridized to a coronavirus polypeptide-encoding nucleic acid molecule encode polypeptides that retain at least one epitope of an S protein or fragment as set forth in any one of SEQ ID NOS:2, 4, 6, 8, 10, 12, 14, 16, 18, 20, 22, 24, or 26, or that retain at least one epitope of an N protein or fragment as set forth in any one of SEQ ID NOS:28, 30, 32, 34, 36, or 38, wherein one or more epitopes have substantially the same ability to elicit an immune response (humoral and/or cell-mediated immune response), preferably a protective immune response, as the native or wildtype S or N protein, respectively. An S or N protein encoded by a nucleic acid molecule that remains hybridized to the nucleotide sequence set forth in any one of SEQ ID NOS:1, 3, 5, 7, 9, 11, 13, 15, 17, 19, 21, 23, or 25 or in any one of SEQ ID NOS: 27, 29, 31, 33, 35, and 37, respectively, may also exhibit at least one of any other functional or biological activities of an S or N protein, respectively, described herein.

Proteins described herein may be constructed or produced using a wide variety of techniques as described herein and practiced in the art. Methods for producing the coronavirus polypeptides include expression of the nucleic acid molecules encoding these polypeptides in a host cell. In one embodiment, a method of producing an S or N protein immunogen (having at least one epitope that elicits a protective immune response against coronavirus infection) comprises culturing a host cell containing a nucleic acid expression vector comprising at least one expression control sequence such as a promoter operably linked to a nucleic acid molecule encoding a coronavirus polypeptide, such as a coronavirus polypeptide as set forth in any one of SEQ ID NOS:2, 4, 6, 8, 10, 12, 14, 16, 18, 20, 22, 24, or 26 (or a variant or fragment thereof as described herein), or as set forth in any one of SEQ ID NOS:28, 30, 32, 34, 36, or 38 (or a variant or fragment thereof as described herein), under conditions and for a time sufficient for expression of the S or N immunogen, respectively. These expression vectors or vector constructs that include a polynucleotide sequence encoding the desired protein preferably is operably linked to suitable transcriptional or translational regulatory elements. Selection of appropriate regulatory elements is dependent on the host cell chosen and may be readily accomplished by one of ordinary skill in the art. Examples of regulatory elements include a transcriptional promoter and enhancer or RNA polymerase binding sequence, a transcriptional terminator, and a ribosomal binding sequence including a translation initiation signal. Optionally, the vector may include a polyadenylation sequence, one or more restriction sites, as well as one or more selectable markers such as neomycin phosphotransferase or hygromycin phosphotransferase or any other markers known in the art. Additionally, depending on the host cell chosen and the vector employed, other genetic elements such as an origin of replication, additional nucleic acid restriction sites, enhancers, sequences conferring inducibility of transcription, and selectable markers, may also be incorporated into the vectors described herein.

Bacterial expression vectors preferably comprise a promoter that functions in the host cell, one or more selectable phenotypic markers, and a bacterial origin of replication. In certain embodiments, the nucleic acid expression constructs described herein have an inducible promoter, which may be lac, tac, trc, ara, trp, λ phage, T7 phage, and T5 phage promoter, or may be a T5 phage promoter/lac operator expression control sequence (plasmid pT5) as described in U.S. Patent Application Publication No. 2003/0143685. The expression control sequence refers to any sequence sufficient to allow expression of a protein of interest in a host cell, including one or more promoter sequences, enhancer sequences, operator sequences (e.g., lacO), and the like. In certain embodiments, the coronavirus polypeptide-encoding nucleic acid (such as a nucleic acid encoding an S or N protein immunogen, or a variant thereof) is incorporated into a plasmid, such as plasmid pT5, and the host cell is a bacterium, for example, Escherichia coli.

Other representative promoters include the β-lactamase (penicillinase) and lactose promoter system (see Chang et al., Nature 275:615, 1978), the T7 RNA polymerase promoter (Studier et al., Meth. Enzymol. 185:60-89, 1990), the lambda promoter (Elvin et al., Gene 87:123-126, 1990), the trp promoter (Nichols and Yanofsky, Meth. in Enzymology 101:155, 1983), and the tac promoter (Russell et al., Gene 20:231, 1982). Additional promoters include promoters capable of recognizing the T4, T3, Sp6 and T7 polymerases, the P_{R} and P_{L} promoters of bacteriophage lambda, the recA, heat shock, lacUV5, tac, lpp-lacSpr, phoA, and lacZ promoters of E. coli, promoters of B. subtilis, the promoters of the bacteriophages of Bacillus, Streptomyces promoters, the int promoter of bacteriophage lambda, the bla promoter of pBR322, and the CAT promoter of the chloramphenicol acetyl transferase gene. Prokaryotic promoters have been reviewed by Glick, J. Ind. Microbiol. 1:277, 1987, Watson et al., Molecular Biology of the Gene, 4th Ed. (Benjamin Cummins 1987), and by Ausubel et al. (1995). Representative selectable markers include various antibiotic resistance markers such as the kanamycin or ampicillin resistance genes. Many plasmids suitable for transforming host cells are well known in the art, including among others, pBR322 (see Bolivar et al., Gene 2:95, 1977), the pUC plasmids pUC18, pUC19, pUC118, pUC119 (see Messing, Meth. in Enzymology 101:20-77, 1983 and Vieira and Messing, Gene 19:259-268, 1982), and pNH8A, pNH16a, pNH18a, and Bluescript M13 (Stratagene, La Jolla, Calif.).

In certain embodiments, the S and N protein immunogens or variants thereof are expressed in the same cell, or from the same expression vector, or from the same expression vector as a hybrid fusion polypeptide. Further, mutations may be introduced at particular loci by synthesizing oligonucleotides that contain a mutant sequence that are flanked by restriction sites, enabling ligation to fragments of the native sequence. Following ligation, the resulting reconstructed sequence encodes a derivative or variant having the desired amino acid insertion, substitution, or deletion.

Alternatively, oligonucleotide-directed site-specific (or segment specific) mutagenesis procedures may be employed to provide an altered polynucleotide having particular codons altered according to the substitution, deletion, or insertion. Exemplary methods of making the alterations set forth above are disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and Sambrook et al. (supra). Deletion or truncation derivatives of proteins (e.g., a soluble extracellular portion) may also be constructed by using convenient restriction endonuclease sites adjacent to the desired deletion. Subsequent to restriction, overhangs may be filled in and the DNA religated. Exemplary methods of making the alterations set forth above are disclosed by Sambrook et al. (Molecular Cloning: A Laboratory Manual, 3d Ed., Cold Spring Harbor Laboratory Press (2001)).

Mutations that are made in the nucleic acid molecules preferably preserve the reading frame of the coding sequences. Furthermore, the mutations will preferably not create complementary regions that when transcribed could hybridize to produce secondary mRNA structures, such as loops or hairpins, which would adversely affect translation of the mRNA. Although a mutation site may be predetermined, the nature of the mutation need not per se be predetermined. For example, in order to select for optimum characteristics of mutants at a given site, random mutagenesis may be conducted at the target codon and the expressed mutants screened for gain, loss, or retention of biological activity. Alternatively, mutations may be introduced at particular loci by synthesizing oligonucleotides containing a mutant sequence, flanked by restriction sites enabling ligation to fragments of the native sequence. Following ligation, the resulting reconstructed sequence encodes a derivative having the desired amino acid insertion, substitution, or deletion. Nucleic acid molecules that encode proteins of the present invention may also be constructed using techniques such as polymerase chain reaction (PCR) mutagenesis, chemical mutagenesis (Drinkwater and Klinedinst, Proc. Natl. Acad. Sci. USA 83:3402-3406, 1986); forced nucleotide misincorporation (e.g., Liao and Wise Gene 88:107-111, 1990); or use of randomly mutagenized oligonucleotides (Horwitz et al., Genome 3:112-117, 1989).

Vector constructs comprising cloned polynucleotide sequences encoding any one of the coronavirus proteins described herein can be introduced into cultured mammalian cells by, for example, liposome-mediated transfection, calcium phosphate-mediated transfection (Wigler et al., Cell 14:725, 1978; Corsaro and Pearson, Somatic Cell Genetics 7:603, 1981; Graham and Van der Eb, Virology 52:456, 1973), electroporation (Neumann et al., EMBO J. 1:841-845, 1982), or DEAE-dextran mediated transfection (Ausubel et al. (eds.), Current Protocols in Molecular Biology, John Wiley and Sons, Inc., NY, 1987); retroviral, adenoviral and protoplast fusion-mediated transfection (see Sambrook et al., supra). To identify cells that have been stably transfected with the vector containing the cloned DNA, a selectable marker is generally introduced into the cells along With the polynucleotide of interest. Preferred selectable markers for use in cultured mammalian cells include genes that confer resistance to drugs, such as neomycin, hygromycin, and methotrexate. The selectable marker may be an amplifiable selectable marker. Preferred amplifiable selectable markers are the DHFR gene and the neomycin resistance gene. Selectable markers are reviewed by Thilly (Mammalian Cell Technology, Butterworth Publishers, Stoneham, Mass.).

Multivalent Vaccines

The polynucleotides and host cells described herein may be used to make multivalent immunogens, which may be used for example, as multivalent vaccines, and which may comprise at least one S protein immunogen, or one or more S protein immunogens, that is, a mixture or combination of a plurality of different S protein immunogens. In another embodiment, a multivalent vaccine comprises at least one N protein immunogen, or one or more N protein immunogens, that is, a mixture or combination of a plurality of different N protein immunogens. Alternatively, a multivalent vaccine may comprise a combination of one or more S protein immunogens with one or more coronavirus N protein immunogens and/or other coronavirus immunogens, such as M protein. In another embodiment, the multivalent vaccine is a multivalent hybrid vaccine and comprises at least two or a plurality of the aforementioned immunogens that are linked in some manner, such as for example, fused in frame as a fusion protein. In addition, the immunogen fusion protein may have one or more immunogens reiterated at least once within the fusion protein (such that the at least one immunogen is contained at least at two locations in the fusion protein), which reiteration may occur at the amino- or carboxy-terminal of the selected multivalent immunogen polypeptide, or internal to the multivalent fusion protein. For example, such multivalent hybrid coronavirus immunogens (multivalent fusion proteins) may comprise (1) one or more S protein immunogens or polypeptide fragments of the S protein as described herein (such as for example SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26, and variants and fragments thereof); (2) one or more N protein immunogens or polypeptide fragments of the N protein as described herein (such as, for example, SEQ ID NO:28, SEQ ID NO:30, SEQ ID NO:32, SEQ ID NO:34, SEQ ID NO:36, and SEQ ID NO:38, and variants and fragments thereof); (3) or one or more S protein immunogens (or fragments thereof) and one or more N protein immunogens (or fragments thereof). At least two or more of a coronavirus S protein immunogen or at least two or more of a coronavirus N protein immunogen is considered a plurality of S protein immunogens or N protein immunogens, respectively. In other embodiments, a multivalent hybrid coronavirus immunogen is combined with an adjuvant, such as Proteosome or Protollin, or with adjuvants such as alum, Freund's adjuvant, or Ribi adjuvants (Corixa Corporation, Seattle, Wash.).

Further, such multivalent hybrid coronavirus immunogen vaccine compositions may combine immunogenic epitopes from different coronavirus antigenic groups, for example, group 1 (e.g., transmissible gastroenteritis virus, TGEV; human respiratory coronavirus, HcoV-229E); group 2 (e.g., mouse hepatitis virus, MHV); group 3 viruses (e.g., avian IBV); and SARS group (e.g., SARS-CoV strains Tor2 (see GenBank Accession No. AY274119); Urbani (see GenBank Accession No. AY278741); Frankfurt 1 (see GenBank Accession No. AY291315), TW5 (see GenBank Accession No. AY502928); BJ01 (see GenBank Accession No. AY278488); GD03T0013 (see GenBank Accession No. AY525636); etc.); or a combination thereof (or any other coronavirus group identified that, for example, infects humans).

In certain embodiments, the same or different coronavirus immunogens may be linked by at least two amino acids encoded by a nucleic acid sequence that is a restriction enzyme recognition site, wherein the restriction sites may be any one or more of BamHI, ClaI, EcoRI, HindIII, KpnI, NcoI, NheI, PmlI, PstI, SalI, XhoI, and the like. Additional amino acid linkers may also be added synthetically as described herein. Preferably, the additional amino acids do not create any identity in sequence within a five amino acid stretch of a human protein. In addition, the hybrid coronavirus immunogen polypeptides may further comprise at least one additional carboxy-terminal amino acid, wherein the additional amino acid is a D-amino acid or an L-amino acid. Any of the twenty naturally occurring amino acids or derivatives thereof may be added, such as cysteine, histidine, leucine, and glutamic acid. For example, the addition of at least one cysteine residue at the carboxy terminal end of the fusion polypeptide may be useful for attachment or linkage of other constituents, such as a lipid, a carrier protein, a tag, an enzyme, and the like.

In certain embodiments, a coronavirus S protein immunogen and/or a coronavirus N protein immunogen is linked to a second amino acid sequence; in a certain particular embodiment the S protein immunogen and/or a coronavirus N protein immunogen is fused in frame with a second amino acid sequence. The second amino acid sequence may comprise a carrier protein (for example, proteins and polypeptides understood in the art to facilitate increased or improved immunogenicity of an antigen), a tag (such as a histidine tag), or an enzyme.

As described herein, a coronavirus immunogen fusion protein may comprise an S or N protein immunogen, fragment, or variant thereof fused to an additional functional or non-functional non-coronavirus polypeptide sequence that permits, for example, detection, isolation, or purification of the hybrid polypeptide fusion proteins. For instance, an additional functional polypeptide sequence may be a tag sequence, which in certain embodiments allows that the fusion protein may be detected, isolated, and/or purified by protein-protein affinity (e.g., receptor-ligand), metal affinity, or charge affinity methods. In certain other embodiments, the hybrid polypeptide fusion proteins may be detected by specific protease cleavage of a fusion protein having a sequence that comprises a protease recognition sequence, such that the hybrid coronavirus polypeptide may be separable from the additional polypeptide sequence. In addition, the hybrid polypeptides may be made synthetically including additional amino acids, a carrier protein, a hydrophobic portion (e.g., a lipid), or a tag sequence, which may be located at either the amino- or carboxy-terminal end. In one embodiment, for example, recombinant coronavirus immunogens are fused in-frame to a tag, which tag may be any one of alkaline phosphatase, thioredoxin, β-galactosidase, hexahistidine (6×His), FLAG® epitope tag (DYKDDDDK, SEQ ID NO:40), or GST, and the like.

In certain embodiments the tag that is fused to a hybrid coronavirus polypeptide fusion protein facilitates affinity detection and isolation of the hybrid coronavirus polypeptide fusion protein, and may include, for example, poly-His or the defined antigenic peptide epitopes described in U.S. Pat. No. 5,011,912 and in Hopp et al., (1988 Bio/Technology 6:1204), or the XPRESS™ epitope tag (DLYDDDDK, SEQ ID NO:41; Invitrogen, Carlsbad, Calif.), or thioredoxin. The affinity sequence may be a hexa-histidine tag as supplied by a vector. For example, a pBAD/His (Invitrogen) or a pQE-30 (Qiagen, Valencia, Calif.) vector can provide a polyhistidine tag for purification of the mature protein fusion from a particular host, such as a bacterium, using a nickel affinity column. Alternatively, the affinity sequence may be added either synthetically or engineered into the primers used to recombinantly generate the nucleic acid sequence (e.g., using the polymerase chain reaction) encoding an immunogenic polypeptide of a coronavirus. For example, in one embodiment, coronavirus immunogens are fused to a thioredoxin and the coronavirus immunogen-thioredoxin fusion protein is encoded by a recombinant nucleic acid sequence.

Therapeutic Formulations and Methods of Use

In certain embodiments, pharmaceutical compositions are provided that contain one or more coronavirus immunogens, which may be used to elicit or induce an immune response against coronavirus. Such compositions may be used in methods for treating and/or preventing a coronavirus infection by administering to a subject an S protein immunogen, fragment, or variant thereof, an S immunogen fusion protein or multivalent immunogen, or a mixture of such immunogens at a dose sufficient to elicit antibodies specific for coronavirus, as described herein. In another embodiment, a method for treating and/or preventing a coronavirus infection comprises administering to a subject an N protein immunogen, fragment, or variant thereof, an N immunogen fusion protein or multivalent immunogen, or a mixture of such immunogens at a dose sufficient to elicit antibodies specific for a coronavirus. In still another embodiment, a method for treating and/or preventing a coronavirus infection comprises administering to a subject at least one S protein immunogen and at least one N protein immunogen (or a variant or fragment of an S or N protein immunogen); or a fusion protein or multivalent immunogen that comprises at least one S protein immunogen and at least one N protein immunogen; or a mixture or cocktail of such immunogens.

As described herein, methods are provided for treating and/or preventing a coronavirus infection. In certain embodiments, one or more coronavirus protein antigens (immunogens) are administered to a subject or host that has a coronavirus infection or is at risk for developing a coronavirus infection. Administration of at least one coronavirus protein (e.g., an S protein immunogen and/or an N protein immunogen) preferably induces or stimulates a protective immune response. A protective immune response as described herein may include a humoral response, that is, administration of the coronavirus protein (immunization) to a subject stimulates or elicits the production of antibodies that specifically bind to the coronavirus protein. Stimulation or elicitation of a humoral response preferably includes production of antibodies that are neutralizing antibodies, which neutralize coronavirus infectivity. A humoral response may also include a mucosal immune response, which comprises production of mucosal IgA antibodies that are specific for coronavirus, and may include production of any one of the various immunoglobulin classes, including IgM, IgG, and IgA that can be detected in sera of a subject or host. Administration of at least one coronavirus protein immunogen may also induce a cell-mediated response, which includes stimulation of T cells, production of immunostimulatory molecules such as cytokines produced by immune cells, and clonal expansion of specific T cells in response to the specific coronavirus protein immunogen.

In one embodiment, a composition that is useful as an immunogenic composition for treating and/or preventing a coronavirus infection contains at least one coronavirus antigen (immunogen) as described herein (including multivalent vaccines and multivalent hybrid fusion proteins) capable of eliciting an immune response and Protollin or Proteosome adjuvant (see, e.g., U.S. Pat. Nos. 5,726,292 and 5,985,284, and U.S. Patent Application Publication Nos. 2001/0053368 and 2003/0044425). As is understood in the art, an adjuvant may enhance or improve the immunogenicity of an immunogen (that is, act as an immunostimulant), and many antigens are poorly immunogenic unless combined or admixed or mixed with an adjuvant. A variety of sources can be used as a source of antigen, such as live-attenuated virus, killed virus, split antigen preparations, subunit antigens, recombinant or synthetic viral antigens, and combinations thereof. To maximize the effectiveness of a subunit, recombinant, or synthetic vaccine, the antigens can be combined with a potent immunostimulant or adjuvant. Other exemplary adjuvants include alum (aluminum hydroxide, REHYDRAGEL®); aluminum phosphate; virosomes; liposomes with and without Lipid A; Detox (Ribi/Corixa); MF59; or other oil and water emulsions type adjuvants, such as nanoemulsions (see, e.g., U.S. Pat. No. 5,716,637) or submicron emulsions (see, e.g., U.S. Pat. No. 5,961,970); and Freund's complete and incomplete adjuvant.

A Proteosome-based adjuvant (i.e., Protollin or Proteosome) can be used in vaccine compositions or formulations that may include any one or more of a variety of coronavirus antigen (immunogen) sources as described herein. Proteosomes are comprised of outer membrane proteins (OMP) from Neisseria species typically, but can be derived from other Gram-negative bacteria (see, e.g., Lowell et al., J. Exp. Med. 167:658, 1988; Lowell et al., Science 240:800, 1988; Lynch et al., Biophys. J. 45:104, 1984; U.S. Pat. No. 5,726,292; U.S. Pat. No. 4,707,543). Proteosomes have the capability to auto-assemble into vesicle or vesicle-like OMP clusters of 20-800 nm, and to noncovalently incorporate, coordinate, associate, or otherwise cooperate with protein antigens, particularly antigens that have a hydrophobic moiety. Proteosomes are hydrophobic, safe for human use, and comparable in size to certain viruses. By way of background, and not wishing to be bound by theory, mixing Proteosomes with an antigen such as a protein antigen, provides a composition comprising non-covalent association or coordination between the antigen and Proteosomes, which association or coordination forms when solubilizing detergent is selectively removed or reduced in concentration, for example, by dialysis. Proteosomes may be prepared as described such as in U.S. Patent Application Nos. 2001/0053368 and 2003/0044425.

Any preparation method that results in the outer membrane protein component in vesicular or vesicle-like form, including molten globular-like OMP compositions of one or more OMP, is included within the definition of “Proteosome.” In one embodiment, the Proteosomes are from Neisseria species, and more preferably from Neisseria meningitidis. In certain other embodiments, Proteosomes may be an adjuvant and an antigen delivery composition. In a preferred embodiment, an immunogenic composition comprises one or more coronavirus antigens and an adjuvant, wherein the adjuvant comprises Projuvant or Protollin. As described herein, a coronavirus antigen may be isolated from the virus particles, a cell infected by the coronavirus, or from a recombinant source and/or may comprise, for example, a (detergent) split antigen.

In certain embodiments, an immunogenic composition further comprises a second immunostimulant, such as a liposaccharide. That is, the adjuvant may be prepared to include an additional immunostimulant. For example, the Projuvant may be mixed with a liposaccharide to provide an OMP-LPS adjuvant. Thus, the OMP-LPS (Protollin) adjuvant can be comprised of two components. The first component includes an outer membrane protein preparation of Proteosomes (i.e., Projuvant) prepared from Gram-negative bacteria, such as Neisseria meningitidis, and the second component includes a preparation of liposaccharide. The liposaccharide may be prepared as described in U.S. Patent Application Nos. 2001/0053368 and 2003/0044425. It is also contemplated that the second component may include lipids, glycolipids, glycoproteins, small molecules or the like, and combinations thereof.

As described herein, the two components of an OMP-LPS adjuvant may be combined (admixed or formulated) at specific initial ratios to optimize interaction between the components, resulting in stable association and formulation of the components for use in the preparation of an immunogenic composition. The process generally involves the mixing of components in a selected detergent solution (e.g., Empigen® BB, Triton® X-160, or Mega-10) and then effecting complex formation of the OMP and LPS components while reducing the amount of detergent to a predetermined, preferred concentration by dialysis or by diafiltration/ultrafiltration methodologies. Mixing, co-precipitation, or lyophilization of the two components may also be used to effect an adequate and stable association, composition, or formulation. In one embodiment, an immunogenic composition comprises one or more coronavirus antigens and an adjuvant, wherein the adjuvant comprises a Projuvant (i.e., Proteosome) and liposaccharide.

In a particular embodiment, the final liposaccharide content by weight as a percentage of the total Proteosome protein can be in a range from about 1% to about 500%, more preferably in range from about 10% to about 200%, or in a range from about 30% to about 150%. Another embodiment includes an adjuvant wherein the Proteosomes are prepared from Neisseria meningitidis and the liposaccharide is prepared from Shigella flexneri or Plesiomonas shigelloides, and the final liposaccharide content is between 50% to 150% of the total Proteosome protein by weight. In another embodiment, Proteosomes are prepared with endogenous lipooligosaccharide (LOS) content ranging from about 0.5% up to about 5% of total OMP. In another embodiment Proteosomes have endogenous liposaccharide in a range from about 12% to about 25%, and in still another embodiment the endogenous liposaccharide is between about 15% and about 20% of total OMP. The instant disclosure also provides a composition containing liposaccharide derived from any Gram-negative bacterial species, which may be from the same Gram-negative bacterial species that is the source of Proteosomes or may be from a different bacterial species.

In certain embodiments, the Proteosome or Protollin to coronavirus antigen ratio in the immunogenic composition is greater than 1:1, greater than 2:1, greater than 3:1 or greater than 4:1. In other embodiments, Proteosome or Protollin to coronavirus antigen ratio in the immunogenic composition is about 1:1, 2:1, 3:1, or 4:1. The ratio can be 8:1 or higher. In other embodiments, the ratio of Proteosome or Protollin to coronavirus antigen of the immunogenic composition ranges from about 1:1 to about 1:500, and is at least 1:5, at least 1:10, at least 1:20, at least 1:50, or at least 1:100, or at least 1:200. An advantage of Protollin:coronavirus antigen ratios ranging from 1:2 to 1:200 is that the amount of Proteosome-based adjuvant can be reduced dramatically with no significant effect on the ability of a coronavirus antigen to elicit an immune response.

In another embodiment, a composition comprises one or more coronavirus S protein immunogens combined (admixed or formulated) with Proteosome or Protollin, wherein the S protein immunogen comprises an amino acid sequence that is identical to, or at least 80% identical (which includes at least 85%, 90%, or 95% or any percent in between 80% and 100%) to SEQ ID NO:2 or fragment thereof and wherein the S protein immunogen or fragment thereof has an epitope that elicits a protective immune response against coronavirus infection. An exemplary S protein immunogen comprises an amino acid sequence as set forth in SEQ ID NO:2 or consisting of SEQ ID NO:2. In other embodiments, an S protein immunogen is a fragment of SEQ ID NO:2, which fragment comprises an amino acid sequence that is identical to, or at least 80% identical (which includes at least 85%, 90%, or 95% or any percent in between 80% and 100%) to an amino acid selected from SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26.

In other embodiments, immunogenic compositions are comprised of one or more coronavirus N protein immunogens, fragments, or variants, thereof and an adjuvant, wherein the adjuvant comprises Proteosomes or Protollin. In certain embodiments, the N protein immunogen comprises the amino acid sequence set forth in SEQ ID NO:28, and in certain other embodiments, the composition comprises an N protein immunogen variant that has a sequence at least 80% that is identical to (which includes at least 85%, 90%, or 95% or any percent in between 80% and 100%) the amino acid sequence set forth in SEQ ID NO:28. Exemplary N protein immunogens or variants thereof for use in these immunogenic compositions include amino acid sequences that are fragments of SEQ ID NO:28, and that, for example, comprise an amino acid sequence of SEQ ID NO:30, SEQ ID NO:32, SEQ ID NO:34, SEQ ID NO:36, or SEQ ID NO:38, or sequences at least 80% identical to these sequences (which includes at least 85%, 90%, or 95% or any percent in between 80% and 100%).

In another embodiment, immunogenic compositions are comprised of at least one (one or more) coronavirus S immunogen, fragment, or variant, thereof and at least one (one or more) coronavirus N protein immunogen, fragment, or variant, thereof and an adjuvant, wherein the adjuvant comprises Projuvant or Projuvant and liposaccharide.

Alternatively, any S or N protein immunogen or any combination of S and N protein immunogens as described herein can be combined (admixed or formulated) in an immunogenic composition with a liposome. Preferably, liposomes that contain one or more coronavirus immunogens further comprise Deinococcus radiodurans lipids or α-galactosylphosphotidylglycerolalkylamine. The addition of such lipids in a liposome can enhance the efficacy of a coronavirus vaccine composition by increasing protective immunity.

Coronavirus polypeptides and immunogens of the present invention may further include a covalently attached hydrophobic portion. A hydrophobic portion may be, for example, an amino acid sequence or a lipid, as disclosed in U.S. Pat. No. 5,726,292. Naturally occurring coronavirus S protein and a recombinantly expressed S protein having the sequence set forth in SEQ ID NO:2 contains a hydrophobic transmermbrane domain (from about amino acid 1195 to about 1240 of SEQ ID NO:2), which may function as a hydrophobic portion with an S protein immunogen fragment (e.g., SEQ ID NOS:16 or 18 can have the hydrophobic transmembrane domain from S protein fused thereto) or N protein immunogen (e.g., SEQ ID NOS:30 or 32 can have the hydrophobic transmembrane domain from S protein fused thereto). In one embodiment, a coronavirus composition (e.g., a vaccine composition) comprises a coronavirus S protein or N protein immunogen, or variant thereof, combined, admixed, complexed, or formulated with a Proteosome (see, e.g., U.S. Pat. Nos. 5,726,292 and 5,985,284) or Protollin (see, e.g., U.S. Patent Application No. 2003/0044425), wherein the S or N protein immunogen further comprises a hydrophobic portion or foot. When combined with a Proteosome, the S or N protein immunogens preferably include a hydrophobic portion, which may be composed of a hydrophobic amino acid sequence or a lipid (as used herein, lipid refers to a solubility characteristic and, therefore, includes alkyls, arylalkls, aryls, fatty acids, glycerides and glyceryl ethers, phospholipids, sphingolipids, long chain alcohols, steroids, vitamins, and the like). In one embodiment, the hydrophobic portion of S protein (e.g., the transmembrane domain) can be fused to a coronavirus N protein immunogen. In certain other embodiments, the S or N protein immunogens, with or without a hydrophobic portion, may further contain a second amino acid sequence to form a fusion protein, wherein the second amino acid sequence is a tag, carrier, or enzyme, as described herein. In still other embodiments, the S and N immunogens can be combined in an immunogenic composition, as separate components or fused to form a hybrid, multivalent immunogen, with or without a hydrophobic portion, and further with, or alternatively with, a second amino acid sequence as described herein.

In other embodiments, immunogenic compositions may comprise (Projuvant or Protollin), or further comprise, components (e.g., receptor ligands) capable of stimulating a host immune response by interacting with certain receptors (e.g., Toll-like receptors, TLR) produced by one or more host cells of a vaccine recipient. According to one embodiment, compositions comprising immunogenic epitopes of a coronavirus protein may contain polypeptide epitopes capable of interacting with Toll-like receptors (TLRs), thereby promoting an innate immune response, which may or may not evoke a subsequent adaptive immune response.

An innate immune response is a nonspecific protective immune response that is not a specific antigen-dependent or antibody-dependent response (that is, does not involve clonal expansion of T cells and/or B cells) and may be elicited by any one of numerous antigens, immunogens, or coronaviruses described herein. An immunostimulatory composition described herein comprises Proteosomes and liposaccharide (Protollin), either one of which or both may elicit a nonspecific protective response. Without wishing to be bound by theory, one or more components of vaccine compositions or formulations disclosed herein may interact with Toll-like receptors (TLRs) associated with an innate or adaptive immune response of a vaccine recipient. At least 10 TLRs are described (see, e.g., Takeda et. al., Annu. Rev. Immunol. 21:335, 2003). One or more ligands that interact with and subsequently activate certain TLRs have been identified, with the exception of TLR8 and TLR10. Certain outer membrane proteins of Neisseria meningitidis, for example OMP 2 (also referred to as Por B), interact with TLR2, while LPS of most but not all Gram-negative bacteria interacts with TLR4. Accordingly, one activity of vaccine compositions or formulations described herein, which may contribute to a biological effect, includes activation of one or both of TLR2 and TLR4. Activation of other TLRs (other than TLR2 and TLR4) may serve a similar function or further enhance the qualitative or quantitative profile of cytokines expressed, and may be associated with a host Th1/Th2 immune response. It is also contemplated that TLR ligands other than LPS and Por B may be used alone or in combination to activate TLR2 or TLR4. The qualitative or quantitative activation of one or more TLRs is expected to elicit, effect, or influence a relative stimulation (balanced or imbalanced) of a Th1 (type 1) or Th2 (type 2) immune response, with or without a concomitant humoral antibody response. Ligands interacting with TLRs other than TLR2 and TLR4 may also be used in vaccine compositions described herein. Such vaccine components may, alone or in combination, be used to influence the development of a host immune response sufficient to treat or protect from virus infection, as set forth herein. Such TLRs and associated ligands are known in the art, which include those presented in Table 1.

TABLE 1
TLRs and Ligands

| TLR family | Ligands |
|---|---|
| TLR1 | Soluble factors (e.g., Neisseria meningitidis) |
| | Tri-acyl lipopeptides (bacteria, mycobacteria) |
| TLR2 | Lipoproteins and lipopeptides |
| | Porins (Neisseria) |
| | Atypical LPS (e.g., Leptospira interrogans, P. gingivalis) |
| | Peptidoglycan (Gram-positive bacteria) |
| | Lipoteichoic acid (Gram-positive bacteria) |
| | HSP70 (host) |
| | Glycolipids (e.g., Treponema maltophilum) |
| TLR3 | Double-stranded RNA (e.g., viral) |
| TLR4 | LPS (Gram-negative bacteria) |
| | Taxol (plant) |
| | HSP60 (host) |
| | HSP70 (host) |
| | HSP60 (Chlamydia pneumoniae) |
| | Fibrinogen (host) |
| TLR5 | Flagellin (bacteria) |
| TLR6 | Di-acyl lipopeptides (mycoplasma) |
| TLR7 | Imidazoquinoline (synthetic compounds) |
| | Loxoribine (synthetic compounds) |
| | Bropirimine (synthetic compounds) |
| TLR8 | Ligand yet to be identified |
| TLR9 | CpG DNA (bacteria) |
| TLR10 | Ligand yet to be identified |

Any one or any combination of the identified TLRs (Table 1) may be activated by any one or any combination of TLR ligand components added to, combined with, or formulated in a vaccine composition comprising a coronavirus S protein immunogen, N protein immunogen, or both an at least one S protein immunogen and an N protein immunogen as described herein. The stimulation of any one or a multiplicity of TLRs may be accomplished using any one or a multiplicity of TLR ligands at concentrations suitable with the route of administration (e.g., intranasal, injection, etc.). Therefore, a vaccine composition or formulation may include any one or more TLR ligand(s), including recombinant ligands (fusion proteins or fragments thereof) combined or formulated with an antigenic (immunogenic) vaccine component, with or without addition of an exogenous liposaccharide component.

An efficient immune response depends on the communication between the innate and adaptive immune responses. The T lymphocyte is important for coordinating the adaptive immune response by controlling the release of effector molecules. For example, T helper (Th) 1 cells produce interleukin-2 (IL-2), tumor necrosis factor alpha (TNF-α), and interferon gamma (IFN-γ), which are important for the development of cell-mediated immunity (Mosmann et al., J. Immunol. 136: 2348, 1986; Street and Mosmann, FASEB J. 5: 171, 1991). In contrast, Th2 cells produce IL-4, IL-13, IL-5, IL-9, IL-6 and IL-10. These effector molecules can be readily measured in a biological sample from a subject or host immunized with any of the coronavirus immunogens described herein according to methods routinely practiced by persons skilled in the art.

A cell mediated immune (CMI) response includes determining whether an immune response has shifted from a predominantly Th2 response to a balanced or mixed Th1 and Th2 response (due to a an increase in Th1 response or concomitant increase in Th1 and decrease in Th2 response), or to a predominantly Th1 response. Similarly, a shift from a Th1 response to a balanced or mixed Th1/Th2 response or an increased or predominant Th2 response may be determined. For example, levels of Th1 cytokines, such as IFN-γ, IL-2, and TNF-β, and Type 2 cytokines, such as IL-4, IL-5, IL-6, IL-9, IL-10, and IL-13, may be determined according to methods described herein and practiced in the art, including ELISA, ELISPOT, and flow cytometry (to measure intracellular cytokines). Type 1 responses are predictive of induction of other CMI-associated responses, such as development of cytotxic T cells (CTLs), which are indicative of Th1 immunity. Immune cell proliferation and clonal expansion resulting from an antigen-specific elicitation or stimulation of an immune response may be determined by isolating lymphocytes, such as spleen cells or cells from lymph nodes, stimulating the cells with antigen, and measuring cytokine production, cell proliferation and/or cell viability, such as by incorporation of tritiated thymidine or non-radioactive assays, such as MTT assays and the like.

The immunostimulatory, immunogenic, and/or immunomodulatory compositions described herein may induce specific anti-antigen immune response, including one or more of the following. A specific humoral response may be elicited, induced, or stimulated that results in production of antigen specific antibodies, which may include any class of immunoglobulin, including IgG, IgA, IgM, and/or IgE, and isotypes of the classes. For example, the presence of specific IgM, IgG, and IgA, in serum, nasal wash, lung lavage, and in mucosal secretions (particularly IgA), or other tissues may be determined by any of a variety of immunoassays described herein and known in the art, including but not limited to, ELISA, immunoblot, radioimmunoassay, immunohistochemistry, fluorescence activated cell sorting (FACS), Ochterlony, and the like. For detection of antigen or coronavirus specific antibodies in an immunoassay, the biological sample may be permitted to interact with or contact an antigen that is purified, isolated, partially isolated, or a fragment thereof, or to interact with or contact the virus, which may be fixed (such as with ethanol or formaldehyde) or unfixed or non-denatured. Mucosal secretions include those collected from the respiratory tract, including the nasopharynk and lungs. Functional assays may also be performed, such as the ability of an antigen-specific antibody to facilitate phagocytosis or opsonization of a microorganism, or to prevent entry of a microorganism into a host cell, or to prevent entry, fusion, or propagation of a microorganism such as a virus in a host cell. Such methods are described herein and are routinely practiced by skilled artisans.

The pharmaceutical composition will preferably include at least one of a pharmaceutically acceptable vehicle, carrier, diluent, or excipient, in addition to at least one (one or more) coronavirus immunogen or fusion protein thereof and, optionally, other components. For example, pharmaceutically acceptable carriers suitable for use with a composition of S protein immunogens or fusion proteins thereof, or cocktail of two or more S protein immunogens or fusion proteins thereof, or cocktail of S, N, and/or M immunogens or fusion proteins thereof. Pharmaceutically acceptable carriers for therapeutic use are well known in the pharmaceutical art, for example, see Remington's Pharmaceutical Sciences, Mack Publishing Co. (A. R. Gennaro, ed., 18^{th} Edition, 1990) and in CRC Handbook of Food, Drug, and Cosmetic Excipients, CRC Press LLC (S. C. Smolinski, ed., 1992). The compositions may also include a thickening agent, a buffering agent, a solvent, a humectant, a preservative, a chelating agent, an adjuvant, and the like, and combinations thereof.

A pharmaceutically acceptable salt refers to salts of compounds derived from the combination of such compounds and an organic or inorganic acid (acid addition salts) or an organic or inorganic base (base addition salts). Compounds may be used in either the free base or salt forms.

In addition, the pharmaceutical composition may further include a diluent such as water or phosphate buffered saline (PBS). In certain embodiments the diluent is PBS formulated to deliver into the host a final phosphate concentration and a final sodium chloride concentration that is physiological. PBS may have a final phosphate concentration range from about 0.1 mM to about 50 mM, more preferably from about 0.5 mM to about 40 mM, even more preferably from about 1 mM to about 25 mM, and most preferably from about 2.5 mM to about 10 mM; the final salt concentration ranges from about 100 mM to about 200 mM and most preferably from about 125 mM to about 175 mM. Preferably, the final PBS concentration is about 5 mM phosphate and about 150 mM salt (such as NaCl). In certain embodiments, any of the aforementioned pharmaceutical compositions comprise a cocktail of coronavirus immunogens as described herein, and which are preferably sterile.

A composition described herein can be made sterile by either preparing the composition under an aseptic environment and/or by terminally sterilizing the composition using methods available in the art. Many pharmaceuticals are manufactured to be sterile and this criterion is defined by the. USP XXII <1211>. Sterilization in this embodiment may be accomplished by a number of means accepted in the industry and listed in the USP XXII <1211>, including gas sterilization, ionizing radiation or filtration. Sterilization may be maintained by what is termed aseptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. When appropriate, filtration may be accomplished using a filter with suitable pore size, for example 0.22 μm and of a suitable material, for instance Teflon®. The term “USP” refers to U.S. Pharmacopeia (Rockville, Md.).

Also described herein are methods for treating and/or preventing a coronavirus infection, comprising administering to a subject in need thereof a composition comprising at least one coronavirus S protein immunogen, wherein the S protein immunogen comprises an amino acid sequence that is identical to, or at least 80% identical to (which includes at least 85%, 90%, or 95% or any percent in between 80% and 100%) SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26, and wherein the S protein immunogen has an epitope that elicits a protective immune response, which is a humoral immune response (including, for example, a mucosal IgA, systemic IgA, IgG, IgM response) and/or a cell-mediated immune response, and pharmaceutically acceptable carrier, diluent, or excipient. The S protein immunogen composition is administered at a dose sufficient to elicit an immune response specific for the administered S protein immunogen or immunogens or variants thereof. In certain embodiments, an infection being prevented or treated may be caused by a group 1 coronavirus, group 2 coronavirus, group 3 coronavirus, SARS group coronavirus, or a combination thereof.

In other embodiments, a method for treating and/or preventing a coronavirus infection, comprises administering to a subject in need thereof a composition comprising at least one coronavirus N protein immunogen, wherein the N protein immunogen comprises an amino acid sequence that is identical to, or at least 80% identical to (which includes at least 85%, 90%, or 95% or any percent in between 80% and 100%) SEQ ID NO:28, SEQ ID NO:30, SEQ ID NO:32, SEQ ID NO:34, SEQ ID NO:36, or SEQ ID NO:38, and wherein the N protein immunogen has an epitope that elicits a protective immune response (humoral response (including, for example, a mucosal IgA, systemic IgA, IgG, IgM response) and/or a cell-mediated immune response), and a pharmaceutically acceptable carrier, diluent or excipient. The N protein immunogen composition is administered at a dose sufficient to elicit an immune response specific for the administered N protein immunogens or variants thereof. In certain embodiments, the infection being prevented or treated may be caused by a group 1 coronavirus, group 2 coronavirus, group 3 coronavirus, SARS group coronavirus, or a combination thereof.

In still other embodiments, a method for treating and/or preventing coronavirus infection, comprises administering to a subject in need thereof a composition comprising a plurality of coronavirus immunogens. The plurality of coronavirus immunogens may comprise at least two S protein immunogens wherein each of the at least two S protein immunogens comprises an amino acid sequence that is identical to, or at least 80% identical to (which includes at least 85%, 90%, or 95% or any percent in between 80% and 100%) and selected from SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26. In another embodiment, the plurality of coronavirus immunogens may comprise at least two N protein immunogens wherein each of the at least two N protein immunogens comprises an amino acid sequence that is identical to, or at least 80% identical to (which includes at least 85%, 90%, or 95% or any percent in between 80% and 100%) and selected from SEQ ID NO:28, SEQ ID NO:30, SEQ ID NO:32, SEQ ID NO:34, SEQ ID NO:36, or SEQ ID NO:38. In another embodiment, a method for treating and/or preventing a coronavirus infection comprises a plurality of coronavirus protein immunogens that comprises at least one S protein immunogen as described herein and at least one N protein immunogen as described herein. In other embodiments, a method for treating and/or preventing a coronavirus infection may comprise a plurality of coronavirus protein immunogens that may be selected from an S protein immunogen, an N protein immunogen, a coronavirus M protein immunogen, a coronavirus E protein immunogen, and includes any combination thereof. Preferably, each immunogen of the plurality of immunogens has an epitope capable of eliciting a protective immune response such a humoral response (for example, eliciting a neutralizing antibody) and/or a cell-mediated immune response, and is combined with a pharmaceutically acceptable carrier, diluent or excipient. A SARS coronavirus M protein immunogen may have an amino acid sequences such as provided in GenBank Accession No. AAU07933, which is encoded by the nucleotide sequence set forth in GenBank Accession No. AY702026. The nucleotide sequence encoding an M protein and the amino acid sequence of the encoded protein may be found in numerous entries in publicly available databases that provide the nucleotide sequences and encoded amino acid sequences of the entire SARS coronavirus genome. Additional amino acid sequences of coronavirus S protein and coronavirus N protein and the nucleotide sequences encoding these proteins may similarly be found in the SARS coronavirus genome sequences provided in the publicly available databases. In certain embodiments, the immunogen compositions may be specific for group 1 coronavirus, group 2 coronavirus, group 3 coronavirus, SARS group coronavirus, or a combination thereof.

A subject or host suitable for treatment with a- coronavirus immunogen composition or formulation may be identified by well-established indicators of risk for developing a disease or by well-established hallmarks of an existing disease. For example, indicators of an infection include fever, dry cough, dyspnea (shortness of breath), headache, hypoxaemia (low blood oxygen concentration), lymphopaenia (reduced lymphocyte numbers), mildly elevated aminotransferase levels (indicating liver damage), microorganism positive cultures, inflammation, and the like. Infections that may be treated or prevented with a coronavirus immunogen vaccine as described herein include those caused by or due to coronavirus, whether the infection is primary, secondary, or opportunistic. Examples of coronavirus include any subtype, strain, antigenic variant, and the like, of these viruses, including SARS coronavirus. By way of example, SARS infections are characterized by flu-like symptoms, including high fever, myalgia, dry and non-productive dyspnea, lymphopenia, and infiltrate on chest radiography. The mortality rate during the SARS epidemic of 2002-2003 was approximately 10%, but as high as 50% in the elderly (Stadler et al., Nat. Rev. 1:209, 2003).

The pharmaceutical compositions that contain one or more coronavirus immunogens of the invention may be in any form that allows for the composition to be administered to a subject, such as a human or non-human animal. For example, an S or N protein immunogen, fusion protein, and/or multivalent composition may be prepared and administered as a liquid solution or prepared as a solid form (e.g., lyophilized), which may be administered in solid form, or resuspended in a solution in conjunction with administration. The hybrid polypeptide composition is prepared or formulated to allow the active ingredients contained therein to be bioavailable upon administration of the composition to a subject or patient or to be bioavailable via slow release. Compositions that will be administered to a subject or patient take the form of one or more dosage units; for example, a tablet may be a single dosage unit, and a container of one or more compounds of the invention in aerosol form may hold a plurality of dosage units. In certain preferred embodiments, any of the aforementioned pharmaceutical (therapeutic) compositions comprising a coronavirus immunogen or cocktail of immunogens of the invention are in a container, preferably in a sterile container.

In one embodiment, the therapeutic (pharmaceutical) composition is administered nasally, wherein a coronavirus immunogen or cocktail composition can be taken up by cells, such as cells located in the nasal-associated lymphoid tissue. Other typical routes of administration include, without limitation, enteral, parenteral, transdermal/transmucosal, nasal, and inhalation. The term “enteral,” as used herein, is a route of administration in which the immunogenic composition is absorbed through the gastrointestinal tract or oral mucosa, including oral, rectal, and sublingual. The term “parenteral”, as used herein, describes administration routes that bypass the gastrointestinal tract, including intraarterial, intradermal, intramuscular, intranasal, intraocular, intraperitoneal, intravenous, subcutaneous, submucosal and intravaginal injection or infusion techniques. The term “transdermal/transmucosal,” as used herein, is a route of administration in which the immunogenic composition is administered through or by way of the skin, including topical. The terms “nasal” and “inhalation” encompass techniques of administration in which an immunogenic composition is introduced into the pulmonary tree, including intrapulmonary or transpulmonary. In one embodiment, the compositions of the present invention are administered nasally.

In another embodiment, methods are provided for treating and/or preventing a coronavirus infection by administering an antibody that specifically binds to a coronavirus antigen and that facilitates neutralization of the virus (i.e., decreases or eliminates viral infectivity), facilitates inactivation, prevents or inhibits viral assembly, and/or prevents or inhibits viral nucleic acid replication, transcription, or translation. Antibodies that specifically bind to a coronavirus antigen may be generated and prepared by any one of numerous methods described herein and practiced in the art.

In one embodiment, a plurality (at least two or more) of isolated antibodies that specifically bind to a coronavirus protein are produced by a method, which is a method for preventing a coronavirus infection, that comprises administering to a subject a composition containing at least one coronavirus protein immunogen (such as an S protein immunogen, an N protein immunogen, and/or an M protein immunogen) at a dose sufficient to elicit antibodies specific for the at least one coronavirus protein immunogen wherein the protein immunogen has an epitope that elicits a protective immune response, which preferably includes a humoral response. A biological sample, such as serum, lymph, nasopharyngeal washings, blood, ascites, pulmonary washings, or other fluid, may be obtained from the host and the antibodies specific for the coronavirus protein isolated according to methods routinely practiced by a skilled artisan such as affinity purification methods. For example, antibodies that are specific for a coronavirus protein may be removed or isolated from other antibodies and components of the biological sample by contacting the biological sample with a source of the coronavirus protein or fragment thereof. In another embodiment, sera may be obtained from a host immunized with at least one coronavirus protein immunogen and enriched for a particular immnunoglobulin class, such as IgA or IgG. Methods for preparation of such immune sera are well known in the art. The immune sera are preferably isolated from the same host species as the species to which the sera are administered. In a certain embodiment, the antibodies may be obtained from a subject who was immunized with at least one of a group 1 coronavirus, or a group 2 coronavirus, or a group 3 coronavirus, or a SARS group coronavirus, or combination thereof such that the antibodies isolated or the sera obtained from the host comprise at least one antibody specific for a group 1 coronavirus, or a group 2 coronavirus, or a group 3 coronavirus, or a SARS group coronavirus. The biological sample may also contain one or more antibodies that specifically bind to an antigen from more than one group of coronaviruses.

In another embodiment, a method for treating or preventing a coronavirus infection comprises administering to a subject a composition comprising a pharmaceutically acceptable carrier and a plurality of antibodies as described herein. In addition, a subject at risk for acquiring or developing an coronavirus infection can have a plurality of antibodies that specifically bind to a first coronavirus protein immunogen administered before, simultaneous with, or after administration of a composition comprsing at least one coronavirus protein immunogen (for example, a second coronavirus S protein immunogen or a second coronavirus N protein immunogen or a second coronavirus S protein immunogen and a second coronavirus N protein immunogen) that is different from the coronavirus protein immunogen (a first coronavirus S protein immunogen or a first coronavirus N protein immunogen).

As described herein a coronavirus S protein immunogen, or variant thereof, which may be a first or second immunogen or third S protein immunogen etc., comprises an amino acid sequence that is identical to, or at least 80% identical to (which includes at least 85%, 90%, or 95% or any percent in between 80% and 100%), which may be selected from SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26. Also as described herein a coronavirus N protein immunogen, which may be a first or second immunogen or third N protein immunogen etc., comprises an amino acid sequence that is identical to, or at least 80% identical to (which includes at least 85%, 90%, or 95% or any percent in between 80% and 100%) to an amino acid sequence selected from SEQ ID NO:28, SEQ ID NO:30, SEQ ID NO:32, SEQ ID NO:34, SEQ ID NO:36, or SEQ ID NO:38.

In other embodiments, a coronavirus M protein immunogen or a coronavirus E protein immunogen, or combinations thereof, may be administered to elicit an immune response wherein each of the different immunogens, including an S protein immunogen or an N protein immunogen, have at least one epitope that elicits a protective immune response, such as a humoral response or cell-mediated immune response. Such compositions may further comprise a pharmaceutically acceptable carrier, diluent or excipient, as described herein. Thus as described herein, antibodies specific for one or more coronavirus immunogens can be provided passively, while the subject is vaccinated to actively elicit antibodies specific for one or more different coronavirus immunogens. In another embodiment, antibodies specific for one or more coronavirus immunogens can be provided passively, while the subject is vaccinated with one or more of the same as well as one or more different coronavirus immunogens to actively elicit antibodies that specifically bind to one or more coronavirus antigens.

In another embodiment, antibodies are provided that specifically bind to the coronavirus protein immunogens and variants thereof described herein. The coronavirus protein antigens (immunogens), such as an S protein immunogen and an N protein immunogen, or a variant, and fragments of these immunogens, are used to elicit antibodies specific for at least one epitope present on the S or N protein immunogens and variants thereof. In preferred embodiments the antibodies bind to specific protective epitopes present on a coronavirus S or N protein. Antibodies include polyclonal antibodies, monospecific antibodies, monoclonal antibodies, anti-idiotypic antibodies, and antigen-binding fragments thereof such as F(ab′)₂, Fab′, Fd, Fv, and Fab fragments, and recombinantly or synthetically produced antibodies or antigen-binding fragments. Such antibodies incorporate the variable regions that permit a monoclonal antibody to specifically bind, which means an antibody is able to selectively bind to a coronavirus S or N peptide or polypeptide from group 1, group 2, group 3, or SARS group coronaviruses. “Specific for,” “immunospecific,” or “specifically binds” refer to the capability of a protein (e.g., an antibody) to specifically (selectively) bind a polypeptide or peptide encoded by a nucleic acid molecule encoding an immunogen from a coronavirus S or N protein from group 1, 2, 3, or SARS coronaviruses, or a synthesized coronavirus S or N protein from group 1, 2, 3, or SARS coronaviruses. In still another embodiment, a rodent monoclonal antibody (prepared according to methods described herein and known in the art) that specifically binds to a coronavirus protein may be humanized or made fully human according to procedures described herein and known in the art.

“Association” or “binding” of an antibody to a specific antigen generally involves electrostatic interactions, hydrogen bonding, Van der Waals interactions, and hydrophobic interactions. Any one of these or any combination thereof can play a role in the binding between an antibody and its antigen. Such an antibody generally associates with an antigen with an affinity constant (Kₐ) of at least 10⁴, at least 10⁵, at least 10⁶, at least 10⁷, or at least 10⁸. Affinity constants may be determined by one of ordinary skill in the art using well-known techniques (see Scatchard, Ann. N.Y. Acad. Sci. 51:660-672, 1949) and by surface plasmon resonance (SPR; BIAcore™, Biosensor, Piscataway, N.J.; see, e.g., Wolff et al., Cancer Res. 53:2560-2565 (1993)). In addition, binding properties of an antibody to a coronavirus protein immunogen may generally be determined and assessed using immunodetection methods including, for example, an enzyme-linked immunosorbent assay (ELISA), immunoprecipitation, immunoblotting, countercurrent immunoelectrophoresis, radioimmunoassays, dot blot assays, inhibition or competition assays, and the like, which may be readily performed by those having ordinary skill in the art (see, e.g., U.S. Pat. Nos. 4,376,110 and 4,486,530; Harlow et al., Antibodies: A Laboratory Manual, Cold Spring Harbor Laboratory (1988)).

The term “antibody,” as used herein, includes naturally occurring antibodies as well as non-naturally occurring antibodies, including, for example, single chain antibodies, chimeric, bifunctional, and humanized antibodies, as well as antigen-binding fragments thereof. Such non-naturally occurring antibodies may be constructed using solid phase peptide synthesis, may be produced recombinantly, or may be obtained, for example, by screening combinatorial libraries consisting of variable heavy chains and variable light chains (Huse et al., Science 246:1275-1281 (1989)). These and other methods of making, for example, chimeric, humanized, CDR-grafted, single chain, and bifunctional antibodies are well known in the art (see, e.g., Winter and Harris, Immunol. Today 14:243, 1993; Ward et al., Nature 341:544, 1989; Harlow and Lane, Antibodies: A Laboratory Manual, Cold Spring Harbor Laboratory, New York, 1992; Borrabeck, Antibody Engineering, 2d ed., Oxford Univ. Press, 1995; Hilyard et al., Protein Engineering: A practical approach, IRL Press, 1992).

Polyclonal antibodies can be readily generated by one of ordinary skill in the art from a variety of warm-blooded animals, including horses, cows, goats, sheep, dogs, chickens, turkeys, rabbits, mice, hamsters, or rats. Briefly, the desired S protein immunogen or variant thereof or N protein immunogen or variant thereof, or mixtures of coronavirus immunogens or variants thereof, are administered to immunize an animal through parenteral, intraperitoneal, intramuscular, intraocular, or subcutaneous injections, or nasally. The immunogenicity of the polypeptide of interest may be increased through the use of an adjuvant, such as Proteosome, Protollin, alum, Ribi adjuvant, and Freund's complete or incomplete adjuvant. Following several booster immunizations over a period of weeks, small samples of serum are collected and tested for reactivity to the desired immunogen. Once the titer of specific antibodies in the sera of the animal has reached a plateau with regard to reactivity to an S or N protein immunogen or variant thereof, larger quantities of polyclonal immune sera may be readily obtained by periodic, such as weekly bleedings, or by exsanguinating the animal. Polyclonal antibodies may then be purified from such antisera, for example, by affinity chromatography using protein A or protein G immobilized on a suitable solid support (see, e.g., Coligan, supra, p. 2.7.1-2.7.12; 2.9.1-2.9.3; Baines et al., Purification of Immunoglobulin G (IgG), in Methods in Molecular Biology, 10:9-104 (The Humana Press, Inc. (1992)). Alternatively, affinity chromatography may be performed wherein a coronavirus protein antigen (immunogen) to which the antisera specifically bind, or an antibody specific for an Ig constant region of the particular immunized animal species, is immobilized on a suitable solid support.

Monoclonal antibodies that specifically bind to a coronavirus protein antigen and hybridomas, which are examples of immortal eukaryotic cell lines, that produce monoclonal antibodies having the desired binding specificity, may also be prepared, for example, using the technique of Kohler and Milstein (Nature, 256:495-497; 1976, Eur. J. Immunol. 6:511-519 (1975)) and improvements thereto (see, e.g., Coligan et al. (eds.), Current Protocols in Immunology, 1:2.5.1-2.6.7 (John Wiley & Sons 1991); U.S. Pat. Nos. 4,902,614, 4,543,439, and 4,411,993; Monoclonal Antibodies, Hybridomas: A New Dimension in Biological Analyses, Plenum Press, Kennett et al. (eds.) (1980); and Antibodies: A Laboratory Manual, Harlow and Lane (eds.), Cold Spring Harbor Laboratory Press (1988); see also, e.g., Brand et al., Planta Med. 70:986-92 (2004)). An animal—for example, a rat, hamster, or preferably mouse—is immunized with an immunogen prepared as described above. The presence of specific antibody production may be monitored after the initial injection (injections may be administered by any one of several routes as described herein and known in the art for generation of polyclonal antibodies) and/or after a booster injection by obtaining a serum sample and detecting the presence of an antibody that binds to the coronavirus immunogen using any one of several immunodetection methods known in the art and described herein. From animals producing antibodies that bind to the immunogen, lymphoid cells, most commonly cells from the spleen or lymph node, are removed to obtain B-lymphocytes, lymphoid cells that are antibody-forming cells, and then may be immortalized by fusion with a drug-sensitized myeloma (e.g., plasmacytoma) cell fusion partner (e.g., inability to express endogenous Ig gene products, e.g., P3X63-Ag 8.653 (ATCC No. CRL 1580); NS0, SP20). The resulting hybridoma cells may be cultured, isolated, and analyzed according to methods well known in the monoclonal antibody art. The hybridomas are cloned (e.g., by limited dilution cloning or by soft agar plaque isolation) and positive clones that produce an antibody specific to the antigen are selected and cultured. Hybridomas producing monoclonal antibodies with high affinity and specificity for the coronavirus immunogen are preferred.

Monoclonal antibodies may be isolated from the supernatants of hybridoma cultures. An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Contaminants may be removed by conventional techniques, such as chromatography (e.g., size-exclusion, ion-exchange), gel filtration, precipitation, extraction, or the like (see, e.g., Coligan, supra, p. 2.7.1-2.7.12; 2.9.1-2.9.3; Baines et al., Purification of Immunoglobulin G (IgG), in Methods in Molecular Biology, 10:9-104 (The Humana Press, Inc. (1992)). For example, antibodies may be purified by affinity chromatography using an appropriate ligand selected based on particular properties of the monoclonal antibody (e.g., heavy or light chain isotype, binding specificity, etc.). Examples of a suitable ligand, immobilized on a solid support, include Protein A, Protein G, an anti-constant region (light chain or heavy chain) antibody, an anti-idiotype antibody, the specific coronavirus immunogen, or a derivative thereof.

An anti-coronavirus protein antibody may be a human monoclonal antibody. Human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein. Methods for obtaining human antibodies from transgenic mice are described, for example, by Green et al., Nature Genet. 7:13 (1994); Lonberg et al., Nature 368:856 (1994); Taylor et al., Int. Immun. 6:579 (1994); U.S. Pat. No. 5,877,397; Bruggemann et al., Curr. Opin. Biotechnol. 8:455-58 (1997); Jakobovits et al., Ann. N.Y. Acad. Sci. 764:525-35 (1995). Human monoclonal antibodies may be obtained by immunizing the transgenic mice, which may then produce human antibodies specific for the antigen. Lymphoid cells of the immunized transgenic mice can be used to produce human antibody-secreting hybridomas according to the methods described herein. Polyclonal sera containing human antibodies may also be obtained from the blood of the immunized animals.

Another method for generating human coronavirus protein specific monoclonal antibodies includes immortalizing human peripheral blood cells by EBV transformation. See, e.g., U.S. Pat. No. 4,464,456. The stability of the lymphoblastoid cell line producing an anti-coronavirus protein antibody may be improved by fusing the transformed cell line with a murine myeloma to produce a mouse-human hybrid cell line according to methods known in the art (see, e.g., Glasky et al., Hybridoma 8:377-89 (1989)). In certain embodiments, a B cell that is producing an anti-coronavirus protein antibody is selected, and the light chain and heavy chain variable regions are cloned from the B cell according to molecular biology techniques known in the art (WO 92/02551; U.S. Pat. No. 5,627,052; Babcook et al., Proc. Natl. Acad. Sci. USA 93:7843-48 (1996)).

Chimeric antibodies, specific for a coronavirus protein, including humanized antibodies, may also be prepared. A chimeric antibody has at least one constant region domain derived from a first mammalian species and at least one variable region domain derived from a second, distinct mammalian species. See, e.g., Morrison et al., Proc. Natl. Acad. Sci. USA, 81:6851-55 (1984). In one embodiment, a chimeric antibody may be constructed by cloning the polynucleotide sequence that encodes at least one variable region domain derived from a non-human monoclonal antibody, such as the variable region derived from a murine, rat, or hamster monoclonal antibody, into a vector containing a nucleic acid sequence that encodes at least one human constant region (see, e.g., Shin et al., Methods Enzymol. 178:459-76 (1989); Walls et al., Nucleic Acids Res. 21:2921-29 (1993)).

A non-human/human chimeric antibody may be further genetically engineered to create a “humanized” antibody. Such a humanized antibody may comprise a plurality of CDRs derived from an immunoglobulin of a non-human mammalian species, at least one human variable framework region, and at least one human immunoglobulin constant region. Humanization may in certain embodiments provide an antibody that has decreased binding affinity for the specific coronavirus protein when compared, for example, with either a non-human monoclonal antibody from which a coronavirus protein binding variable region is obtained, or a chimeric antibody having such a V region and at least one human C region, as described above. Useful strategies for designing humanized antibodies may therefore include, for example by way of illustration and not limitation, identification of human variable framework regions that are most homologous to the non-human framework regions of the chimeric antibody. Without wishing to be bound by theory, such a strategy may increase the likelihood that the humanized antibody will retain specific binding affinity for the coronavirus protein, which in some preferred embodiments may be substantially the same affinity for the coronavirus protein, and in certain other embodiments may be a greater affinity for the coronavirus protein (see, e.g., Jones et al., Nature 321:522-25 (1986); Riechmann et al., Nature 332:323-27 (1988)).

Designing such a humanized antibody may therefore include determining CDR loop conformations and structural determinants of the non-human variable regions, for example, by computer modeling, and then comparing the CDR loops and determinants to known human CDR loop structures and determinants (see, e.g., Padlan et al., FASEB 9:133-39 (1995); Chothia et al., Nature, 342:377-383 (1989)). Computer modeling may also be used to compare human structural templates selected by sequence homology with the non-human variable regions (see, e.g., Bajorath et al., Ther. Immunol. 2:95-103 (1995); EP-0578515-A3). If humanization of the non-human CDRs results in a decrease in binding affinity, computer modeling may aid in identifying specific amino acid residues that could be changed by site-directed or other mutagenesis techniques to partially, completely or supra-optimally (i.e., increase to a level greater than that of the non-humanized antibody) restore affinity. Those having ordinary skill in the art are familiar with these techniques and will readily appreciate numerous variations and modifications to such design strategies.

Another method for preparing a humanized antibody is called veneering. Veneering framework (FR) residues refers to the selective replacement of FR residues from, e.g., a rodent heavy or light chain V region, with human FR residues in order to provide a xenogeneic molecule comprising an antigen-binding site that retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface (see, e.g., Davies et al., Ann. Rev. Biochem. 59:439-73, (1990)). Thus, antigen binding specificity can be preserved in a humanized antibody when the CDR structures, their interaction with each other, and their interaction with the rest of the V region domains are carefully maintained. By using veneering techniques, exterior (e.g., solvent-accessible) FR residues that are readily encountered by the immune system are selectively replaced with human residues to provide a hybrid molecule that comprises either a weakly immunogenic, or substantially non-immunogenic veneered surface. The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein).

For particular uses, antigen-binding fragments of antibodies that specifically bind to coronavirus proteins may be desired. Antibody fragments, F(ab′)₂, Fab, Fab′, Fv, and Fd, can be obtained, for example, by proteolytic hydrolysis of the antibody, such as by pepsin or papain digestion of whole antibodies according to conventional methods. As an illustration, antibody fragments can be produced by enzymatic cleavage of antibodies with pepsin to provide a fragment denoted F(ab′)₂. This fragment can be further cleaved using a thiol reducing agent to produce an Fab′ monovalent fragment. Optionally, the cleavage reaction can be performed using a blocking group for the sulfhydryl groups that result from cleavage of disulfide linkages. As an alternative, an enzymatic cleavage of an antibody using papain produces two monovalent Fab fragments and an Fc fragment (see, e.g., U.S. Pat. No. 4,331,647; Nisonoffet al., Arch. Biochem. Biophys. 89:230, 1960; Porter, Biochem. J. 73:119, 1959; Edelman et al., in Methods in Enzymology 1:422 (Academic Press 1967); Weir, Handbook of Experimental Immunology, Blackwell Scientific, Boston (1986)).

An antibody fragment may also be any synthetic or genetically engineered protein that acts like an antibody in that it binds to a specific antigen to form a complex. For example, antibody fragments include isolated fragments consisting of the light chain variable region, Fv fragments consisting of the variable regions of the heavy and light chains, recombinant single chain polypeptide molecules in which light and heavy variable regions are connected by a peptide linker (scFv proteins), and minimal recognition units consisting of the amino acid residues that mimic the hypervariable region. The antibodies described herein preferably comprise at least one variable region domain.

A minimal recognition unit is an antibody fragment comprising for a single complementarity-determining region (CDR). Such CDR peptides can be obtained by constructing polynucleotides that encode the CDR of an antibody of interest. The polynucleotides are prepared, for example, by using the polymerase chain reaction to synthesize the variable region using mRNA of antibody-producing cells as a template according to methods practiced by persons skilled in the art (see, for example, Larrick et al., Methods: A Companion to Methods in Enzymology 2:106, (1991); Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley-Liss, Inc. 1995)). Alternatively, such CDR peptides and other antibody fragment can be synthesized using an automated peptide synthesizer.

According to certain embodiments, non-human, human, or humanized heavy chain and light chain variable regions of any of the immunoglobulin molecules described herein may be constructed as scFv polypeptide fragments (single chain antibodies). See, e.g., Bird et al., Science 242:423-426 (1988); Huston et al., Proc. Natl. Acad. Sci. USA 85:5879-5883 (1988)). Multi-functional scFv fusion proteins may be generated by linking a polynucleotide sequence encoding an scFv polypeptide in-frame with at least one polynucleotide sequence encoding any of a variety of known effector proteins. These methods are known in the art, and are disclosed, for example, in EP-B1-0318554, U.S. Pat. No. 5,132,405, U.S. Pat. No. 5,091,513, and U.S. Pat. No. 5,476,786. By way of example, effector proteins may include immunoglobulin constant region sequences. See, e.g., Hollenbaugh et al., 1995 J. Immunol. Methods 188:1-7. Other examples of effector proteins are enzymes. As a non-limiting example, such an enzyme may provide a biological activity for therapeutic purposes (see, e.g., Siemers et al., Bioconjug. Chem. 8:510-19 (1997)), or may provide a detectable activity, such as horseradish peroxidase-catalyzed conversion of any of a number of well-known substrates into a detectable product, for diagnostic uses.

Antibodies may also be identified and isolated from human immunoglobulin phage libraries, from rabbit immunoglobulin phage libraries, and/or from chicken immunoglobulin phage libraries (see, e.g., Winter et al., 1994 Annu. Rev. Immunol. 12:433-55; Burton et al., Adv. Immunol. 57:191-280 (1994); U.S. Pat. No. 5,223,409; Huse et al., Science 246:1275-81 (1989); Schlebusch et al., Hybridoma 16:47-52 (1997) and references cited therein; Rader et al;, J. Biol. Chem. 275:13668-76 (2000); Popkov et al., J. Mol. Biol. 325:325-35 (2003); Andris-Widhopf et al., J. Immunol. Methods 242:159-31 (2000)). Antibodies isolated from non-human species or non-human immunoglobulin libraries may be genetically engineered according to methods described herein and known in the art to “humanize” the antibody or fragment thereof. Immunoglobulin variable region gene combinatorial libraries may be created in phage vectors that can be screened to select Ig fragments (Fab, Fv, scFv, or multimers thereof) that bind specifically to a coronavirus protein (see, e.g., U.S. Pat. No. 5,223,409; Huse et al., Science 246:1275-81 (1989); Sastry et al., Proc. Natl. Acad. Sci. USA 86:5728-32 (1989); Alting-Mees et al., Strategies in Molecular Biology 3:1-9 (1990); Kang et al., Proc. Natl. Acad. Sci. USA 88:4363-66 (1991); Hoogenboom et al., J. Molec. Biol. 227:381-388 (1992); Schlebusch et al., Hybridoma 16:47-52 (1997) and references cited therein; U.S. Pat. No. 6,703,015).

According to certain embodiments, immunoglobulin Fab fragments may also be displayed on a phage particle (see, e.g., U.S. Pat. No. 5,698,426). Heavy and light chain immunoglobulin cDNA expression libraries may also be prepared in lambda phage, for example, using λImmunoZap™(H) and λImmunoZap™(L) vectors (Stratagene, La Jolla, Calif.). (see Huse et al., supra; see also Sastry et al., supra). Phage display techniques may also be used to select Ig fragments or single chain antibodies that bind to a coronavirus protein. For examples of suitable vectors having multicloning sites into which candidate nucleic acid molecules (e.g., DNA) encoding such antibody fragments or related peptides may be inserted, see, e.g., McLafferty et al., Gene 128:29-36, (1993); Scott et al., Science 249:386-390 (1990); Smith et al., Meth. Enzymol. 217:228-257 (1993); Fisch et al., Proc. Natl. Acad. Sci. USA 93:7761-66 (1996)).

In certain other embodiments, coronavirus protein-specific antibodies are multimeric antibody fragments. Useful methodologies are described generally, for example in Hayden et al., Curr Opin. Immunol. 9:201-12 (1997); Coloma et al., Nat. Biotechnol. 15:159-63 (1997). For example, multimeric antibody fragments may be created by phage techniques to form miniantibodies (U.S. Pat. No. 5,910,573) or diabodies (Holliger et al., Cancer Immunol. Immunother. 45:128-130 (1997)). Multimeric fragments may be generated that are multimers of a coronavirus protein-specific Fv, or that are bispecific antibodies comprising a coronavirus protein-specific Fv noncovalently associated with a second Fv having a different antigen specificity (see, e.g., Koelemij et al., J. Immunother. 22:514-24 (1999)).

Introducing amino acid mutations into coronavirus protein-binding immunoglobulin molecules may be useful to increase the specificity or affinity for a coronavirus protein, or to alter an effector function. Immunoglobulins with higher affinity for the coronavirus protein may be generated by site-directed mutagenesis of particular residues. Computer assisted three-dimensional molecular modeling may be employed to identify the amino acid residues to be changed in order to improve affinity for the coronavirus protein (see, e.g., Mountain et al., Biotechnol. Genet. Eng. Rev. 10:1-142 (1992)). Alternatively, combinatorial libraries of CDRs may be generated in M13 phage and screened for immunoglobulin fragments with improved affinity (see, e.g., Glaser et al., J. Immunol. 149:3903-3913 (1992); Barbas et al., Proc. Natl. Acad. Sci. USA 91:3809-13 (1994); U.S. Pat. No. 5,792,456).

In certain embodiments, the antibody may be genetically engineered to have an altered effector function. For example, the antibody may have an altered capability to mediate complement dependent cytotoxicity (CDC) or antibody dependent cellular cytotoxicity (ADCC). Effector functions may be altered by site-directed mutagenesis (see, e.g., Duncan et al., Nature 332:563-64 (1988); Morgan et al., Immunology 86:319-24 (1995); Eghtedarzedeh-Kondri et al., Biotechniques 23:830-34 (1997)). For example, mutation of the glycosylation site on the Fc portion of the immunoglobulin may alter the capability of the immunoglobulin to fix complement (see, e.g., Wright et al., Trends Biotechnol. 15:26-32 (1997)). Other mutations in the constant region domains may alter the ability of the immunoglobulin to fix complement, or to effect ADCC (see, e.g., Duncan et al., Nature 332:563-64(1988); Morgan et al., Immunology 86:319-24 (1995); Sensel et al., Mol. Immunol. 34:1019-29 (1997)). Alternatively, single chain polypeptides may be constructed recombinantly that comprise an A2E binding fragment, an immunoglobulin hinge region polypeptide, an immunoglobulin CH2 region polypeptide, and an immunoglobulin CH3 region polypeptide (see, e.g., U.S. Patent Publication Nos. 2003/0118592; 2003/0133939).

The nucleic acid molecules encoding an antibody or fragment thereof that specifically binds a coronavirus protein, as described herein, may be propagated and expressed according to any of a variety of well-known procedures for nucleic acid excision, ligation, transformation, and transfection. Thus, in certain embodiments expression of an antibody fragment may be preferred in a prokaryotic host cell, such as Escherichia coli (see, e.g., Pluckthun et al., Methods Enzymol. 178:497-515 (1989)). In certain other embodiments, expression of the antibody or an antigen-binding fragment thereof may be preferred in a eukaryotic host cell, including yeast (e.g., Saccharomyces cerevisiae, Schizosaccharomyces pombe, and Pichia pastoris); animal cells (including mammalian cells); or plant cells. Examples of suitable animal cells include, but are not limited to, myeloma, COS, CHO, or hybridoma cells.

In certain embodiments, anti-idiotype antibodies that recognize an antibody (or antigen-binding fragment thereof) that specifically binds to a coronavirus protein are provided and methods for using these anti-idiotype antibodies. Anti-idiotype antibodies may be generated as polyclonal antibodies or as monoclonal antibodies by the methods described herein, using an anti-coronavirus protein antibody (or antigen-binding fragment thereof) as immunogen. Anti-idiotype antibodies or fragments thereof may also be generated by any of the recombinant genetic engineering methods described above or by phage display selection. An anti-idiotype antibody may react with the antigen-binding site of the anti-coronavirus protein antibody such that binding of the antibody to the coronavirus protein is competitively inhibited. Alternatively, an anti-idiotype antibody as provided herein may not competitively inhibit binding of an anti-coronavirus protein antibody to the coronavirus protein. Anti-idiotype antibodies are useful for immunoassays to determine the presence of anti-coronavirus protein antibodies in a biological sample. For example, an immunoassay such as an ELISA, which are practiced by persons skilled in the art, may be used to determine the presence of an immune response induced by administering (i.e., immunizing) a host with a coronavirus protein as described herein.

All U.S. patents, U.S. patent application publications, U.S. patent applications, foreign patents, foreign patent applications, and non-patent publications referred to in this application, and/or listed in the Application Data Sheet are incorporated herein by reference, in their entireties.

The following examples are offered by way of illustration, and not by way of limitation.

### EXAMPLES

### Example 1

### Preparation of Proteosomes

Immunogens of the instant invention may be combined, admixed, or formulated with proteosomes by way of non-covalent interactions to form a vaccine composition capable of eliciting a protective immune response in an immunized human or animal subject. Proteosomes of the instant application are mucosal adjuvant delivery vehicles comprising outer membrane proteins purified from, for example, Group B type 2 Neisseria meningitidis. The use of proteosomes for the composition (or formulation) of vaccines has been reviewed by Lowell, G. H., in “New Generation Vaccines 2^{nd} ed., Marcel Dekker, Inc., New York, Basil, Hong Kong (1997) pages 193-206. Proteosomes of the instant invention may be prepared by extraction of phenol-killed bacterial paste with a solution of 6% Empigen® BB (EBB) (Albright and Wilson, Whithaven, UK) in 1 M calcium chloride followed by precipitation with ethanol, solubilization in 1% EBB-Tris/EDTA-saline and then precipitation with ammonium sulfate. The precipitates are re-solubilized in the 1% EBB buffer, dialyzed, and stored in 0.1% EBB at −70° C. Alternative processes may be used in the preparation of proteosomes, for example, proteosomes may be prepared by omitting the ammonium sulfate precipitation step to shorten the process. Preparation of proteosomes is disclosed in U.S. Patent Application Publication No. 2001/0053368 and in U.S. Pat. No. 6,476,201 B1.

### Example 2

### Preparation Proteosome: Liposaccharide Immunogenic Composition

A Proteosome adjuvant composition was manufactured by admixing Proteosomes and LPS to allow a presumably non-covalent association. The LPS can be derived from any of a number of gram negative bacteria, such as Shigella, Plesiomonas, Escherichia, or Salmonella species, which is mixed with the Proteosomes prepared as described in Example 1. Briefly, Proteosomes and LPS were thawed overnight at 4° C. and adjusted to 1% Empigen® BB in TEEN buffer. The two components were mixed for 15 minutes at room temperature, at quantities resulting in a final wt/wt ratio of between about 10:1 and about 1:3 of Proteosome:LPS. The Proteosome:LPS mixture was diafiltered on an appropriately sized (e.g., Size 9) 10,000 MWCO hollow fiber cartridge into TNS buffer (0.05 M Tris, 150 mM NaCl pH 8.0). The diafiltration was stopped when Empigen® content in the permeate was <50 ppm (by Empigen® Turbidity Assay or by a Bradford Reagent Assay). The bulk adjuvant (referred to herein as OMP-LPS) was concentrated and adjusted to 5 mg/mL protein (by Lowry assay). Finally, the adjuvant was sterile-filtered using a 0.22 μm Millipak 20 filter unit. The bulk adjuvant was aliquoted into sterile storage containers and frozen.

The OMP-LPS adjuvant was tested for (1) Empigen® (400 ppm) using reverse-phase HPLC; (2) protein content by a Lowry assay; and (3) LPS content by measurement of 2-keto-3-deoxyoctonate (KDO) assay. The OMP-LPS composition was further characterized for particle size distribution as determined by quantitative number weighted analysis using a particle sizer (Brookhaven Instruments model 90 plus or similar machine) (10-100 nm). However, the particle size for the complex may increase or modulate with varying (e.g., higher) Proteosome to LPS ratio. These resultant Proteosome:LPS complexes have been termed Protollin. Current stability data indicate this formulation is stable for over 2 years.

Other versions of Protollin containing modifications of the source of LPS may also be produced as needed. While the nasal adjuvant properties of Protollin were evaluated using Protollin prepared with S. flexneri 2a LPS, Protollin prepared with E. coli LPS has been prepared and found to have similar activity. Advantages to using a Protollin made with E. coli LPS include potentially higher yield of LPS as well as fermentation of bacteria that do not require the containment precautions associated with growing a pathogenic organism, such as S. flexneri. The use of LPS from different sources may also affect induction of protective immunity (adaptive or innate). Accordingly, Protollin was assembled using LPS from two different E. coli in order to compare the level of activity to S. flexneri-based Protollin. These data indicated that E. coli LPS can successfully replace the S. flexneri LPS in Protollin while retaining adjuvant activity. This E. coli Protollin can be compared to the LPS from other well-characterized strains of E. coli, including a strain with LPS that has an O-polysaccharide of sufficient length to solubilize the Proteosome OMP particles during the preparation of Protollin.

Still other versions of Protollin containing modifications of the Proteosome:LPS ratio may also be produced as needed. Initial studies with Protollin were performed with Protollin containing Proteosome OMPs and LPS at a 1:1 weight:weight ratio. However, before advancing efficacy trials in animals or clinical trials in humans with coronavirus antigens it is important to demonstrate the range of OMP:LPS ratios that are active, and investigate the ratio(s) that have optimal adjuvant activity and also retain solubility of the OMP:LPS complexes that constitute Protollin. Accordingly, the same diafiltration technology used previously was used to produce Protollin with several OMP:LPS ratios including ratios of 4:1, 2:1, and 1:1. Ratios ranging from about 4:1 to about 5:1 were included using Protollin composed of both OMP and LPS from Neisseria meningitidis. (Note: N. meningiditis LPS is frequently called LOS denoting lipooligosaccharide to emphasize the fact that the O-side chain of N. meningiditis liposaccharide is shorter than that of other Gram-negative-bacteria such as E. coli and Shigella). Production of Protollin with N. meningiditis LPS (protollin-Nm) is different from all other versions of Protollin. During the production of Proteosome OMPs, N. meningiditis LPS can be removed by ammonium sulfate precipitation techniques so that Proteosome particles have less than 2.5% N. meningiditis LPS. If the LPS is not removed at this step, the resultant Proteosome particles would have 20-25% LPS compared to the amount of Proteosome OMP present, which would be an OMP:LPS ratio ranging from about 5:1 to about 4:1. Thus, Protollin-Nm can be produced in a single step, thereby eliminating further purification of the Proteosome particles as well as the necessity of separately purifying LPS from another organism and then complexing the LPS to Proteosome OMPs. An aliquot of each Protollin was retained for use in, for example, a spin-down assay to verify Proteosome OMP complexing with LPS. Each of these versions of Protollin is tested in mice for adjuvant activity after combining (mixing, admixing, or formulating) with S protein immunogens to make the different versions of Protollin S protein immunogenic compositions (see, e.g., Example 4).

### Example 3

### Preparation and Characterization Recombinant S Protein

In this example, one method for the preparation of native (wild type) Spike protein or fragment thereof is described. Other methods, including synthetic and bacterial expression systems for non-glycosylated S or N protein fragments, are also contemplated. A baculovirus expression system of S. fruiperda Sf9 insect cells (ExpressSF+™) was used. The sequence for the nucleic acid sequence encoding S protein was obtained from Genbank Accession #AY274119 (which represents the entire SARS genome sequence; nucleotides 21493-25259 encode S protein, see FIG. 4). RNA was isolated from a SARS lysate obtained from CDC according to the TRIZOL instruction provided by CDC. This RNA preparation was used to produce cDNA using a TITAN kit (Roche) following the manufacturers instructions. The front end of the S protein encoding nucleic acid sequence was cloned directly into the Baculovirus transfer vector PSC12 using primers 2166 and 2167 (Front: nt 40-750). The middle part (nt 750-2490) and back part (nt 2486-3768) of the S protein encoding nucleic acid sequences were cloned directly into an E. coli pUC 18 vector. Various bacterial clones having correct insets were identified and used to clone the full length S protein encoding nucleic acid sequence into Baculovirus transfer vector PSC12.

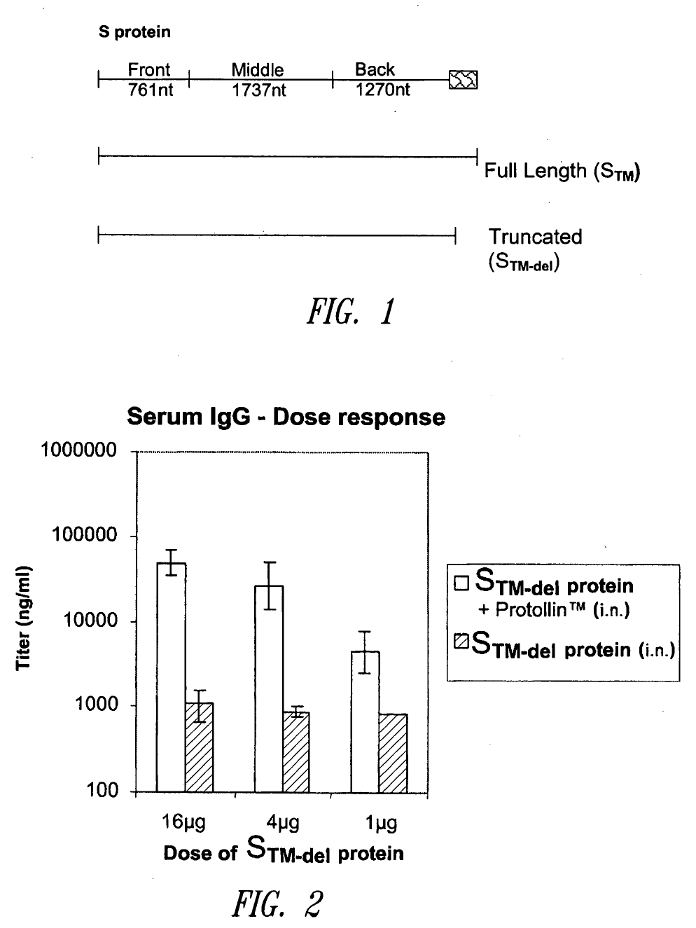

Site-directed mutagenesis was used to create both the S_{TM} full-length construct and the variant S_{TM-del} version (S protein variant lacking the transmembrane domain, see FIG. 1) of the S protein in PSC12. The truncated S_{TM-del} protein is secreted into the media, and then was purified on lentin lectin (LL) and ion-exchange columns resulting in a protein of approximately 75% homogeneity purity. Other purification schemes are also contemplated, such as nickel column purification of histidine epitope tagged S or N proteins fragments or fusion proteins thereof. For example, the full length S_{TM} protein was fused in frame with a His-tag to produce a His-tag fusion protein. Purification of His-tagged proteins was performed by solubilization of a cell pellet with 1% Tergitol, followed by application to and elution from nickel and LL columns. The resulting S_{TM} protein was 95% pure. Both S proteins were bound in Western blot assays by convalescent sera from SARS patients, which shows that recombinantly prepared S protein and variants thereof have native antigenic epitopes specifically bound by S protein antibody.

### Example 4

### Protollin-SARS CoV S-Protein Formulations

Protollin with SARS-CoV S protein or an S protein variant (lacking the transmembrane domain, i.e., S_{TM-del}) were prepared. Mice (10 per group) were immunized intranasally on days 0 and 14, with 16 μg, 4 μg, or 1 μg of purified recombinant S_{TM-del} protein with or without Protollin (1 μg). S_{TM-del} protein (16 μg) was also injected intramuscularly adsorbed onto Alhydrogel® (0.5% w/w) and served as a positive control and for generating serum for establishing ELISA conditions. On day 21, mice were euthanized by asphyxiation with CO₂ and cardiac puncture, and serum and lung lavage fluids harvested and assayed by ELISA for S_{TM-del} specific IgG and IgA levels, respectively. Results are expressed as geometric mean concentrations of antibody.

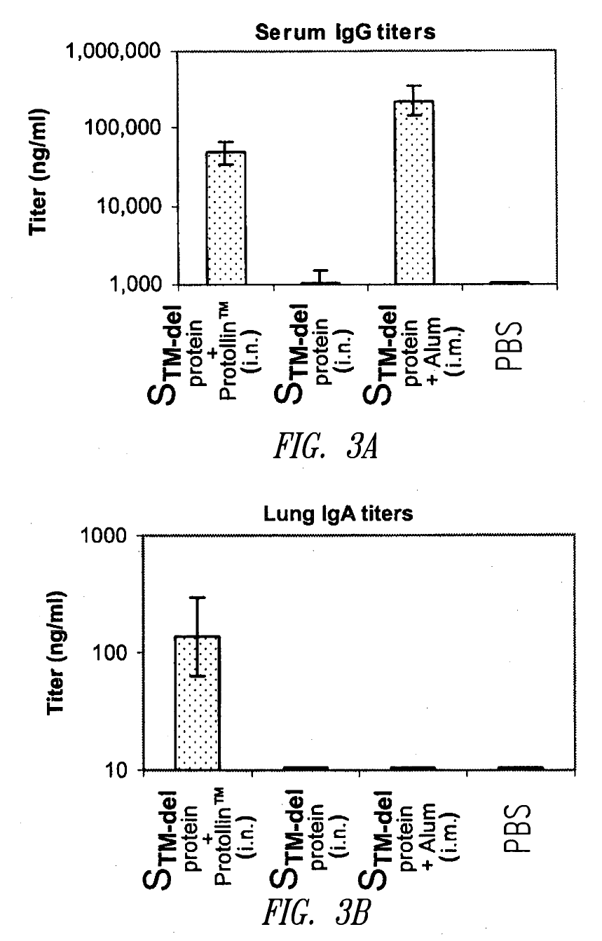

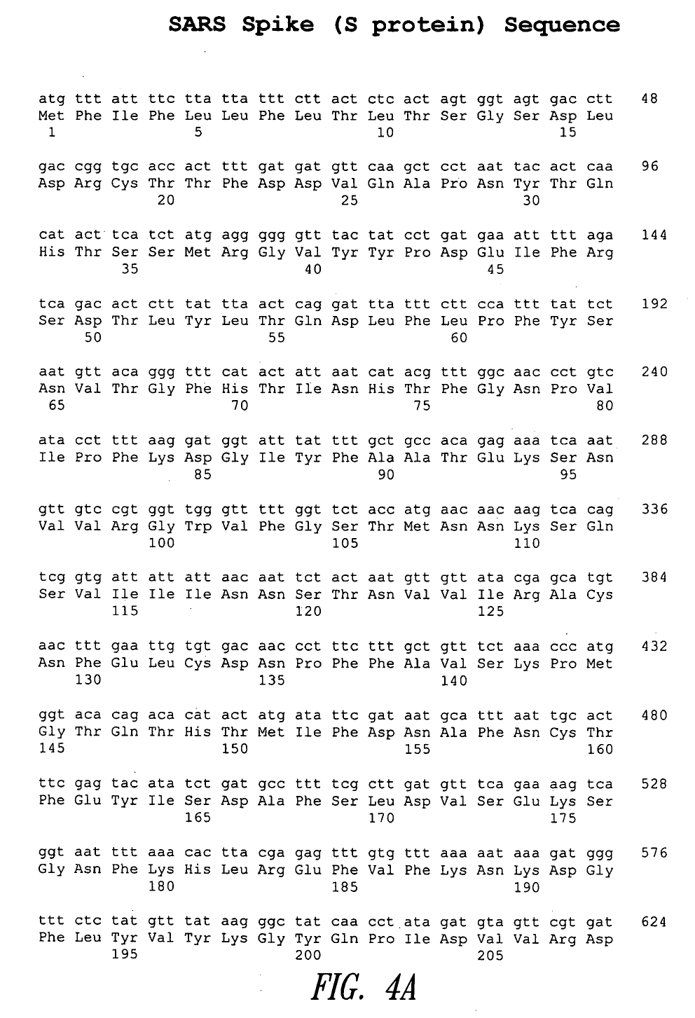

FIG. 2 shows that mice immunized intranasally with Protollin-adjuvanted S_{TM-del} induced up to 48-fold higher levels of antigen-specific serum IgG compared with S_{TM-del} alone given by the same route. The intramuscularly administered, alum adjuvanted S_{TM-del} positive control preparation induced 4-fold higher serum IgG titers compared with those elicited by the intranasal Protollin S_{TM-del} composition (FIG. 3A). However, only the nasal Protollin S_{TM-del} vaccine induced both serum IgG and lung IgA (FIG. 3B).

The data demonstrate that Protollin:S protein variant compositions were capable of inducing antigen-specific serum antibodies that were functionally active (see Example 5) together with a mucosal IgA response. Despite mounting a strong serum IgG and virus neutralizing response, S_{TM-del} protein adjuvanted with alum failed to induce mucosal IgA.

### Example 5

### SARS-CoV Neutralization Assay

This example describes neutralization of infectivity of SARS-CoV by sera from mice immunized with S_{TM-del} protein. In these experiments, aliquots of pre-titered SARS-CoV were mixed with serially diluted samples of individual mouse sera immunized as described in Example 4. Two-fold serial dilutions of mouse sera were prepared beginning with a 1:10 dilution to a final dilution of 1:640; sera were diluted in Minimal Essential Medium (MEM) supplemented with antibiotics, fungizone, amino acids, vitamins, HEPES buffer, and 3% fetal calf serum. To each dilution of sera, SARS-CoV stock virus (100 plaque forming units (pfu) per 50 μl) was diluted to final dilutions of 1:2-1:256. The sera+virus mixtures were gently mixed and then incubated at 37° C. for 2 hours. One hundred microliters of each mixture was transferred to a tissue culture plate (96-well microtiter plate (Corning-Costar)) in which Vero-E6 cells were cultured just to confluency. The cells were incubated with the sera+virus mixtures for 3 days at 37° C. The presence of cytopathic effect (CPE) was determined for each well by microscopy. The neutralizing titer of serum is designated as the serum dilution just lower than the dilution in which CPE was observed.

The serum antibodies induced by the Protollin:S_{TM-del} admixture were functionally active in that they showed neutralization titers of 20, which was four-fold higher than from mice that received the S_{TM-del} antigen alone or PBS. Similarly, serum antibodies induced by the alum-adjuvanted S-protein positive control were able to inhibit replication of virus in vitro resulting in titers of 160.

### Example 6

### Immunogenicity of SARS S-Protein and S_{TM-DEL}

In these experiments, the effect of different doses of Protollin™ on the immunogenicity of a constant dose of full length or S_{TM-del} (ΔTM (transmembrane deleted)) SARS S-protein preparations in anesthetized or non-anesthetized mice. Ten Balb/C mice were included in each group.

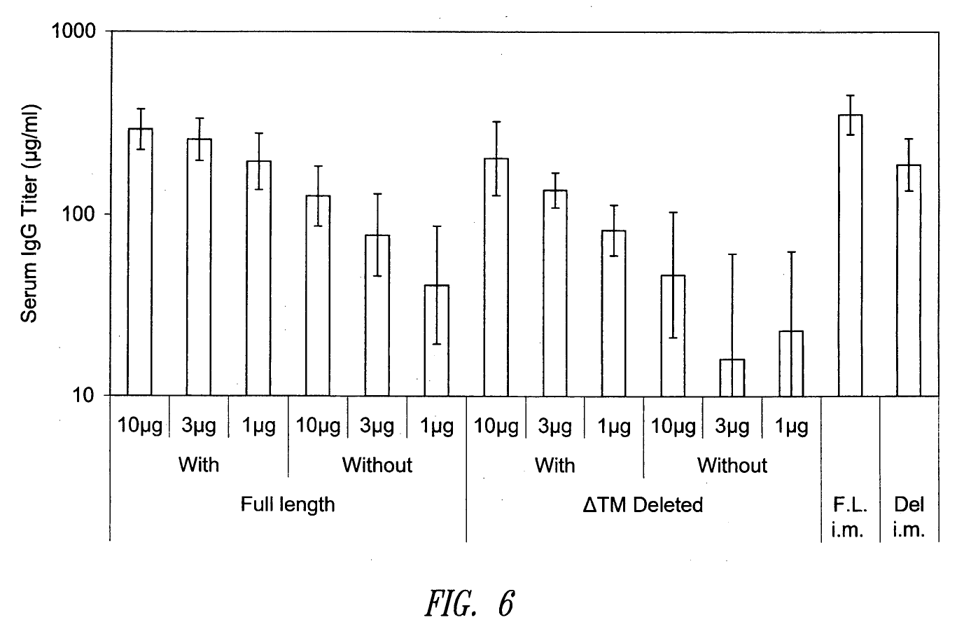

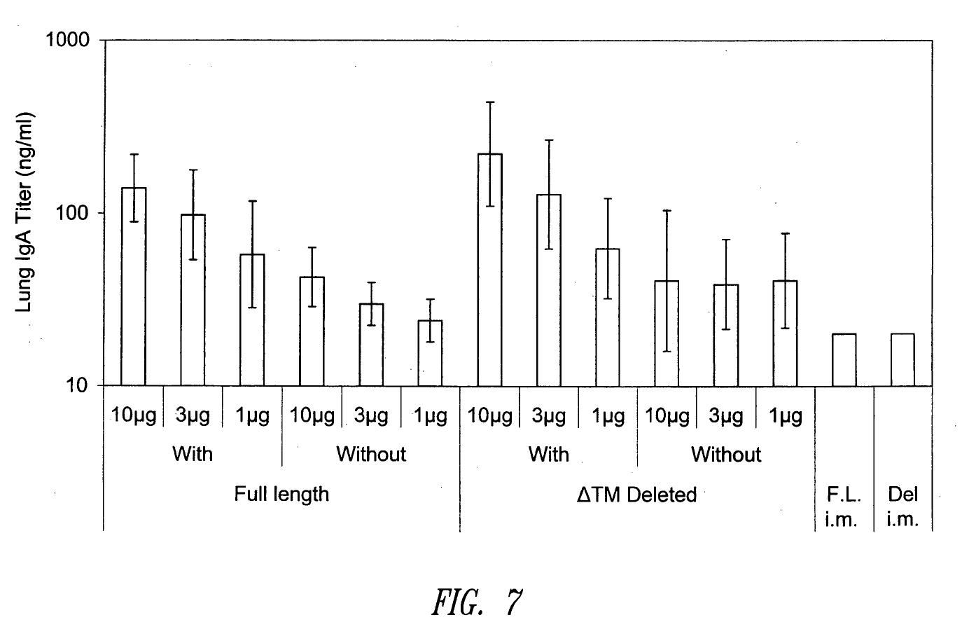

On days 0 and 14, groups of anesthetized or non-anesthetized mice were immunized intranasally with 10 μL containing 10 μg doses of full-length SARS S-protein or S_{TM-del}-protein admixed with 10 μg, 3 μg or 1 μg doses of Protollin™. Additional groups of mice received 25 μL (25 μg of SARS full-length S-protein or 25 μg of SARS S_{TM-del}-protein) adsorbed onto Alhydrogel®(alum), which was administered by intramuscular injection. Control mice received only PBS intranasally. On day 21, mice were euthanized by CO₂ asphyxiation and cardiac puncture. Serum, lung lavage, and nasal wash fluid were harvested. The data are presented in FIG. 6 (serum IgG titer (μg/ml)) and FIG. 7 (lung IgA titer (ng/ml)).

Levels of SARS-specific IgG and IgA antibodies, in individual mice sera and mucosal fluids respectively, were determined by ELISA using plates coated with the appropriate SARS S-protein preparation. Specific IgG and IgA titers were expressed as geometric mean concentrations (ng/ml), the significance of which was assessed by ANOVA analysis (Tukey-Kramer pair-wise comparisons).

Specific serum IgG titers were approximately 2.5 to 5 fold lower in non-anesthetized compared with anesthetized mice. Similarly, mucosal responses were lower in non-anesthetized mice compared with anesthetized mice. Specific IgA was just detectable in non-anesthetized animals, and significant numbers of mice in each non-anesthetized group were non-responders.

At the same dose of Protollin™, serum IgG titers elicited by mice immunized with Protollin™-formulated full length S-protein were approx 1.5-2.5 higher than those elicited by ΔTM-deleted SARS S-protein. The differences were generally not statistically significant except for the ΔTM-deleted SARS S-protein formulated with 1 μg Protollin™, which elicited titers significantly lower than the other vaccines tested (P≦0.001-0.01), and the ΔTM-deleted SARS S-protein admixed with 3 μg of Protollin™, which elicited a serum IgG titer significantly lower than that elicited by the full length S-protein admixed with 10 μg of Protollin™ (P≦0.05). No significant differences were observed between the serum IgG titers elicited by any dose of full length plus Protollin™ admixture (formulation) (or 10 μg ΔTM deleted protein admixed with 10 μg of Protollin™) and either protein adsorbed onto Alhydrogel® and injected intramuscularly.

Specific IgA titers in lung lavage and nasal washes were also determined. Titers significantly above background were observed in all groups of mice given intranasal vaccines but not in any mouse injected with Alhydrogel-adsorbed protein. Dose responses were observed in the groups given intranasal vaccines but none of the differences between the elicited titers was statistically significant.

Neutralization assays were performed as described in Example 5 with sera from mice in this study. The neutralization titers had a highly significant correlation with the IgG titers measured (P≦0.0001).

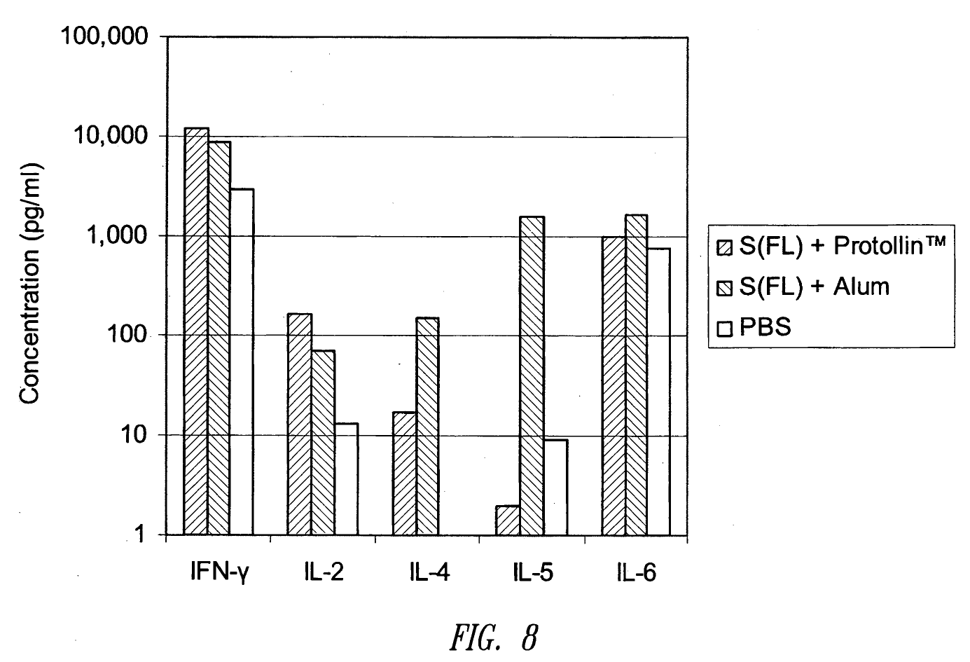

The phenotype of the cellular immune response of the mice in this study was also determined. An assay to determine the phenotype, that is, the cytokine profile, of the response following re-stimulation with full-length S protein was performed with mouse splenocytes. Spleens from mice immunized with full-length S protein (10 μg/ml) and Protollin (10 μg/ml) were pooled and spleens from mice immunized with full-length S protein and alum were pooled, and then both pools were processed into single cell suspensions according to standard methods. The splenic cell suspensions were then incubated with full-length S protein (either 1.7 μg/ml or 5 μg/ml depending upon which cytokine was measured). Cytokines (IFN-γ, IL-2, IL-4, IL-5, and IL-6) released into culture supernatants were determined by quantitative ELISA using OptEIA kits (BD Biosciences, San Jose, Calif.). As shown in FIG. 8, the use of Protollin as an adjuvant skews the immune response toward a type 1 phenotype (including a cellular response) rather than a type 2 phenotype observed in animals immunized with alum.

From the foregoing it will be appreciated that, although specific embodiments of the invention have been described herein for purposes of illustration, various modifications may be made without deviating from the spirit and scope of the invention.

1. A method for treating or preventing a coronavirus infection, comprising administering to a subject in need thereof a composition comprising (a) an adjuvant; (b) a pharmaceutically acceptable excipient; and (c) at least one coronavirus S protein immunogen comprising an amino acid sequence set forth in SEQ ID NO:4, SEQ ID NO:6, SEQ ID NO:8, SEQ ID NO:10, SEQ ID NO:12, SEQ ID NO:14, SEQ ID NO:16, or SEQ ID NO:18, wherein said at least one S protein immunogen is capable of eliciting a protective immune response against coronavirus.

2. The method according to claim 1 wherein the at least one coronavirus S protein immunogen is at least 90% identical to the amino acid sequence set forth in SEQ ID NO:4, SEQ ID NO:6, SEQ ID NO:8, SEQ ID NO:10, SEQ ID NO:12, SEQ ID NO:14, SEQ ID NO:16, or SEQ ID NO:18.

3. The method according to claim 1 wherein the at least one coronavirus S protein immunogen is at least 80% identical to the amino acid sequence set forth in SEQ ID NO:4, SEQ ID NO:6, SEQ ID NO:8, SEQ ID NO:10, SEQ ID NO:12, SEQ ID NO:14, SEQ ID NO: 16, or SEQ ID NO:18.

4. The method according to claim 1 wherein the at least one coronavirus S protein immunogen further comprises a hydrophobic moiety.

5. The method according to claim 4 wherein the hydrophobic moiety is a hydrophobic polypeptide or a lipid.

6. The method according to claim 1 wherein the excipient is a liposome.

7. The method according to claim 1 wherein the adjuvant is a Proteosome or Protollin.

8. The method according to claim 1 wherein the adjuvant is alum, Freund's adjuvant, a Proteosome, or Protollin.

9. The method according to claim 1 wherein the adjuvant is Protollin.

10. The method according to claim 1 wherein at least two S protein immunogens are administered.

11. The method according to claim 1 wherein the at least one coronavirus S protein immunogen is linked to a second amino acid sequence.

12. The method according to claim 11 wherein the at least one coronavirus S protein immunogen is fused to the second amino acid sequence to form a fusion protein.

13. The method according to claim 12 wherein the second amino acid sequence is a tag or an enzyme.

14. The method according to claim 13 wherein the tag is a histidine tag.

15. The method according to claim 1 wherein the coronavirus infection is caused by a group 1 coronavirus, a group 2 coronavirus, a group 3 coronavirus, and a SARS group coronavirus.

16. The method according to claim 1 wherein the coronavirus infection is caused by at least two of a group 1 coronavirus, a group 2 coronavirus, a group 3 coronavirus, and a SARS group coronavirus.

17. The method according to claim 1 wherein the coronavirus infection is caused by a human coronavirus, and wherein the human coronavirus is SARS-CoV.

18. The method according to claim 1 wherein the composition is administered by a route selected from enteral, parenteral, transdermal, transmucosal, nasal, and inhalation.

19. The method according claim 1 wherein the composition is administered nasally.

20. The method according to claim 1 wherein the immune response comprises eliciting at least one antibody that specifically binds to the at least one coronavirus S protein immunogen.

21. A composition comprising (a) at least one coronavirus S protein immunogen that comprises an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26; and (b) a Proteosome or Protollin, wherein said S protein immunogen is capable of eliciting a protective immune response.

22. The composition according to claim 21 the at least one coronavirus S protein immunogen comprises an amino acid sequence at least 90% identical to the amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26.

23. The composition according to claim 21 the at least one coronavirus S protein immunogen comprises an amino acid sequence at least 80% identical to the amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26.

24. The composition according to claim 21 wherein the S protein immunogen further comprises a hydrophobic moiety.

25. The composition according to claim 24 wherein the hydrophobic moiety is a hydrophobic polypeptide or a lipid.

26. The composition according to claim 21 wherein the at least one S protein immunogen is linked to a second amino acid sequence.

27. The composition according to claim 26 wherein the at least one coronavirus S protein immunogen is fused to the second amino acid sequence to form a fusion protein.

28. The composition according to claim 26 wherein the second amino acid sequence is a tag or an enzyme.

29. The composition according to claim 28 wherein the second amino acid sequence is a histidine tag.

30. The composition according to claim 21 wherein the at least one coronavirus S protein immunogen comprises an amino acid sequence set forth in SEQ ID NO: 2.

31. The composition according to 21 wherein the at least one coronavirus S protein immunogen comprises an amino acid sequence set forth in SEQ ID NO: 4.

32. The composition according to claim 21 further comprising a pharmaceutically acceptable excipient.

33. The composition according to claim 21, wherein the at least one S protein immunogen is fused in frame to at least one second S protein immunogen comprising an amino acid sequence selected from an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26 to form a fusion protein.

34. A composition comprising (a) a Proteosome or Protollin; and (b) a multivalent fusion coronavirus immunogen polypeptide.

35. A method for treating or preventing a coronavirus infection, comprising administering to a subject in need thereof the composition according to claim 34.

36. A method for treating or preventing a coronavirus infection, comprising administering to a subject in need thereof a composition comprising: (a) a Proteosome or Protollin; (b) at least one coronavirus S protein immunogen that comprises an amino acid sequence set forth in SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26.

37. The method according to claim 36 wherein the at least one coronavirus S protein immunogen is at least 90% identical to an amino acid sequence set forth in SEQ ID NO:2,SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26.

38. The method according to claim 36 wherein the at least one coronavirus S protein immunogen is at least 80% identical to an amino acid sequence set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or SEQ ID NO:26.

39. The method according to claim 36 wherein the at least one coronavirus S protein immunogen further comprises a hydrophobic moiety.

40. The method according to claim 39 wherein the hydrophobic moiety is a hydrophobic polypeptide or a lipid.

41. The method according to claim 36 wherein the at least one coronavirus S protein immunogen is linked to a second amino acid sequence.

42. The method according to claim 41 wherein the at least one coronavirus S protein irnmunogen is fused to the second amino acid sequence to form a fusion protein.

43. The method according to claim 41 wherein the second amino acid sequence is a tag or an enzyme.

44. The method according to claim 43 wherein the tag is a histidine tag.

45. The method according to claim 36 wherein the coronavirus infection is caused by at least one of a group 1 coronavirus, group 2 coronavirus, a group 3 coronavirus, and a SARS group coronavirus.

46. The method according to claim 36 wherein the coronavirus infection is caused by at least two of a group 1 coronavirus, group 2 coronavirus, group 3 coronavirus, and SARS group coronavirus.

47. The method according to claim 36 wherein the coronavirus infection is caused by a human coronavirus, wherein the human coronavirus is SARS-CoV.

48. The method according to claim 36 wherein the composition is administered by a route selected from enteral, parenteral, transdermal, transmucosal, nasal, and inhalation.

49. The method according to claim 36 wherein the composition is administered nasally.

50. The method according to claim 36 wherein the at least one coronavirus S protein immunogen comprises the amino acid sequence set forth in SEQ ID NO:2.

51. The method according to claim 36 wherein the at least one coronavirus S protein immunogen comprises the amino acid sequence set forth in SEQ ID NO:4.

52. The method according to claim 36 wherein the composition comprises Protollin and at least one coronavirus S protein immunogen, wherein the at least one coronavirus S protein immunogen comprises the amino acid sequence set forth in either SEQ ID NO:2 or SEQ ID NO:4.
